(19) United States
(12) Patent Application Publication
(10) Pub. No.: US 2017/0038387 A1
(43) Pub. Date: Feb. 9, 2017

(54) METHODS FOR TREATING CHRONIC LYMPHOCYTIC LEUKEMIA AND THE USE OF BIOMARKERS AS A PREDICTOR OF CLINICAL SENSITIVITY TO IMMUNOMODULATORY THERAPIES

(71) Applicant: Celgene Corporation, Summit, NJ (US)

(72) Inventors: Anita Gandhi, Bernardsville, NJ (US); Patrick Hagner, Sparta, NJ (US); Anke Klippel, Westfield, NJ (US); Michael Pourdehnad, San Francisco, CA (US); Matthew William Burnell Trotter, Espartinas (ES); Ming Lei, Summit, NJ (US)

(21) Appl. No.: 15/227,877

(22) Filed: Aug. 3, 2016

Related U.S. Application Data
(60) Provisional application No. 62/201,039, filed on Aug. 4, 2015.

(51) Int. Cl. G01N 33/574; A61K 31/454; C12Q 1/68; A61K 31/517

(52) U.S. Cl. CPC G01N 33/57426; A61K 31/517; A61K 31/454; C12Q 1/6886; G01N 33/57484; C12Q 2600/106

(57) ABSTRACT

A method of identifying a subject having chronic lymphocytic leukemia (CLL) who is likely to be responsive to a treatment compound, comprising obtaining a first sample and a second sample from the subject having CLL; administering 3-(5-amino-2-methyl-4-oxo-4H-quinazolin-3-yl)-piperidine-2,6-dione (Compound A) to the first sample and administering lenalidomide to the second sample; determining the level of a biomarker in the first sample and determining the level of the biomarker in the second sample; and diagnosing the subject as being likely to be responsive to the treatment compound if the level of the biomarker in the first sample is different from the level of the biomarker in the second sample.

### 1. FIELD

This application claims the benefit of priority of U.S. provisional application Ser. No. 62/201,039, filed Aug. 4, 2015, the entire contents of which are incorporated herein by reference.

Provided herein are methods for predicting the clinical sensitivity of a cancer, e.g., chronic lymphocytic leukemia (CLL), and a subject's response to treatment with an immunomodulatory agent, such as lenalidomide and 3-(5-amino-2-methyl-4-oxo-4H-quinazolin-3-yl)-piperidine-2,6-dione.

### 2. BACKGROUND

2.1 Cancer and Chronic Lymphocytic Leukemia (CLL)

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (3rd ed., Mosby, St. Louis, Mo., 1993).

There is an enormous variety of cancers which are described in detail in the medical literature. Examples include cancers of the lung, gastric, colon, pancreatic, liver, rectum, prostate, breast, brain, blood and intestine. The incidence of cancer continues to climb as the general population ages, as new cancers develop, and as susceptible populations (e.g., people infected with AIDS or excessively exposed to sunlight) grow. However, options for the treatment of cancer are limited. For example, in the case of blood cancers (e.g., multiple myeloma), few treatment options are available, especially when conventional chemotherapy fails and bone-marrow transplantation is not an option. A tremendous demand therefore exists for new methods and compositions that can be used to treat patients with cancer.

Many types of cancers are associated with new blood vessel formation, a process known as angiogenesis. Several of the mechanisms involved in tumor-induced angiogenesis have been elucidated. The most direct of these mechanisms is the secretion by the tumor cells of cytokines with angiogenic properties. Examples of these cytokines include acidic and basic fibroblastic growth factor (a,b-FGF), angiogenin, vascular endothelial growth factor (VEGF), and TNF-α. Alternatively, tumor cells can release angiogenic peptides through the production of proteases and the subsequent breakdown of the extracellular matrix where some cytokines are stored (e.g., b-FGF). Angiogenesis can also be induced indirectly through the recruitment of inflammatory cells (particularly macrophages) and their subsequent release of angiogenic cytokines (e.g., TNF-α, b-FGF).

Lymphoma refers to cancers that originate in the lymphatic system. Lymphoma is characterized by malignant neoplasms of lymphocytes—B lymphocytes and T lymphocytes (i.e., B-cells and T-cells). Lymphoma generally starts in lymph nodes or collections of lymphatic tissue in organs. Lymphoma may involve the marrow and the blood in some cases. Lymphoma may spread from one site to other parts of the body.

The treatments of various forms of lymphomas are described, for example, in U.S. Pat. No. 7,468,363, the entirety of which is incorporated herein by reference. Such lymphomas include, but are not limited to, Hodgkin's lymphoma, non-Hodgkin's lymphoma, cutaneous B-cell lymphoma, activated B-cell lymphoma, DLBCL, mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma and mantle zone lymphoma and low grade follicular lymphoma.

Leukemia refers to malignant neoplasms of the blood-forming tissues. Various forms of leukemias are described, for example, in U.S. Pat. No. 7,393,862 and U.S. provisional patent application No. 60/380,842, filed May 17, 2002, the entireties of which are incorporated herein by reference. Although viruses reportedly cause several forms of leukemia in animals, causes of leukemia in humans are to a large extent unknown. The Merck Manual, 944-952 (17^{th} ed. 1999). Transformation to malignancy typically occurs in a single cell through two or more steps with subsequent proliferation and clonal expansion. In some leukemias, specific chromosomal translocations have been identified with consistent leukemic cell morphology and special clinical features (e.g., translocations of 9 and 22 in chronic myelocytic leukemia, and of 15 and 17 in acute promyelocytic leukemia). Acute leukemias are predominantly undifferentiated cell populations and chronic leukemias more mature cell forms.

Acute leukemias are divided into lymphoblastic (ALL) and non-lymphoblastic (ANLL) types. The Merck Manual, 946-949 (17^{th} ed. 1999). They may be further subdivided by their morphologic and cytochemical appearance according to the French-American-British (FAB) classification or according to their type and degree of differentiation. The use of specific B- and T-cell and myeloid-antigen monoclonal antibodies are most helpful for classification. ALL is predominantly a childhood disease which is established by laboratory findings and bone marrow examination. ANLL, also known as acute myelogenous leukemia or acute myeloid leukemia (AML), occurs at all ages and is the more common acute leukemia among adults; it is the form usually associated with irradiation as a causative agent.

Chronic leukemias are described as being lymphocytic (CLL) or myelocytic (CIVIL). The Merck Manual, 949-952 (17^{th} ed. 1999).

CLL is a monoclonal disorder characterized by a progressive accumulation of functionally incompetent lymphocytes, and the appearance of mature lymphocytes in blood, bone marrow, and lymphoid organs. The hallmark of CLL is sustained, absolute lymphocytosis (>5,000/μL) and an increase of lymphocytes in the bone marrow. Most CLL patients also have clonal expansion of lymphocytes with B-cell characteristics. CLL is a disease of middle or old age. Symptoms of CLL include enlarged lymph nodes, liver, or spleen, recurring infections, loss of appetite or early satiety, abnormal bruising (late-stage symptom), fatigue, night sweats, and so on.

Patients with CLL have a higher-than-normal white blood cell count, which is determined by complete blood count (CBC). Peripheral blood flow cytometry is one of the most valuable tests to confirm a diagnosis of CLL. Other tests that may be helpful for diagnosis include bone marrow biopsy and ultrasonography of the liver and spleen. Immunoglobulin testing may be indicated for patients developing repeated infections.

In CIVIL, the characteristic feature is the predominance of granulocytic cells of all stages of differentiation in blood, bone marrow, liver, spleen, and other organs. In the symptomatic patient at diagnosis, the total white blood cell (WBC) count is usually about 200,000/μL, but may reach 1,000,000/μL. CIVIL is relatively easy to diagnose because of the presence of the Philadelphia chromosome.

Bone marrow stromal cells are well known to support CLL disease progression and resistance to chemotherapy. Disrupting the interactions between CLL cells and stromal cells is an additional target of CLL chemotherapy.

In addition to the acute and chronic categorization, neoplasms are also categorized based upon the cells giving rise to such disorder into precursor or peripheral. See e.g., U.S. patent Publication No. 2008/0051379, the disclosure of which is incorporated herein by reference in its entirety. Precursor neoplasms include ALLs and lymphoblastic lymphomas and occur in lymphocytes before they have differentiated into either a T- or B-cell. Peripheral neoplasms are those that occur in lymphocytes that have differentiated into either T- or B-cells. Such peripheral neoplasms include, but are not limited to, B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue, nodal marginal zone lymphoma, splenic marginal zone lymphoma, hairy cell leukemia, plasmacytoma, DLBCL and Burkitt lymphoma. In over 95 percent of CLL cases, the clonal expansion is of a B cell lineage. See Cancer: Principles & Practice of Oncology (3rd Edition) (1989) (pp. 1843-1847). In less than 5 percent of CLL cases, the tumor cells have a T-cell phenotype. Notwithstanding these classifications, however, the pathological impairment of normal hematopoiesis is the hallmark of all leukemias.

There exists a significant need for safe and effective methods of treating, preventing and managing cancer, e.g., CLL, while reducing or avoiding the toxicities and/or side effects associated with the conventional therapies. The present invention satisfies these and other needs.

2.2 Compounds

A number of studies have been conducted with the aim of providing compounds that can safely and effectively be used to treat diseases associated with abnormal production of TNF-α. See, e.g., Marriott, J. B., et al., Expert Opin. Biol. Ther., 2001, 1(4): 1-8; G. W. Muller, et al., J Med Chem., 1996, 39(17): 3238-3240; and G. W. Muller, et al., Bioorg & Med Chem Lett., 1998, 8: 2669-2674. Some studies have focused on a group of compounds selected for their capacity to potently inhibit TNF-α production by LPS stimulated PBMC. L. G. Corral, et al., Ann. Rheum. Dis., 1999, 58:(Suppl I) 1107-1113. These compounds show not only potent inhibition of TNF-α but also marked inhibition of LPS induced monocyte IL1β and IL12 production. LPS induced IL6 is also inhibited by such compounds, albeit partially. These compounds are potent stimulators of LPS induced IL10. Id.

Thalidomide, lenalidomide and pomalidomide have shown remarkable responses in patients with multiple myeloma, lymphoma and other hematological diseases such as myelodysplastic syndrome. See Galustian C, et al., Expert Opin Pharmacother., 2009, 10:125-133. These treatment compounds display a broad spectrum of activity, including anti-angiogenic properties, modulation of pro-inflammatory cytokines, co-stimulation of T cells, increased NK cell toxicity, direct anti-tumor effects and modulation of stem cell differentiation.

For example, thalidomide and lenalidomide have emerged as important options for the treatment of multiple myeloma in newly diagnosed patients, in patients with advanced disease who have failed chemotherapy or transplantation, and in patients with relapsed or refractory multiple myeloma. Lenalidomide in combination with dexamethasone has been approved for the treatment of patients with multiple myeloma who have received at least one prior therapy. Pomalidomide may also be administered in combination with dexamethasone. U.S. Patent Publication No. 2004/0029832 A1, the disclosure of which is hereby incorporated in its entirety, discloses the treatment of multiple myeloma.

Another compound provided herein is 3-(5-amino-2-methyl-4-oxo-4H-quinazolin-3-yl)-piperidine-2,6-dione (“Compound A”), or an enantiomer or a mixture of enantiomers thereof; or a pharmaceutically acceptable salt, solvate, hydrate, co-crystal, clathrate, or polymorph thereof. Compound A can be prepared as described in U.S. Pat. No. 7,635,700, the disclosure of which is incorporated herein by reference in its entirety. The compound can be also synthesized according to other methods apparent to those of skill in the art based upon the teaching herein. In certain embodiments, Compound A is in a crystalline form described in U.S. Provisional Pat. App. No. 61/451,806, filed Mar. 11, 2011, which is incorporated herein by reference in its entirety. In some embodiments, the hydrochloride salt of Compound A is used in the methods provided herein. Methods of treating, preventing and/or managing cancers and other diseases using Compound A are described in U.S. Provisional Pat. App. No. 61/451,995, filed Mar. 11, 2011, which is incorporated herein by reference in its entirety.

The conventional methods of assessing the effects of immunomodulatory compounds require live cellular assays or lengthy clinical endpoints. These cellular tests are cumbersome and often require the use of various stimulants (e.g., lipopolysaccharide or anti-CD3 antibody). Indirect endpoints such as cytokine production are evaluated, which can be influenced via multiple pathways. Further, clinical efficacy of these compounds could not be correctly predicted, as it could only be measured in terms of patient response, which usually requires a minimum of several months of treatment. In view of the deficiencies of the conventional methods, there is a need to develop an efficient, sensitive and accurate method to detect, quantify and characterize the pharmacodynamic activity of immunomodulatory compounds.

### 3. SUMMARY OF THE INVENTION

In one aspect, provided herein is a method of identifying a subject having chronic lymphocytic leukemia (CLL) who is likely to be responsive to a treatment compound, comprising:

(a) obtaining a first sample and a second sample from the subject having CLL;

(b) administering 3-(5-amino-2-methyl-4-oxo-4H-quinazolin-3-yl)-piperidine-2,6-dione (Compound A) to the first sample and administering lenalidomide to the second sample;

(c) determining the level of a biomarker in the first sample and determining the level of the biomarker in the second sample, wherein the biomarker is selected from the group consisting of biomarkers identified in Tables 1-3; and

(d) diagnosing the subject as being likely to be responsive to the treatment compound if the level of the biomarker in the first sample is different from the level of the biomarker in the second sample,

wherein the treatment compound is Compound A or lenalidomide.

In another aspect, provided herein is a method of predicting the responsiveness of a subject having or suspected of having CLL to a treatment compound, comprising:

(a) obtaining a first sample and a second sample from the subject;

(b) administering Compound A to the first sample and administering lenalidomide to the second sample;

(c) determining the level of a biomarker in the first sample and determining the level of the biomarker in the second sample, wherein the biomarker is selected from the group consisting of biomarkers identified in Tables 1-3; and

(d) diagnosing the subject as being likely to be responsive to the treatment compound if the level of the biomarker in the first sample is different from the level of the biomarker in the second sample,

wherein the treatment compound is Compound A or lenalidomide.

In another aspect, provided herein is a method of treating CLL in a subject, comprising:

(a) obtaining a first sample and a second sample from the subject;

(b) administering Compound A to the first sample and administering lenalidomide to the second sample;

(c) determining the level of a biomarker in the first sample and determining the level of the biomarker in the second sample, wherein the biomarker is selected from the group consisting of biomarkers identified in Tables 1-3;

(d) diagnosing the subject as being likely to be responsive to a treatment compound if the level of the biomarker in the first sample is different from the level of the biomarker in the second sample; and

(e) administering a therapeutically effective amount of the treatment compound to the subject diagnosed to be likely to be responsive to the treatment compound,

wherein the treatment compound is Compound A or lenalidomide.

In some embodiments, the biomarker is selected from the group consisting of biomarkers identified in Table 1, and diagnosing the subject as being likely to be responsive to both Compound A and lenalidomide if the level of the biomarker in the first sample is different from the level of the biomarker in the second sample.

In some embodiments, the biomarker is selected from the group consisting of BCL2L1, GZMB, IGHM, IRF8, KLF13, LGALS9, MARCKS, NDE1, NFKBIE, SNX20, TCF7, WIZ, and ZBTB10.

In some embodiments, the biomarker is selected from the group consisting of BCL2L1, MARCKS, NDE1, and ZBTB10, and diagnosing the subject as being likely to be responsive to both Compound A and lenalidomide if the level of the biomarker in the first sample is higher than the level of the biomarker in the second sample.

In some embodiments, the biomarker is selected from the group consisting of GZMB, IGHM, IRF8, KLF13, LGALS9, NFKBIE, SNX20, TCF7, and WIZ, and diagnosing the subject as being likely to be responsive to both Compound A and lenalidomide if the level of the biomarker in the first sample is lower than the level of the biomarker in the second sample.

In a specific embodiment, the biomarker is BCL2L1. In another specific embodiment, the biomarker is GZMB. In another specific embodiment, the biomarker is IGHM. In yet another embodiment, the biomarker is IRF8. In yet another embodiment, the biomarker is KLF13. In yet another embodiment, the biomarker is LGALS9. In yet another embodiment, the biomarker is MARCKS. In yet another embodiment, the biomarker is NDE1. In yet another embodiment, the biomarker is NFKBIE. In yet another embodiment, the biomarker is SNX20. In yet another embodiment, the biomarker is TCF7. In yet another embodiment, the biomarker is WIZ. In yet another embodiment, the biomarker is ZBTB10.

In some embodiments, the method provided herein comprises administering a therapeutically effective amount of Compound A or lenalidomide to the subject diagnosed to be likely to be responsive to both Compound A and lenalidomide.

In some embodiments, the biomarker is selected from the group consisting of biomarkers identified in Tables 2-3, and diagnosing the subject as being likely to be more responsive to Compound A than to lenalidomide if the level of the biomarker in the first sample is different from the level of the biomarker in the second sample.

In some embodiments, the biomarker is selected from the group consisting of IKZF3, CD40, CR2, CTSS, GBP1, ISG20, SASH1, and SEMA7A.

In some embodiments, the biomarker is selected from the group consisting of CD40, CR2, CTSS, GBP1, ISG20, SASH1, and SEMA7A, and diagnosing the subject as being likely to be more responsive to Compound A than to lenalidomide if the level of the biomarker in the first sample is higher than the level of the biomarker in the second sample.

In other embodiments, the biomarker is IKZF3, and diagnosing the subject as being likely to be more responsive to Compound A than to lenalidomide if the level of the biomarker in the first sample is lower than the level of the biomarker in the second sample.

In a specific embodiment, the biomarker is CD40. In another specific embodiment, the biomarker is CR2. In another specific embodiment, the biomarker is CTSS. In yet another embodiment, the biomarker is GBP1. In yet another embodiment, the biomarker is ISG20. In yet another embodiment, the biomarker is SASH1. In yet another embodiment, the biomarker is SEMA7A.

In some embodiments, the method provided herein comprises administering a therapeutically effective amount of Compound A to the subject diagnosed to be likely to be more responsive to Compound A than to lenalidomide.

In some embodiments, the biomarker is selected from the group consisting of biomarkers identified in Tables 1-3, diagnosing the subject as being likely to be responsive to Compound A if the level of the biomarker in the first sample is different from the level of the biomarker in the second sample.

In some embodiments, the biomarker is selected from the group consisting of BCL2L1, GZMB, IGHM, IKZF1, IRF8, KLF13, LGALS9, MARCKS, NDE1, NFKBIE, PTK2B, SAMSN1, SELL, SLAMF1, SNX20, SOD2, TCF7, TRAF1, WIZ, ZBTB10, IKZF3, CD40, CR2, CTSS, GBP1, ISG20, SASH1, and SEMA7A.

In some embodiments, the biomarker is selected from the group consisting of PTK2B, SAMSN1, SLAMF1, SOD2, and TRAF1, and diagnosing the subject as being likely to be responsive to Compound A if the level of the biomarker in the first sample is higher than the level of the biomarker in the second sample.

In other embodiments, the biomarker is selected from the group consisting of IKZF1 and SELL, and diagnosing the subject as being likely to be responsive to Compound A if the level of the biomarker in the first sample is lower than the level of the biomarker in the second sample.

In a specific embodiment, the biomarker is IKZF1. In another specific embodiment, the biomarker is PTK2B. In another specific embodiment, the biomarker is SAMSN1. In yet another embodiment, the biomarker is SELL. In yet another embodiment, the biomarker is SLAMF1. In yet another embodiment, the biomarker is SOD2. In yet another embodiment, the biomarker is TRAF1.

In some embodiments, the method provided herein comprises administering a therapeutically effective amount of Compound A to the subject diagnosed to be likely to be responsive to Compound A.

In some embodiments, the level of the biomarker is determined by comparing to a reference level of the biomarker of a control sample, and wherein the control sample is obtained from the subject prior to administering Compound A or lenalidomide; and wherein the control sample is from the same source as the first and the second samples.

In other embodiments, the level of the biomarker is determined by comparing to a reference level of the biomarker of a control sample, and wherein the control sample is obtained from a healthy subject not having CLL; and wherein the control sample is from the same source as the first and the second samples.

In another aspect, provided herein is a method of identifying a subject having CLL who is not likely to be responsive to a treatment compound or predicting the responsiveness of a subject having or suspected of having CLL to a treatment compound, comprising:

(a) obtaining a first sample and a second sample from the subject;

(b) administering Compound A to the first sample and administering lenalidomide to the second sample;

(c) determining the level of a biomarker in the first sample and determining the level of the biomarker in the second sample, wherein the biomarker is PDE6D; and

(d) diagnosing the subject as not being likely to be responsive to the treatment compound if the level of the biomarker in the first sample is different from the level of the biomarker in the second sample,

wherein the treatment compound is Compound A or lenalidomide.

In yet another aspect, provided herein is a method of identifying a subject having CLL who is likely to be responsive to Compound A, comprising:

(a) obtaining a sample from the subject having CLL;

(b) administering Compound A to the sample;

(c) determining the level of a biomarker in the sample, wherein the biomarker is selected from the group consisting of biomarkers identified in Table 5; and

(d) diagnosing the subject as being likely to be responsive to Compound A if the level of the biomarker in the sample is different from a reference level of the biomarker in a control sample, wherein the control sample is obtained from a subject not responsive to Compound A.

In yet another aspect, provided herein is a method of predicting the responsiveness of a subject having or suspected of having CLL to Compound A, comprising:

(a) obtaining a sample from the subject;

(b) administering Compound A to the sample;

(c) determining the level of a biomarker in the sample, wherein the biomarker is selected from the group consisting of biomarkers identified in Table 5; and

(d) diagnosing the subject as being likely to be responsive to Compound A if the level of the biomarker in the sample is different from a reference level of the biomarker in a control sample, wherein the control sample is obtained from a subject not responsive to Compound A.

In yet another aspect, provided herein is a method of treating CLL in a subject, comprising:

(a) obtaining a sample from the subject having CLL;

(b) administering Compound A to the sample;

(c) determining the level of a biomarker in the sample, wherein the biomarker is selected from the group consisting of biomarkers identified in Table 5; and

(d) diagnosing the subject as being likely to be responsive to Compound A if the level of the biomarker in the sample is different from a reference level of the biomarker in a control sample, wherein the control sample is obtained from a subject not responsive to Compound A; and

(e) administering a therapeutically effective amount of Compound A to the subject diagnosed to be likely to be responsive to Compound A.

In some embodiments, the biomarker is selected from the group consisting of APOBEC3G, APOC3, APOL2, CR2, CTSS, FCHSD2, GBP2, GBP4, ICAM1, IDI1, IGHM, IKZF1, IKZF3, IL4I1, IRF5, IRF8, ISG20, KYNU, LAP3, LGALS9, NCF2, NCF4, NDE1, NECAP2, OAS1, PARP14, PDE6D, PLEK, PNP, PPA1, PPP1R18, RELB, SAMSN1, SEMA7A, SLFN5, SOD2, TAPBP, TNIP1, TRAF1, TRIP10, and ZFP91.

In a specific embodiment, the biomarker is GBP4. In another specific embodiment, the biomarker is LGALS9. In another specific embodiment, the biomarker is IRF5.

In one aspect, provided herein is a method of identifying a subject having CLL who is likely to be responsive to a treatment compound, comprising:

(a) obtaining a sample from the subject;

(b) administering the treatment compound to the sample;

(c) determining the level of a biomarker in the sample, wherein the biomarker is PDE6D; and

(d) diagnosing the subject as being likely to be responsive to the treatment compound if the level of the biomarker in the sample is different from a reference level of the biomarker in a control sample, wherein the control sample is obtained from a subject not responsive to the treatment compound;

wherein the treatment compound is Compound A or lenalidomide.

In certain embodiments, the subject is identified as being likely to be responsive to the treatment compound if the level of the biomarker PDE6D in the sample is lower than the level of the biomarker in the control sample.

In another aspect, provided herein is a method of predicting the responsiveness of a subject having or suspected of having CLL to a treatment compound, comprising:

(a) obtaining a sample from the subject;

(b) administering the treatment compound to the sample;

(c) determining the level of a biomarker in the sample, wherein the biomarker is PDE6D; and

(d) diagnosing the subject as being likely to be responsive to the treatment compound if the level of the biomarker in the sample is different from a reference level of the biomarker in a control sample, wherein the control sample is obtained from a subject not responsive to the treatment compound;

wherein the treatment compound is Compound A or lenalidomide.

In certain embodiments, the subject is diagnosed as being likely to be responsive to the treatment compound if the level of the biomarker PDE6D in the sample is lower than the level of the biomarker in the control sample.

In yet another aspect, provided herein is a method of treating CLL in a subject, comprising:

(a) obtaining a sample from the subject;

(b) administering the treatment compound to the sample;

(c) determining the level of a biomarker in the sample, wherein the biomarker is PDE6D;

(d) diagnosing the subject as being likely to be responsive to the treatment compound if the level of the biomarker in the sample is different from a reference level of the biomarker in a control sample, wherein the control sample is obtained from a subject not responsive to the treatment compound; and

(e) administering a therapeutically effective amount of the treatment compound to the subject diagnosed to be likely to be responsive to the treatment compound;

wherein the treatment compound is Compound A or lenalidomide.

In certain embodiments, the subject is diagnosed as being likely to be responsive to the treatment compound if the level of the biomarker PDE6D in the sample is lower than the level of the biomarker in the control sample, and the treatment compound is administered.

In some embodiments, the level of the biomarker is measured by determining the protein level of the biomarker.

In some embodiments, the method provided herein comprises contacting proteins within the sample with a first antibody that immunospecifically binds to the biomarker protein.

In some embodiments, the method provided herein further comprises (i) contacting the biomarker protein bound to the first antibody with a second antibody with a detectable label, wherein the second antibody immunospecifically binds to the biomarker protein, and wherein the second antibody immunospecifically binds to a different epitope on the biomarker protein than the first antibody; (ii) detecting the presence of the second antibody bound to the proteins; and (iii) determining the amount of the biomarker protein based on the amount of the detectable label in the second antibody.

In other embodiments, the method provided herein further comprises (i) contacting the biomarker protein bound to the first antibody with a second antibody with a detectable label, wherein the second antibody immunospecifically binds to the first antibody; (ii) detecting the presence of the second antibody bound to the proteins; and (iii) determining the amount of the biomarker protein based on the amount of the detectable label in the second antibody.

In other embodiments, the level of the biomarker is measured by determining the mRNA level of the biomarker.

In other embodiments, the level of the biomarker is measured by determining the cDNA level of the biomarker.

In some embodiments, the level of the biomarker is measured using quantitative PCR (qPCR).

### 4. BRIEF DESCRIPTION OF THE FIGURES

FIGS. 1A-1D show the effects of Compound A or lenalidomide on samples from four groups of CLL patients: Case B1 (FIG. 1A), Case B2 (FIG. 1B), Case B3 (FIG. 1C), and Case B4 (FIG. 1D).

FIGS. 2A-2D show distribution of normalized relative abundances for each replicate of the four case scenarios: Case B1 (FIG. 2A), Case B2 (FIG. 2B), Case B3 (FIG. 2C), and Case B4 (FIG. 2D).

FIGS. 3A-3D are heatmaps representing correlations of log 2 fold-changes of protein levels post compound treatment as compared with DMSO (control) across conditions for the four case scenarios: Case B1 (FIG. 3A), Case B2 (FIG. 3B), Case B3 (FIG. 3C), and Case B4 (FIG. 3D).

FIGS. 4A-4D show log 2 ratios of relative proteins abundances for lenalidomide and Compound A exposed samples (10 μM, 24 hours) against corresponding DMSO controls (averaged across replicates) for each case scenario: Case B1 (FIG. 4A), Case B2 (FIG. 4B), Case B3 (FIG. 4C), and Case B4 (FIG. 4D).

FIG. 5 is a heatmap including proteins differentially affected by lenalidomide versus Compound A (10 μM, 24 hours).

FIGS. 6A-6C represent log 2 fold-changes of relative protein abundances upon 24 hour exposure to 10 μM Compound A versus DMSO control for three case scenarios: Case B1 (FIG. 6A), Case B2 (FIG. 6B), and Case B3 (FIG. 6C).

FIG. 7 shows proteins identified as differentially perturbed in Case B1, B2 and B3 as compared with Case B4.

FIGS. 8A-8D show −log 10 (FOR q-value) of pathways significantly associated with increased protein abundance levels upon treatment with lenalidomide or Compound A (10 μM, 24 hours) for each case scenario: Case B1 (FIG. 8A), Case B2 (FIG. 8B), Case B3 (FIG. 8C), and Case B4 (FIG. 8D).

FIGS. 9A-9C show −log 10 (FDR q-value) of pathways significantly associated with decreased protein abundance levels upon treatment with lenalidomide or Compound A (10 μM, 24 hours) for three case scenarios: Case B1 (FIG. 9A), Case B3 (FIG. 9B), and Case B4 (FIG. 9C).

FIGS. 10A-10B show correlation of PDE6D downregulation with growth inhibition. FIG. 10A is a scatter plot of normalized cell growth against normalized residual PDE6D protein levels. FIG. 10B shows linear regression for the correlation between normalized cell growth and normalized residual PDE6D protein levels.

### 5. DETAILED DESCRIPTION OF THE INVENTION

The methods provided herein are based, in part, on the finding that the levels of certain proteins change differently in response to lenalidomide treatment as compared with Compound A treatment, and that the set of differentially affected proteins by lenalidomide versus Compound A is different in different patient groups. Thus, such differentially affected proteins may be used to associate a patient to a specific patient group or as biomarkers for selecting a patient belonging to a specific patient group.

The methods provided herein are also based, in part, on the finding that the levels of certain proteins change differently in response to Compound A treatment in different patient groups. Thus, these proteins can also be used to select patients who are responsive to Compound A treatment.

### 5.1 Definitions

As used herein, and unless otherwise specified, the terms “treat,” “treating” and “treatment” refer to an action that occurs while a patient is suffering from the specified cancer, which reduces the severity of the cancer, or retards or slows the progression of the cancer.

The term “sensitivity” and “sensitive” when made in reference to treatment with compound is a relative term which refers to the degree of effectiveness of the compound in lessening or decreasing the progress of a tumor or the disease being treated. For example, the term “increased sensitivity” when used in reference to treatment of a cell or tumor in connection with a compound refers to an increase of, at least a 5%, or more, in the effectiveness of the tumor treatment.

As used herein, the terms “compound” and “treatment compound” are used interchangeably, and include immunomodulatory compound or immunomodulatory drug. As used herein, the term “immunomodulatory compound” or “immunomodulatory drug” refers generally to a molecule or agent capable of altering the immune response in some way.

As used herein, and unless otherwise specified, the term “therapeutically effective amount” of a compound is an amount sufficient to provide a therapeutic benefit in the treatment or management of a cancer, or to delay or minimize one or more symptoms associated with the presence of the cancer. A therapeutically effective amount of a compound means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent. The term also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “responsiveness” or “responsive” when used in reference to a treatment refer to the degree of effectiveness of the treatment in lessening or decreasing the symptoms of a disease, e.g., CLL, being treated. For example, the term “increased responsiveness” when used in reference to a treatment of a cell or a subject refers to an increase in the effectiveness in lessening or decreasing the symptoms of the disease when measured using any methods known in the art. In certain embodiments, the increase in the effectiveness is at least about 5%, at least about 10%, at least about 20%, at least about 30%, at least about 40%, or at least about 50%.

As used herein, the terms “effective subject response,” “effective patient response,” or “effective patient tumor response” refers to any increase in the therapeutic benefit to the patient. An “effective patient tumor response” can be, for example, a 5%, 10%, 25%, 50%, or 100% decrease in the rate of progress of the tumor. An “effective patient tumor response” can be, for example, a 5%, 10%, 25%, 50%, or 100% decrease in the physical symptoms of a cancer. An “effective patient tumor response” can also be, for example, a 5%, 10%, 25%, 50%, 100%, 200%, or more increase in the response of the patient, as measured by any suitable means, such as gene expression, cell counts, assay results, tumor size, etc.

An improvement in the cancer or cancer-related disease can be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

The term “likelihood” or “likely” generally refers to an increase in the probability of an event. The term “likelihood” or “likely” when used in reference to the effectiveness of a patient tumor response generally contemplates an increased probability that the rate of tumor progress or tumor cell growth will decrease. The term “likelihood” or “likely” when used in reference to the effectiveness of a patient tumor response can also generally mean the increase of indicators, such as mRNA or protein expression, that may evidence an increase in the progress in treating the tumor.

The term “predict” generally means to determine or tell in advance. When used to “predict” the effectiveness of a cancer treatment, for example, the term “predict” can mean that the likelihood of the outcome of the cancer treatment can be determined at the outset, before the treatment has begun, or before the treatment period has progressed substantially.

The term “monitor,” as used herein, generally refers to the overseeing, supervision, regulation, watching, tracking, or surveillance of an activity. For example, the term “monitoring the effectiveness of a compound” refers to tracking the effectiveness in treating a cancer in a patient or in a tumor cell culture. Similarly, the “monitoring,” when used in connection with patient compliance, either individually, or in a clinical trial, refers to the tracking or confirming that the patient is actually taking a drug being tested as prescribed. The monitoring can be performed, for example, by following the expression of mRNA or protein biomarkers.

“Tumor,” as used herein, refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

The terms “cancer” and “cancerous” refer to or describe the physiological condition in mammals that is typically characterized by unregulated cell growth. Examples of cancer include, but are not limited to, blood-borne tumors (e.g., multiple myeloma, lymphoma and leukemia), and solid tumors.

The term “regulate” as used herein refers to controlling the activity of a molecule or biological function, such as enhancing or diminishing the activity or function.

The term “refractory or resistant” refers to a circumstance where patients, even after intensive treatment, have residual cancer cells (e.g., lymphoma cells) in their lymphatic system, blood and/or blood forming tissues (e.g., marrow).

As used herein the terms “polypeptide” and “protein” as used interchangeably herein, refer to a polymer of amino acids of three or more amino acids in a serial array, linked through peptide bonds. The term “polypeptide” includes proteins, protein fragments, protein analogues, oligopeptides and the like. The term polypeptide as used herein can also refer to a peptide. The amino acids making up the polypeptide may be naturally derived, or may be synthetic. The polypeptide can be purified from a biological sample. The polypeptide, protein, or peptide also encompasses modified polypeptides, proteins, and peptides, e.g., a glycopolypeptide, glycoprotein, or glycopeptide; or a lipopolypeptide, lipoprotein, or lipopeptide.

The term “antibody” is used herein in the broadest sense and covers fully assembled antibodies, antibody fragments which retain the ability to specifically bind to the antigen (e.g., Fab, F(ab′)₂, Fv, and other fragments), single chain antibodies, diabodies, antibody chimeras, hybrid antibodies, bispecific antibodies, humanized antibodies, and the like. The term “antibody” covers both polyclonal and monoclonal antibodies. The term “antibody” and “immunoglobulin” or “Ig” may be used interchangeably herein.

Antibodies provided herein include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), camelized antibodies, Fab fragments, F(ab″) fragments, disulfide-linked Fvs (sdFv), anti-idiotypic (anti-Id) antibodies, and epitope-binding fragments of any of the above. In particular, antibodies provided herein include immunoglobulin molecules and immunologically active portions of immunoglobulin molecules, i.e., antigen binding domains or molecules that contain an antigen-binding site that immunospecifically binds to a biomarker provided herein. The antibodies provided herein can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule. In certain embodiments, antibodies provided herein are IgG antibodies, or a class (e.g., human IgG1 or IgG4) or subclass thereof.

The term “antigen binding domain,” “antigen binding region,” “antigen binding fragment,” and similar terms refer to that portion of an antibody which comprises the amino acid residues that interact with an antigen and confer on the binding agent its specificity and affinity for the antigen (e.g., the CDR). The antigen binding region can be derived from any animal species, such as rodents (e.g., rabbit, rat or hamster) and humans. In some embodiments, the antigen binding region will be of human origin.

The term “epitope” as used herein refers to a localized region on the surface of an antigen that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits a antibody response in an animal. An epitope having antigenic activity is a portion of a polypeptide to which an antibody immunospecifically binds as determined by any method well known in the art, for example, by the immunoassays described herein. Antigenic epitopes need not necessarily be immunogenic. Epitopes usually consist of chemically active surface groupings of molecules such as amino acids or sugar side chains and have specific three dimensional structural characteristics as well as specific charge characteristics. A region of a polypeptide contributing to an epitope may be contiguous amino acids of the polypeptide or the epitope may come together from two or more non-contiguous regions of the polypeptide. The epitope may or may not be a three-dimensional surface feature of the antigen.

The terms “fully human antibody” or “human antibody” are used interchangeably herein and refer to an antibody that comprises a human variable region and, in some embodiments, a human constant region. In specific embodiments, the terms refer to an antibody that comprises a variable region and constant region of human origin. The term “fully human antibody” includes antibodies having variable and constant regions corresponding to human germline immunoglobulin sequences as described by Kabat et al., Sequences of Proteins of Immunological Interest, Fifth Edition, U.S. Department of Health and Human Services, NIH Publication No. 91-3242, 1991.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences. See Kabat, E. A. et al. (1991) Sequences of Proteins of Immunological Interest, Fifth Edition, U.S. Department of Health and Human Services, NIH Publication No. 91-3242. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “monoclonal antibody” refers to an antibody obtained from a population of homogenous or substantially homogeneous antibodies, and each monoclonal antibody will typically recognize a single epitope on the antigen. In some embodiments, a “monoclonal antibody,” as used herein, is an antibody produced by a single hybridoma or other cell, wherein the antibody immunospecifically binds to only a epitope as determined, e.g., by ELISA or other antigen-binding or competitive binding assay known in the art or in the Examples provided herein. The term “monoclonal” is not limited to any particular method for making the antibody. For example, monoclonal antibodies provided herein may be made by the hybridoma method as described in Kohler et al.; Nature, 256:495 (1975) or may be isolated from phage libraries using the techniques as described herein, for example. Other methods for the preparation of clonal cell lines and of monoclonal antibodies expressed thereby are well known in the art. See, e.g., Chapter 11 in: Short Protocols in Molecular Biology, (2002) 5th Ed., Ausubel et al., eds., John Wiley and Sons, New York. Other exemplary methods of producing other monoclonal antibodies are provided in the Examples herein.

“Polyclonal antibodies” as used herein refers to an antibody population generated in an immunogenic response to a protein having many epitopes and thus includes a variety of different antibodies directed to the same and to different epitopes within the protein. Methods for producing polyclonal antibodies are known in the art. See, e.g., Chapter 11 in: Short Protocols in Molecular Biology, (2002) 5th Ed., Ausubel et al., eds., John Wiley and Sons, New York.

The term “expressed” or “expression” as used herein refers to the transcription from a gene to give an ribo nucleic acid molecule at least complementary in part to a region of one of the two nucleic acid strands of the gene. The term “expressed” or “expression” as used herein also refers to the translation from the RNA molecule to give a protein, a polypeptide or a portion thereof.

The term “level” refers to the amount, accumulation, or rate of a biomarker molecule. A level can be represented, for example, by the amount or the rate of synthesis of a messenger RNA (mRNA) encoded by a gene, the amount or the rate of synthesis of a polypeptide or protein encoded by a gene, or the amount or the rate of synthesis of a biological molecule accumulated in a cell or biological fluid. The term “level” refers to an absolute amount of a molecule in a sample or to a relative amount of the molecule, determined under steady-state or non-steady-state conditions.

An mRNA expressed at a higher level can be, for example, present at a level of about 5%, 10%, 20%, 30%, 40%, 50%, 60%, 70%, 90%, 100%, 200%, 300%, 500%, 1,000%, 5,000% or more of the comparative control mRNA level. An mRNA expressed at a lower level can be, for example, present at a level of about 99%, 95%, 90%, 80%, 70%, 60%, 50%, 40%, 30%, 20%, 10%, 1% or less of the comparative control mRNA level.

Similarly, a polypeptide or protein biomarker at a higher level can be, for example, present at a level of about 5%, 10%, 20%, 30%, 40%, 50%, 60%, 70%, 90%, 100%, 200%, 300%, 500%, 1,000%, 5,000% or more of the comparative control protein level. A polypeptide or protein biomarker expressed at a lower level can be, for example, present at a level of about 99%, 95%, 90%, 80%, 70%, 60%, 50%, 40%, 30%, 20%, 10%, 1% or less of the comparative control protein level.

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g. if they produce a given or detectable level of signal in a hybridization assay. Portions of polynucleotides are complementary to each other if they follow conventional base-pairing rules, e.g. A pairs with T (or U) and G pairs with C, although small regions (e.g. less than about 3 bases) of mismatch, insertion, or deleted sequence may be present.

“Sequence identity” or “identity” in the context of two nucleic acid sequences refers to the residues in the two sequences which are the same when aligned for maximum correspondence over a specified comparison window, and can take into consideration additions, deletions and substitutions.

The term “substantial identity” or “homologous” in their various grammatical forms in the context of polynucleotides generally means that a polynucleotide comprises a sequence that has a desired identity, for example, at least 60% identity, preferably at least 70% sequence identity, more preferably at least 80%, still more preferably at least 90% and even more preferably at least 95%, compared to a reference sequence. Another indication that nucleotide sequences are substantially identical is if two molecules hybridize to each other under stringent conditions.

The terms “isolated” and “purified” refer to isolation of a substance (such as mRNA, antibody or protein) such that the substance comprises a substantial portion of the sample in which it resides, i.e. greater than the substance is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample. For example, a sample of isolated mRNA can typically comprise at least about 1% total mRNA. Techniques for purifying polynucleotides are well known in the art and include, for example, gel electrophoresis, ion-exchange chromatography, affinity chromatography, flow sorting, and sedimentation according to density.

As used herein, the term “bound” can be used herein to indicate direct or indirect attachment. In the context of chemical structures, “bound” (or “bonded”) may refer to the existence of a chemical bond directly joining two moieties or indirectly joining two moieties (e.g., via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and also embodiments where the attachment is indirect.

The term “sample” as used herein relates to a material or mixture of materials, typically, although not necessarily, in fluid form, containing one or more components of interest.

“Biological sample” as used herein refers to a sample obtained from a biological subject, including sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, fractions and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include but are not limited to whole blood, partially purified blood, PBMCs, tissue biopsies, and the like.

The term “analyte” as used herein, refers to a known or unknown component of a sample.

The term “capture agent,” as used herein, refers to an agent that binds an mRNA or protein through an interaction that is sufficient to permit the agent to bind and concentrate the mRNA or protein from a homogeneous mixture.

The term “probe” as used herein, refers to a capture agent that is directed to a specific target mRNA biomarker sequence. Accordingly, each probe of a probe set has a respective target mRNA biomarker. A probe/target mRNA duplex is a structure formed by hybridizing a probe to its target mRNA biomarker.

The term “nucleic acid probe” or “oligonucleotide probe” refers to a nucleic acid capable of binding to a target nucleic acid of complementary sequence, such as the mRNA biomarkers provided herein, through one or more types of chemical bonds, usually through complementary base pairing, usually through hydrogen bond formation. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target mRNA biomarker of interest.

The term “stringent assay conditions” refers to conditions that are compatible to produce binding pairs of nucleic acids, e.g., probes and target mRNAs, of sufficient complementarity to provide for the desired level of specificity in the assay while being generally incompatible to the formation of binding pairs between binding members of insufficient complementarity to provide for the desired specificity. The term stringent assay conditions generally refer to the combination of hybridization and wash conditions.

A “label” or a “detectable moiety” in reference to a nucleic acid, refers to a composition that, when linked with a nucleic acid, renders the nucleic acid detectable, for example, by spectroscopic, photochemical, biochemical, immunochemical, or chemical means. Exemplary labels include, but are not limited to, radioactive isotopes, magnetic beads, metallic beads, colloidal particles, fluorescent dyes, enzymes, biotin, digoxigenin, haptens, and the like. A “labeled nucleic acid or oligonucleotide probe” is generally one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe can be detected by detecting the presence of the label bound to the nucleic acid or probe.

The terms “polymerase chain reaction,” or “PCR,” as used herein generally refers to a procedure wherein small amounts of a nucleic acid, RNA and/or DNA, are amplified as described, for example, in U.S. Pat. No. 4,683,195 to Mullis. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, N Y, 1989).

The term “cycle number” or “CT” when used herein in reference to PCR methods, refers to the PCR cycle number at which the fluorescence level passes a given set threshold level. The CT measurement can be used, for example, to approximate levels of mRNA in an original sample. The CT measurement is often used in terms of “dCT” or the “difference in the CT” score, when the CT of one nucleic acid is subtracted from the CT of another nucleic acid.

As used herein, and unless otherwise indicated, the term “optically pure” means a composition that comprises one optical isomer of a compound and is substantially free of other isomers of that compound. For example, an optically pure composition of a compound having one chiral center will be substantially free of the opposite enantiomer of the compound. An optically pure composition of a compound having two chiral centers will be substantially free of other diastereomers of the compound. A typical optically pure compound comprises greater than about 80% by weight of one enantiomer of the compound and less than about 20% by weight of other enantiomers of the compound, more preferably greater than about 90% by weight of one enantiomer of the compound and less than about 10% by weight of the other enantiomers of the compound, even more preferably greater than about 95% by weight of one enantiomer of the compound and less than about 5% by weight of the other enantiomers of the compound, more preferably greater than about 97% by weight of one enantiomer of the compound and less than about 3% by weight of the other enantiomers of the compound, and most preferably greater than about 99% by weight of one enantiomer of the compound and less than about 1% by weight of the other enantiomers of the compound.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “solvate” means a compound provided herein or a salt thereof, that further includes a stoichiometric or non-stoichiometric amount of solvent bound by non-covalent intermolecular forces. Where the solvent is water, the solvate is a hydrate.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center.

As used herein and unless otherwise indicated, the term “co-crystal” means a crystalline form that contains more than one compound in a crystal lattice. Co-crystals include crystalline molecular complexes of two or more non-volatile compounds bound together in a crystal lattice through non-ionic interactions. As used herein, co-crystals include pharmaceutical cocrystals wherein the crystalline molecular complexes containing a therapeutic compound and one or more additional non-volatile compound(s) (referred to herein as counter-molecule(s)). A counter-molecule in a pharmaceutical cocrystal is typically a non-toxic pharmaceutically acceptable molecule, such as, for example, food additives, preservatives, pharmaceutical excipients, or other APIs. In some embodiments, pharmaceutical cocrystals enhance certain physicochemical properties of drug products (e.g., solubility, dissolution rate, bioavailability and/or stability), without compromising the chemical structural integrity of the active pharmaceutical ingredient (API). See, e.g., Jones et al., “Pharmaceutical Cocrystals: An Emerging Approach to Physical Property Enhancement,” MRS Bulletin, 2006, 31, 875-879; Trask, “An Overview of Pharmaceutical Cocrystals as Intellectual Property,” Molecular Pharmaceutics, 2007, 4(3), 301-309; Schultheiss & Newman, “Pharmaceutical Cocrystals and Their Physicochemical Properties,” Crystal Growth & Design, 2009, 9(6), 2950-2967; Shan & Zaworotko, “The Role of Cocrystals in Pharmaceutical Science,” Drug Discovery Today, 2008, 13(9/10), 440-446; and Vishweshwar et al., “Pharmaceutical Co-Crystals,” J. Pharm. Sci., 2006, 95(3), 499-516.

A biological marker or “biomarker” is a substance whose detection indicates a particular biological state, such as, for example, the presence of cancer or malignant cells. In some embodiments, biomarkers can either be determined individually, or several biomarkers can be measured simultaneously.

In some embodiments, a “biomarker” indicates a change in the level of mRNA expression that may correlate with the risk or progression of a disease, or with the susceptibility of the disease to a given treatment. In some embodiments, the biomarker is a nucleic acid, such as a mRNA or cDNA. In some embodiments, the biomarker is a non-coding RNA, e.g., micro-RNA and mRNA-like non-coding RNA.

In additional embodiments, a “biomarker” indicates a change in the level of polypeptide or protein expression that may correlate with the risk, susceptibility to treatment, or progression of a disease. In some embodiments, the biomarker can be a polypeptide or protein, or a fragment thereof. The relative level of specific proteins can be determined by methods known in the art. For example, antibody based methods, such as an immunoblot, enzyme-linked immunosorbent assay (ELISA), or other methods can be used.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al. (1989) Molecular Cloning; A Laboratory Manual (2d ed.); D. N Glover, ed. (1985) DNA Cloning, Volumes I and II; M. J. Gait, ed. (1984) Oligonucleotide Synthesis; B. D. Hames & S J. Higgins, eds. (1984) Nucleic Acid Hybridization; B. D. Hames & S. J. Higgins, eds. (1984) Transcription and Translation; R. I. Freshney, ed. (1986) Animal Cell Culture; Immobilized Cells and Enzymes (IRL Press, 1986); Immunochemical Methods in Cell and Molecular Biology (Academic Press, London); Scopes (1987) Protein Purification: Principles and Practice (2d ed.; Springer Verlag, N.Y.); and D. M. Weir and C. C. Blackwell, eds. (1986) Handbook of Experimental Immunology, Volumes I-IV.

### 5.2 Methods of Using Biomarkers

The methods provided herein are based, in part, on the finding that the levels of certain proteins change differently in response to lenalidomide treatment as compared with Compound A treatment. Importantly, the set of differentially affected proteins by lenalidomide versus Compound A is different in different CLL patient groups. For example, as discussed in Section 6.3, the proteins which levels are differentially affected by Compound A and lenalidomide in CLL patients responsive to both lenalidomide and Compound A are listed in Table 1; the proteins which levels are differentially affected by Compound A and lenalidomide in CLL patients responsive better to Compound A than to lenalidomide are listed in Table 2; the proteins which levels are differentially affected by Compound A and lenalidomide in CLL patients responsive only to Compound A but not to lenalidomide are listed in Table 3; and the CLL proteins which levels are differentially affected by Compound A and lenalidomide in patients responsive to neither lenalidomide nor Compound A are listed in Table 4. Such differentially affected proteins can be used to associate a patient to a specific patient group, or as biomarkers for selecting a patient belonging to a specific patient group or predict a patient's response to compound treatment.

Accordingly, in one aspect, provided herein is a method of identifying a subject having CLL who is likely to be responsive to a treatment compound, comprising:

(a) obtaining a first sample and a second sample from the subject having CLL;

(b) administering Compound A to the first sample and administering lenalidomide to the second sample;

(c) determining the level of a biomarker in the first sample and determining the level of the biomarker in the second sample, wherein the biomarker is selected from the group consisting of biomarkers identified in Tables 1-3; and

(d) diagnosing the subject as being likely to be responsive to the treatment compound if the level of the biomarker in the first sample is different from the level of the biomarker in the second sample,

wherein the treatment compound is Compound A or lenalidomide.

In another aspect, provided herein is a method of predicting the responsiveness of a subject having or suspected of having CLL to a treatment compound, comprising:

(a) obtaining a first sample and a second sample from the subject;

(b) administering Compound A to the first sample and administering lenalidomide to the second sample;

(c) determining the level of a biomarker in the first sample and determining the level of the biomarker in the second sample, wherein the biomarker is selected from the group consisting of biomarkers identified in Tables 1-3; and

(d) diagnosing the subject as being likely to be responsive to the treatment compound if the level of the biomarker in the first sample is different from the level of the biomarker in the second sample,

wherein the treatment compound is Compound A or lenalidomide.

In another aspect, provided herein is a method of treating CLL in a subject, comprising:

(a) obtaining a first sample and a second sample from the subject;

(b) administering Compound A to the first sample and administering lenalidomide to the second sample;

(c) determining the level of a biomarker in the first sample and determining the level of the biomarker in the second sample, wherein the biomarker is selected from the group consisting of biomarkers identified in Tables 1-3;

(d) diagnosing the subject as being likely to be responsive to a treatment compound if the level of the biomarker in the first sample is different from the level of the biomarker in the second sample; and

(e) administering a therapeutically effective amount of the treatment compound to the subject diagnosed to be likely to be responsive to the treatment compound,

wherein the treatment compound is Compound A or lenalidomide.

As discussed in Section 6.3, the proteins which levels are differentially affected by Compound A and lenalidomide in patients responsive to both lenalidomide and Compound A (Case B1) are listed in Table 1, and include BCL2L1, GZMB, IGHM, IKZF1(isoform 1), IKZF1 (isoform 2), IRF8, KLF13, LGALS9, MARCKS, NDE1, NFKBIE, PTK2B, SAMSN1, SELL, SLAMF1, SNX20, SOD2, TCF7, TRAF1, WIZ, and ZBTB10.

Among these proteins in Table 1, BCL2L1, GZMB, IGHM, IRF8, KLF13, LGALS9, MARCKS, NDE1, NFKBIE, SNX20, TCF7, WIZ, and ZBTB10 are not differentially affected in other patient groups (cases), and thus can be unique markers for CLL patients that are responsive to both Compound A and lenalidomide treatment.

Thus, in some embodiments, the biomarker is selected from the group consisting of biomarkers identified in Table 1, and diagnosing the subject as being likely to be responsive to both Compound A and lenalidomide if the level of the biomarker in the first sample is different from the level of the biomarker in the second sample. In some embodiments, the biomarker is selected from the group consisting of BCL2L1, GZMB, IGHM, IRF8, KLF13, LGALS9, MARCKS, NDE1, NFKBIE, SNX20, TCF7, WIZ, and ZBTB10.

In some embodiments, the biomarker is selected from the group consisting of BCL2L1, MARCKS, NDE1, and ZBTB10, and diagnosing the subject as being likely to be responsive to both Compound A and lenalidomide if the level of the biomarker in the first sample is higher than the level of the biomarker in the second sample.

In other embodiments, the biomarker is selected from the group consisting of GZMB, IGHM, IRF8, KLF13, LGALS9, NFKBIE, SNX20, TCF7, and WIZ, and diagnosing the subject as being likely to be responsive to both Compound A and lenalidomide if the level of the biomarker in the first sample is lower than the level of the biomarker in the second sample.

In a specific embodiment, the biomarker is BCL2L1. In another specific embodiment, the biomarker is GZMB. In another specific embodiment, the biomarker is IGHM. In yet another embodiment, the biomarker is IRF8. In yet another embodiment, the biomarker is KLF13. In yet another embodiment, the biomarker is LGALS9. In yet another embodiment, the biomarker is MARCKS. In yet another embodiment, the biomarker is NDE1. In yet another embodiment, the biomarker is NFKBIE. In yet another embodiment, the biomarker is SNX20. In yet another embodiment, the biomarker is TCF7. In yet another embodiment, the biomarker is WIZ. In yet another embodiment, the biomarker is ZBTB10.

In some embodiments, the method provided herein comprises administering a therapeutically effective amount of Compound A or lenalidomide to the subject diagnosed to be likely to be responsive to both Compound A and lenalidomide.

As discussed in Section 6.3 below, the proteins which levels are differentially affected by Compound A and lenalidomide in CLL patients responsive better to Compound A than to lenalidomide (Case B2) are listed in Table 2, and include IKZF1 and IKZF3. The proteins which levels are differentially affected by Compound A and lenalidomide in patients responsive only to Compound A but not to lenalidomide (Case B3) are listed in Table 3 below, and include CD40, CR2, CTSS, GBP1, IKZF1, IKZF3, ISG20, PTK2B, SAMSN1, SASH1, SELL, SEMA7A, SLAMF1, SOD2, and TRAF1. Because Cases B2 and B3 are patients who respond to Compound A better than lenalidomide, differentially affected proteins identified only in Case B2 and B3 can be used to select patients responsive to Compound A, and these proteins include IKZF3, CD40, CR2, CTSS, GBP1, ISG20, SASH1, and SEMA7A.

Thus, in some embodiments, the biomarker is selected from the group consisting of biomarkers identified in Tables 2-3, and diagnosing the subject as being likely to be more responsive to Compound A than to lenalidomide if the level of the biomarker in the first sample is different from the level of the biomarker in the second sample.

In some embodiments, the biomarker is selected from the group consisting of IKZF3, CD40, CR2, CTSS, GBP1, ISG20, SASH1, and SEMA7A.

In some embodiments, the biomarker is selected from the group consisting of CD40, CR2, CTSS, GBP1, ISG20, SASH1, and SEMA7A, and diagnosing the subject as being likely to be more responsive to Compound A than to lenalidomide if the level of the biomarker in the first sample is higher than the level of the biomarker in the second sample. In a specific embodiment, the biomarker is CD40. In another specific embodiment, the biomarker is CR2. In another specific embodiment, the biomarker is CTSS. In yet another embodiment, the biomarker is GBP1. In yet another embodiment, the biomarker is ISG20. In yet another embodiment, the biomarker is SASH1. In yet another embodiment, the biomarker is SEMA7A.

In other embodiments, the biomarker is IKZF3, and diagnosing the subject as being likely to be more responsive to Compound A than to lenalidomide if the level of the biomarker in the first sample is lower than the level of the biomarker in the second sample.

In some embodiments, the method provided herein comprises administering a therapeutically effective amount of Compound A to the subject diagnosed to be likely to be more responsive to Compound A than to lenalidomide.

Since only patients of Case B4 do not respond to Compound A, the proteins differentially affected in Cases B1-B3 (as shown in Tables 1-3) can be used as biomarkers for identifying patients responsive to Compound A treatment.

Thus, in other embodiments, the biomarker is selected from the group consisting of biomarkers identified in Tables 1-3, diagnosing the subject as being likely to be responsive to Compound A if the level of the biomarker in the first sample is different from the level of the biomarker in the second sample.

In some embodiments, the biomarker is selected from the group consisting of BCL2L1, GZMB, IGHM, IKZF1, IRF8, KLF13, LGALS9, MARCKS, NDE1, NFKBIE, PTK2B, SAMSN1, SELL, SLAMF1, SNX20, SOD2, TCF7, TRAF1, WIZ, ZBTB10, IKZF3, CD40, CR2, CTSS, GBP1, ISG20, SASH1, and SEMA7A. In a specific embodiment, the biomarker is BCL2L1. In another specific embodiment, the biomarker is GZMB. In yet another specific embodiment, the biomarker is IGHM. In yet another specific embodiment, the biomarker is IKZF1. In yet another specific embodiment, the biomarker is IRF8. In yet another specific embodiment, the biomarker is KLF13. In yet another specific embodiment, the biomarker is LGALS9. In yet another specific embodiment, the biomarker is MARCKS. In yet another specific embodiment, the biomarker is NDE1. In yet another specific embodiment, the biomarker is NFKBIE. In yet another embodiment, the biomarker is PTK2B. In yet another specific embodiment, the biomarker is SAMSN1. In yet another specific embodiment, the biomarker is SELL. In yet another specific embodiment, the biomarker is SLAMF1. In yet another specific embodiment, the biomarker is SNX20. In yet another specific embodiment, the biomarker is SOD2. In yet another specific embodiment, the biomarker is TCF7. In yet another specific embodiment, the biomarker is TRAF1. In yet another specific embodiment, the biomarker is WIZ. In yet another specific embodiment, the biomarker is ZBTB10. In yet another specific embodiment, the biomarker is IKZF3. In yet another specific embodiment, the biomarker is CD40. In yet another specific embodiment, the biomarker is CR2. In yet another specific embodiment, the biomarker is CTSS. In yet another specific embodiment, the biomarker is GBP1. In yet another specific embodiment, the biomarker is ISG20. In yet another specific embodiment, the biomarker is SASH1. In yet another specific embodiment, the biomarker is SEMA7A.

In some embodiments, the biomarker is selected from the group consisting of PTK2B, SAMSN1, SLAMF1, SOD2, and TRAF1, and diagnosing the subject as being likely to be responsive to Compound A if the level of the biomarker in the first sample is higher than the level of the biomarker in the second sample. In a specific embodiment, the biomarker is PTK2B. In another specific embodiment, the biomarker is SAMSN1. In yet another embodiment, the biomarker is SLAMF1. In yet another embodiment, the biomarker is SOD2. In yet another embodiment, the biomarker is TRAF1.

In other embodiments, the biomarker is selected from the group consisting of IKZF1 and SELL, and diagnosing the subject as being likely to be responsive to Compound A if the level of the biomarker in the first sample is lower than the level of the biomarker in the second sample. In a specific embodiment, the biomarker is SELL. In another specific embodiment, the biomarker is IKZF1.

In some embodiments, the method provided herein comprises administering a therapeutically effective amount of Compound A to the subject diagnosed to be likely to be responsive to Compound A.

As discussed in Section 6.3 below, the level of PDE6D is differentially affected by Compound A and lenalidomide in patients responsive to neither lenalidomide nor Compound A, and PDE6D is not identified as differentially affected protein in other cases under the same conditions, and it thus can be used as a marker for patient not responsive to both lenalidomide and compound A.

Accordingly, in another aspect, provided herein is a method of identifying a subject having CLL who is not likely to be responsive to a treatment compound or predicting the responsiveness of a subject having or suspected of having CLL to a treatment compound, comprising:

(a) obtaining a first sample and a second sample from the subject;

(b) administering Compound A to the first sample and administering lenalidomide to the second sample;

(c) determining the level of a biomarker in the first sample and determining the level of the biomarker in the second sample, wherein the biomarker is PDE6D; and

(d) diagnosing the subject as not being likely to be responsive to the treatment compound if the level of the biomarker in the first sample is different from the level of the biomarker in the second sample,

wherein the treatment compound is Compound A or lenalidomide.

In some embodiments of the various methods provided herein, the level of the biomarker is determined by comparing to a reference level of the biomarker of a control sample, and wherein the control sample is obtained from the subject prior to administering Compound A or lenalidomide; and wherein the control sample is from the same source as the first and the second samples. In other embodiments of the various methods provided herein, the level of the biomarker is determined by comparing to a reference level of the biomarker of a control sample, and wherein the control sample is obtained from a healthy subject not having CLL; and wherein the control sample is from the same source as the first and the second samples.

In yet another aspect, the methods provided herein are based, in part, on the finding that the levels of certain proteins change differently in response to Compound A treatment in different patient groups. As discussed in Section 6.3 below, 41 proteins are identified as differentially impacted by Compound A in patients responsive to Compound A treatment versus patients not responsive to Compound A treatment, and these proteins are listed in Table 5. These proteins can also be used to select patients who are responsive to Compound A treatment.

Accordingly, in some embodiments, provided herein is a method of identifying a subject having CLL who is likely to be responsive to Compound A, comprising:

(a) obtaining a sample from the subject having CLL;

(b) administering Compound A to the sample;

(c) determining the level of a biomarker in the sample, wherein the biomarker is selected from the group consisting of biomarkers identified in Table 5; and

(d) diagnosing the subject as being likely to be responsive to Compound A if the level of the biomarker in the sample is different from a reference level of the biomarker in a control sample, wherein the control sample is obtained from a subject not responsive to Compound A.

In some embodiments, provided herein is a method of predicting the responsiveness of a subject having or suspected of having CLL to Compound A, comprising:

(a) obtaining a sample from the subject;

(b) administering Compound A to the sample;

(c) determining the level of a biomarker in the sample, wherein the biomarker is selected from the group consisting of biomarkers identified in Table 5; and

(d) diagnosing the subject as being likely to be responsive to Compound A if the level of the biomarker in the sample is different from a reference level of the biomarker in a control sample, wherein the control sample is obtained from a subject not responsive to Compound A.

In some embodiments, provided herein is a method of treating CLL in a subject, comprising:

(a) obtaining a sample from the subject having CLL;

(b) administering Compound A to the sample;

(c) determining the level of a biomarker in the sample, wherein the biomarker is selected from the group consisting of biomarkers identified in Table 5; and

(d) diagnosing the subject as being likely to be responsive to Compound A if the level of the biomarker in the sample is different from a reference level of the biomarker in a control sample, wherein the control sample is obtained from a subject not responsive to Compound A; and

(e) administering a therapeutically effective amount of Compound A to the subject diagnosed to be likely to be responsive to Compound A.

In some embodiments, the biomarker is selected from the group consisting of APOBEC3G, APOC3, APOL2, CR2, CTSS, FCHSD2, GBP2, GBP4, ICAM1, IDI1, IGHM, IKZF1, IKZF3, IL4I1, IRF5, IRF8, ISG20, KYNU, LAP3, LGALS9, NCF2, NCF4, NDE1, NECAP2, OAS1, PARP14, PDE6D, PLEK, PNP, PPA1, PPP1R18, RELB, SAMSN1, SEMA7A, SLFN5, SOD2, TAPBP, TNIP1, TRAF1, TRIP10, and ZFP91.

In a specific embodiment, the biomarker is APOBEC3G. In another specific embodiment, the biomarker is APOC3. In another specific embodiment, the biomarker is APOL2. In another specific embodiment, the biomarker is CR2. In another specific embodiment, the biomarker is CTSS. In another specific embodiment, the biomarker is FCHSD2. In another specific embodiment, the biomarker is GBP2. In another specific embodiment, the biomarker is GBP4. In another specific embodiment, the biomarker is ICAM1. In another specific embodiment, the biomarker is IDI1. In another specific embodiment, the biomarker is IGHM. In another specific embodiment, the biomarker is IKZF1. In another specific embodiment, the biomarker is IKZF3. In another specific embodiment, the biomarker is IL4I1. In another specific embodiment, the biomarker is IRF5. In another specific embodiment, the biomarker is IRF8. In another specific embodiment, the biomarker is ISG20. In another specific embodiment, the biomarker is KYNU. In another specific embodiment, the biomarker is LAP3. In another specific embodiment, the biomarker is LGALS9. In another specific embodiment, the biomarker is NCF2. In another specific embodiment, the biomarker is NCF4. In another specific embodiment, the biomarker is NDE1. In another specific embodiment, the biomarker is NECAP2. In another specific embodiment, the biomarker is OAS1. In another specific embodiment, the biomarker is PARP14. In another specific embodiment, the biomarker is PDE6D. In another specific embodiment, the biomarker is PLEK. In another specific embodiment, the biomarker is PNP. In another specific embodiment, the biomarker is PPA1. In another specific embodiment, the biomarker is PPP1R18. In another specific embodiment, the biomarker is RELB In another specific embodiment, the biomarker is SAMSN1. In another specific embodiment, the biomarker is SEMA7A. In another specific embodiment, the biomarker is SLFN5. In another specific embodiment, the biomarker is SOD2. In another specific embodiment, the biomarker is TAPBP. In another specific embodiment, the biomarker is TNIP1. In another specific embodiment, the biomarker is TRAF1. In another specific embodiment, the biomarker is TRIP10. In another specific embodiment, the biomarker is ZFP91.

As discussed in Section 6.3 below, effects of lenalidomide on protein levels were also analyzed and compared across four cases. For example, GZMB protein expression level increased upon lenalidomide exposure in Case B1 (where the patients respond to lenalidomide treatment) and decreased in Case B4 (where the patients do not respond to lenalidomide treatment), suggesting an association of this protein with lenalidomide resistance mechanisms. Thus, GZMB can be used to predict a patient's response to lenalidomide treatment or select patients responsive to lenalidomide treatment.

Accordingly, in some embodiments, provided herein is a method of identifying a subject having CLL who is likely to be responsive to lenalidomide, comprising:

(a) obtaining a sample from the subject having CLL;

(b) administering lenalidomide to the sample;

(c) determining the level of a biomarker in the sample, wherein the biomarker is GZMB, and

(d) diagnosing the subject as being likely to be responsive to lenalidomide if the level of the biomarker in the sample is different from a reference level of the biomarker in a control sample, wherein the control sample is obtained from a subject not responsive to lenalidomide.

In some embodiments, provided herein is a method of predicting the responsiveness of a subject having or suspected of having CLL to lenalidomide, comprising:

(a) obtaining a sample from the subject;

(b) administering lenalidomide to the sample;

(c) determining the level of a biomarker in the sample, wherein the biomarker is GZMB; and

(d) diagnosing the subject as being likely to be responsive to lenalidomide if the level of the biomarker in the sample is different from a reference level of the biomarker in a control sample, wherein the control sample is obtained from a subject not responsive to lenalidomide.

In some embodiments, provided herein is a method of treating CLL in a subject, comprising:

(a) obtaining a sample from the subject having CLL;

(b) administering lenalidomide to the sample;

(c) determining the level of a biomarker in the sample, wherein the biomarker is GZMB; and

(d) diagnosing the subject as being likely to be responsive to lenalidomide if the level of the biomarker in the sample is different from a reference level of the biomarker in a control sample, wherein the control sample is obtained from a subject not responsive to lenalidomide; and

(e) administering a therapeutically effective amount of lenalidomide to the subject diagnosed to be likely to be responsive to lenalidomide.

In yet another aspect, it is an interesting finding by the present application that changes of certain cellular pathways in response to Compound A treatment are highly associated with patients responsive to Compound A treatment. For example, upregulation of proteins in interferon signaling pathways in response to Compound A treatment is significantly associated with patients responsive to Compound A treatment.

Thus, in some embodiments, provided herein is a method of identifying a subject having CLL who is likely to be responsive to Compound A, comprising:

(a) obtaining a sample from the subject having CLL;

(b) administering Compound A to the sample;

(c) determining the level of a biomarker in the sample, wherein the biomarker is selected from the group consisting of proteins involved in interferon signaling pathway; and

(d) diagnosing the subject as being likely to be responsive to Compound A if the level of the biomarker in the sample is different from a reference level of the biomarker in a control sample, wherein the control sample is obtained from a subject not responsive to Compound A.

In some embodiments, provided herein is a method of predicting the responsiveness of a subject having or suspected of having CLL to Compound A, comprising:

(a) obtaining a sample from the subject;

(b) administering Compound A to the sample;

(c) determining the level of a biomarker in the sample, wherein the biomarker is selected from the group consisting of proteins involved in interferon signaling pathway; and

(d) diagnosing the subject as being likely to be responsive to Compound A if the level of the biomarker in the sample is different from a reference level of the biomarker in a control sample, wherein the control sample is obtained from a subject not responsive to Compound A.

In some embodiments, provided herein is a method of treating CLL in a subject, comprising:

(a) obtaining a sample from the subject having CLL;

(b) administering Compound A to the sample;

(c) determining the level of a biomarker in the sample, wherein the biomarker is selected from the group consisting of proteins involved in interferon signaling pathway; and

(d) diagnosing the subject as being likely to be responsive to Compound A if the level of the biomarker in the sample is different from a reference level of the biomarker in a control sample, wherein the control sample is obtained from a subject not responsive to Compound A; and

(e) administering a therapeutically effective amount of Compound A to the subject diagnosed to be likely to be responsive to Compound A.

In one aspect, provided herein is a method of identifying a subject having CLL who is likely to be responsive to a treatment compound, comprising:

(a) obtaining a sample from the subject;

(b) administering the treatment compound to the sample;

(c) determining the level of a biomarker in the sample, wherein the biomarker is PDE6D; and

(d) diagnosing the subject as being likely to be responsive to the treatment compound if the level of the biomarker in the sample is different from a reference level of the biomarker in a control sample, wherein the control sample is obtained from a subject not responsive to the treatment compound;

wherein the treatment compound is Compound A or lenalidomide.

In certain embodiments, the subject is identified as being likely to be responsive to the treatment compound if the level of the biomarker PDE6D in the sample is lower than the level of the biomarker in the control sample. In some embodiments, the treatment compound is Compound A. In other embodiments, the treatment compound is lenalidomide.

In another aspect, provided herein is a method of predicting the responsiveness of a subject having or suspected of having CLL to a treatment compound, comprising:

(a) obtaining a sample from the subject;

(b) administering the treatment compound to the sample;

(c) determining the level of a biomarker in the sample, wherein the biomarker is PDE6D; and

(d) diagnosing the subject as being likely to be responsive to the treatment compound if the level of the biomarker in the sample is different from a reference level of the biomarker in a control sample, wherein the control sample is obtained from a subject not responsive to the treatment compound;

wherein the treatment compound is Compound A or lenalidomide.

In certain embodiments, the subject is diagnosed as being likely to be responsive to the treatment compound if the level of the biomarker PDE6D in the sample is lower than the level of the biomarker in the control sample. In some embodiments, the treatment compound is Compound A. In other embodiments, the treatment compound is lenalidomide.

In yet another aspect, provided herein is a method of treating CLL in a subject, comprising:

(a) obtaining a sample from the subject;

(b) administering the treatment compound to the sample;

(c) determining the level of a biomarker in the sample, wherein the biomarker is PDE6D;

(d) diagnosing the subject as being likely to be responsive to the treatment compound if the level of the biomarker in the sample is different from a reference level of the biomarker in a control sample, wherein the control sample is obtained from a subject not responsive to the treatment compound; and

(e) administering a therapeutically effective amount of the treatment compound to the subject diagnosed to be likely to be responsive to the treatment compound;

wherein the treatment compound is Compound A or lenalidomide.

In certain embodiments, the subject is diagnosed as being likely to be responsive to the treatment compound if the level of the biomarker PDE6D in the sample is lower than the level of the biomarker in the control sample, and the treatment compound is administered. In some embodiments, the treatment compound is Compound A. In other embodiments, the treatment compound is lenalidomide.

In some embodiments of the various methods provided herein, the levels of two or more biomarkers provided herein are determined.

In some embodiments of the various methods provided herein, the administering a treatment compound to the sample from the subject is in vitro. In other embodiments, the administering a treatment compound to the sample from the subject is performed in vivo. In one embodiment, the samples are contacted with the compound for a period of time, e.g., 5, 10, 15, 20, 25, 30, 35, 40, 45, 50, or 55 minutes, or 1, 2, 3, 4, 5, 6, 7, 8, 9, 10, 11, 12, 13, 14, 15, 16, 17, 18, 19, 20, 21, 22, 23, or 24 hours, or 2 or 3 or more days.

In some embodiments of the various methods provided herein, the cancer is a leukemia. In some embodiments, the cancer is a lymphoma. In other embodiments, the cancer is a CLL. In other embodiments, the cancer is relapsed, refractory or resistant to conventional therapy. In other embodiments, the cancer is a relapsed or refracted CLL.

In some embodiments of the various methods provided herein, the treatment compound is an immunomodulatory compound. In some embodiments, the treatment compound is lenalidomide. In other embodiments, the treatment compound is Compound A.

In some embodiments, the level of the biomarker is measured by determining the protein level of the biomarker. In some embodiments, the methods provided herein comprise contacting proteins within the sample with a first antibody that immunospecifically binds to the biomarker protein. In some embodiments, the methods provided herein further comprise:

(i) contacting the biomarker protein bound to the first antibody with a second antibody with a detectable label, wherein the second antibody immunospecifically binds to the biomarker protein, and wherein the second antibody immunospecifically binds to a different epitope on the biomarker protein than the first antibody;

(ii) detecting the presence of the second antibody bound to the proteins; and

(iii) determining the amount of the biomarker protein based on the amount of the detectable label in the second antibody.

In other embodiments, the methods provided herein further comprise:

(i) contacting the biomarker protein bound to the first antibody with a second antibody with a detectable label, wherein the second antibody immunospecifically binds to the first antibody;

(ii) detecting the presence of the second antibody bound to the proteins; and

(iii) determining the amount of the biomarker protein based on the amount of the detectable label in the second antibody.

In other embodiments, the level of the biomarker is measured by determining the mRNA level of the biomarker. In yet other embodiments, the level of the biomarker is measured by determining the cDNA level of the biomarker. In some embodiments, the level of the biomarker is measured using quantitative PCR (qPCR).

In some embodiments of the various methods provided herein, the patients have been previously treated for cancer but are non-responsive to standard therapies, as well as those who have not previously been treated. The present disclosure also encompasses methods of treating patients regardless of patient's age, although some diseases or disorders are more common in certain age groups. The application further encompasses methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

In certain embodiments of the various methods provided herein, a therapeutically or prophylactically effective amount of lenalidomide or Compound A is administered to the subject. A therapeutically or prophylactically effective amount of lenalidomide or Compound A is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, or from about 0.05 to about 10 mg per day.

In certain embodiments, the therapeutically or prophylactically effective amount of lenalidomide or Compound A is about 0.1, about 0.2, about 0.5, about 1, about 2, about 5, about 10, about 15, about 20, about 25, about 30, about 40, about 45, about 50, about 60, about 70, about 80, about 90, about 100, or about 150 mg per day.

In one embodiment, the recommended daily dose range of lenalidomide, Compound A, or an enantiomer or a mixture of enantiomers thereof, or a pharmaceutically acceptable salt, solvate, hydrate, co-crystal, clathrate, or polymorph thereof, for the conditions described herein lie within the range of from about 0.5 mg to about 50 mg per day, preferably given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the compound can be administered in an amount of about 25 mg/day to patients with leukemia, e.g., CLL. In a particular embodiment, the compound can be administered in an amount of about 10 mg/day to patients with leukemia including CLL.

In certain embodiments, the therapeutically or prophylactically effective amount of lenalidomide or Compound A is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, or from about 0.01 to about 1 mg/kg/day.

The administered dose can also be expressed in units other than mg/kg/day. For example, doses for parenteral administration can be expressed as mg/m2/day. One of ordinary skill in the art would readily know how to convert doses from mg/kg/day to mg/m2/day to given either the height or weight of a subject or both (see, www.fda.gov/cder/cancer/animalframe.htm). For example, a dose of 1 mg/kg/day for a 65 kg human is approximately equal to 38 mg/m2/day.

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein, e.g., lenalidomide, Compound A, or an enantiomer or a mixture of enantiomers thereof, or a pharmaceutically acceptable salt, solvate, hydrate, co-crystal, clathrate, or polymorph thereof. Once steady state is reached, there are minor peaks and troughs on the time dependent curve of the plasma concentration of the compound.

In certain embodiments, the amount of the compound administered is sufficient to provide a maximum plasma concentration (peak concentration) of the compound, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In certain embodiments, the amount of the compound administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

Depending on the stage of the disease to be treated and the subject's condition, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, may be formulated, alone or together, in suitable dosage unit with pharmaceutically acceptable excipients, carriers, adjuvants and vehicles, appropriate for each route of administration.

In one embodiment, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered orally. In another embodiment, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered parenterally. In yet another embodiment, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered intravenously.

The compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, can be delivered as a single dose such as, e.g., a single bolus injection, or oral tablets or pills; or over time, such as, e.g., continuous infusion over time or divided bolus doses over time. The compound can be administered repeatedly if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205-216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as lenalidomide, is administered once or more than once each day, for example, for a period of time. The term “continuous” is intended to mean that a therapeutic compound, such as lenalidomide or Compound A, is administered daily for an uninterrupted period of at least 10 days to 52 weeks. The term “intermittent” or “intermittently” as used herein is intended to mean stopping and starting at either regular or irregular intervals. For example, intermittent administration of the compound provided herein, e.g., lenalidomide or Compound A, is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as lenalidomide, is administered daily or continuously but with a rest period.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered once a day. In another embodiment, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered twice a day. In yet another embodiment, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered three times a day. In still another embodiment, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered four times a day.

In certain embodiments, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered once per day from one day to six months, from one week to three months, from one week to four weeks, from one week to three weeks, or from one week to two weeks. In certain embodiments, the compound provided herein, e.g., lenalidomide, Compound A, or a pharmaceutically acceptable salt or solvate thereof, is administered once per day for one week, two weeks, three weeks, or four weeks. In one embodiment, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered once per day for one week. In another embodiment, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered once per day for two weeks. In yet another embodiment, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered once per day for three weeks. In still another embodiment, the compound provided herein, e.g., lenalidomide, Compound A, or an enantiomer or a mixture of enantiomers thereof; or a pharmaceutically acceptable salt, solvate, hydrate, co-crystal, clathrate, or polymorph thereof, is administered once per day for four weeks.

In some embodiments of the various methods provided herein, the method further comprises administering a therapeutically effective amount of another second active agent or a support care therapy. Second active agents can be large molecules (e.g., proteins) or small molecules (e.g., synthetic inorganic, organometallic, or organic molecules). In some embodiments, the other second active agent is a therapeutic antibody that specifically binds to a cancer antigen, hematopoietic growth factor, cytokine, anti-cancer agent, antibiotic, cox-2 inhibitor, immunomodulatory agent, immunosuppressive agent, corticosteroid or a pharmacologically active mutant or derivative thereof.

In some embodiments, the second active agents are small molecules that can alleviate adverse effects associated with the administration of a compound provided herein or an enantiomer or a mixture of enantiomers thereof, or a pharmaceutically acceptable salt, solvate, hydrate, co-crystal, clathrate, or polymorph thereof. However, like some large molecules, many are believed to be capable of providing a synergistic effect when administered with (e.g., before, after or simultaneously) a compound provided herein or an enantiomer or a mixture of enantiomers thereof, or a pharmaceutically acceptable salt, solvate, hydrate, co-crystal, clathrate, or polymorph thereof. Examples of small molecule second active agents include, but are not limited to, anti-cancer agents, antibiotics, immunosuppressive agents, and steroids.

### 5.3 Methods of Detecting and Quantifying Biomarkers

In certain embodiments, provided herein are methods of detecting and quantifying the protein level of a biomarker provided herein from a biological sample, contacting proteins within the sample with a first antibody that immunospecifically binds to the biomarker protein. In some embodiments, the methods provided herein further comprise (i) contacting the biomarker protein bound to the first antibody with a second antibody with a detectable label, wherein the second antibody immunospecifically binds to the biomarker protein, and wherein the second antibody immunospecifically binds to a different epitope on the biomarker protein than the first antibody; (ii) detecting the presence of second antibody bound to the proteins; and (iii) determining the amount of the biomarker protein based on the amount of detectable label in the second antibody. In other embodiments, the methods provided herein further comprise (i) contacting the biomarker protein bound to the first antibody with a second antibody with a detectable label, wherein the second antibody immunospecifically binds to the first antibody; (ii) detecting the presence of second antibody bound to the proteins; and (iii) determining the amount of the biomarker protein based on the amount of detectable label in the second antibody.

In some embodiments of the various methods provided herein, the method comprises using dual staining immunohistochemistry to determine the level of one or more biomarkers. In a dual staining immunohistochemistry assay, a first biomarker provided herein and a second biomarker are simultaneously detected using a first labeled antibody targeting a first biomarker provided herein and a second labeled antibody targeting a second biomarker.

Thus, in some embodiments, the method provided herein comprises (i) contacting proteins within a sample with a first antibody that immunospecifically binds to a first biomarker provided herein, the first antibody being coupled with a first detectable label; (ii) contacting the proteins within the sample with a second antibody that immunospecifically binds to a second biomarker, the second antibody being coupled with a second detectable label; (iii) detecting the presence of the first antibody and the second antibody bound to the proteins; and (iv) determining the levels of the two biomarkers provided herein based on the amount of detectable label in the first antibody and the second antibody, and determining the ratio of the levels of the two biomarkers.

In certain embodiments, provided herein are methods of detecting and quantifying the RNA (e.g., mRNA) level of a biomarker provided herein from a biological sample, comprising: (a) obtaining RNA from the sample; (b) contacting the RNA with a primer comprising a sequence specifically binding to a sequence in the RNA to generate a first DNA molecule having a sequence complementary to said RNA; (c) amplifying the DNA corresponding to a segment of a gene encoding the biomarker; and (d) determining the RNA level of the biomarker based on the amount of the amplified DNA.

In certain embodiments of the various methods provided herein, the two or more of the steps are performed sequentially. In other embodiments of the methods provided herein, two or more of steps are performed in parallel (e.g., at the same time).

Exemplary assays provided herein for the methods of detecting and quantifying the protein level of a biomarker, are immunoassays, such as western blot analysis, and an enzyme-linked immunosorbent assay (ELISA) (e.g., a sandwich ELISA). An exemplary assay provided herein for the methods of detecting and quantifying the RNA level of a biomarker provided herein, or a combination thereof, is reverse transcription polymerase chain reaction (RT-PCR), e.g., quantitative PCR or qPCR.

5.3.1 Methods of Detecting mRNA Levels in a Sample

Several methods of detecting or quantitating mRNA levels are known in the art and are suitable for use in the methods provided herein for measuring the level of the biomarker. Exemplary methods include, but are not limited to, northern blots, ribonuclease protection assays, and PCR-based methods. When the biomarker is an mRNA molecule, the mRNA sequence, or a fragment thereof, can be used to prepare a probe that is at least partially complementary. The probe can then be used to detect the mRNA sequence in a sample, using any suitable assay, such as PCR-based methods, Northern blotting, or a dipstick assay.

The assay method can be varied depending on the type of mRNA information desired. Exemplary methods include, but are not limited to, Northern blots and PCR-based methods (e.g., qRT-PCR). Methods such as qRT-PCR can also accurately quantitate the amount of the mRNA in a sample.

Any suitable assay platform can be used to determine the presence of the mRNA in a sample. For example, an assay may be in the form of a dipstick, a membrane, a chip, a disk, a test strip, a filter, a microsphere, a slide, a multiwell plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). In certain embodiments, the sample is labeled with fluorescent label. Exemplary fluorescent dyes include, but are not limited to, xanthene dyes, fluorescein dyes, rhodamine dyes, fluorescein isothiocyanate (FITC), 6-carboxyfluorescein (FAM), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine 6G (R6G5 or G5), 6-carboxyrhodamine 6G (R6G6 or G6), and rhodamine 110; cyanine dyes, e.g. Cy3, Cy5 and Cy7 dyes; Alexa dyes, e.g. Alexa-fluor-555; coumarin, diethylaminocoumarin, umbelliferone; benzimide dyes, e.g. Hoechst 33258; phenanthridine dyes, e.g., Texas red; ethidium dyes; acridine dyes; carbazole dyes; phenoxazine dyes; porphyrin dyes; polymethine dyes, BODIPY dyes, quinoline dyes, pyrene, fluorescein chlorotriazinyl, R110, Eosin, JOE, R6G, tetramethylrhodamine, lissamine, ROX, and napthofluorescein.

The nucleic acids may be present in specific, addressable locations on a solid support; each corresponding to at least a portion of mRNA sequences of a biomarker.

In certain embodiments, an mRNA assay comprises the steps of 1) obtaining surface-bound probes for one or more biomarkers; 2) hybridizing a population of mRNAs to the surface-bound probes under conditions sufficient to provide for specific binding; (3) removing unbound nucleic acids in the hybridization step; and (4) detecting the hybridized mRNAs.

Hybridization can be carried out under suitable hybridization conditions, which may vary in stringency as desired. Typical conditions are sufficient to produce probe/target complexes on a solid surface between complementary binding members, i.e., between surface-bound probes and complementary mRNAs in a sample.

In certain embodiments, stringent hybridization conditions are used. Standard hybridization techniques (e.g., under conditions sufficient to provide for specific binding of target mRNAs in the sample to the probes) are described in Kallioniemi et al., Science 258:818-821 (1992) and WO 93/18186, the disclosure of each which is incorporated herein by reference in its entirety. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al. Meth. Enzymol., 21:470-480 (1981); and Angerer et al. in Genetic Engineering: Principles and Methods (Setlow and Hollaender, Eds.) Vol 7, pages 43-65 (Plenum Press, New York 1985). Selection of appropriate conditions, including temperature, salt concentration, polynucleotide concentration, hybridization time, and stringency of washing conditions, depends on experimental design, including the source of a sample, the identity of capture agents, the degree of complementarity expected, etc., and may be determined as a matter of routine experimentation for those of ordinary skill in the art.

After the mRNA hybridization procedure, the surface bound polynucleotides are washed to remove unbound nucleic acids. Washing may be performed using any convenient washing protocol. In certain embodiments, the washing conditions are stringent. The hybridization of the target mRNAs to the probes is then detected using standard techniques.

In certain embodiments, the mRNA level of a biomarker is determined using a PCR-based method. Examples of PCR assays can be found in U.S. Pat. No. 6,927,024, the disclosure of which is incorporated by reference herein in its entirety. Examples of RT-PCR methods can be found in U.S. Pat. No. 7,122,799, the disclosure of which is incorporated by reference herein in its entirety. Examples of fluorescent in situ PCR methods can be found in U.S. Pat. No. 7,186,507, the disclosure of which is incorporated by reference herein in its entirety. In a specific embodiment, Roche Light Cycler 480 and UPL-RT-PCR system are used to quantitate mRNA levels using G6PD level as internal control.

In certain embodiments, quantitative real-time reverse transcription-PCR (qRT-PCR) is used for both the detection and quantification of mRNAs (Bustin, et al., Clin. Sci., 2005, 109, 365-379). Quantitative results obtained by qRT-PCR are generally more informative than qualitative data. Examples of qRT-PCR-based methods can be found in U.S. Pat. No. 7,101,663, the disclosure of which is incorporated by reference herein in its entirety.

In contrast to regular reverse transcriptase-PCR and analysis by agarose gels, real-time PCR gives quantitative results. An additional advantage of real-time PCR is the relative ease and convenience of use. Instruments for real-time PCR, such as Applied Biosystems 7500, are available commercially. The reagents for real-time PCR, such as TaqMan Sequence Detection chemistry, are also commercially available.

To determine the cycle number at which the fluorescence signal associated with a particular amplicon accumulation crosses the threshold (referred to as CT), the data can be analyzed, for example, using a 7500 Real-Time PCR System Sequence Detection software v1.3, using the comparative CT relative quantification calculation method. Using this method, the output is expressed as a fold-change in expression levels. In some embodiments, the threshold level can be selected to be automatically determined by the software. In some embodiments, the threshold level is set to be above the baseline, but sufficiently low to be within the exponential growth region of an amplification curve.

Techniques known to one skilled in the art may be used to measure the amount of an RNA transcript(s). In some embodiments, the amount of one, two, three, four, five or more RNA transcripts is measured using deep sequencing, such as ILLUMINA® RNASeq, ILLUMINA® next generation sequencing (NGS), ION TORRENT™ RNA next generation sequencing, 454™ pyrosequencing, or Sequencing by Oligo Ligation Detection (SOLID). In other embodiments, the amount of multiple RNA transcripts is measured using a microarray and/or gene chip. In certain embodiments, the amount of one, two, three or more RNA transcripts is determined by RT-PCR. In other embodiments, the amount of one, two, three or more RNA transcripts is measured by RT-qPCR. Techniques for conducting these assays are known to one skilled in the art.

In some embodiments, a statistical analysis or other analysis is performed on data from the assay utilized to measure an RNA transcript or protein. In certain specific embodiments, p value of those RNA transcripts or proteins differentially expressed is 0.1, 0.5, 0.4, 0.3, 0.2, 0.01, 0.05, 0.001, 0.005, or 0.0001. In specific embodiments, a false discovery rate (FDR) of 10%, 9%, 8%, 7%, 6%, 5%, 4%, 3%, 2%, 1% or less is selected.

5.3.2 Methods of Detecting Polypeptide or Protein Biomarkers

When the biomarker is a protein, polypeptide, or peptide, several protein detection and quantitation methods can be used to measure the level of the biomarker. Any suitable protein quantitation method can be used in the methods provided herein. In certain embodiments, antibody-based methods are used. Exemplary methods that can be used include, but are not limited to, immunoblotting (western blot), enzyme-linked immunosorbent assay (ELISA), immunohistochemistry, flow cytometry, cytometric bead array, and mass spectroscopy. In certain embodiments, a biomarker protein is detected using mass spectroscopy. Several types of ELISA are commonly used, including direct ELISA, indirect ELISA, and sandwich ELISA.

### 5.4 Subjects and Samples

In certain embodiments, the various methods provided herein use samples (e.g., biological samples) from subjects or individuals (e.g., patients). The subject can be a patient, such as, a patient with a cancer (e.g., CLL). The subject can be a mammal, for example, a human. The subject can be male or female, and can be an adult, child or infant. Samples can be analyzed at a time during an active phase of a cancer (e.g., CLL), or when the cancer (e.g., CLL) is inactive. In certain embodiments, more than one sample from a subject can be obtained.

In certain embodiments, the sample used in the methods provided herein comprises body fluids from a subject. Non-limiting examples of body fluids include blood (e.g., peripheral whole blood, peripheral blood), blood plasma, amniotic fluid, aqueous humor, bile, cerumen, cowper's fluid, pre-ejaculatory fluid, chyle, chyme, female ejaculate, interstitial fluid, lymph, menses, breast milk, mucus, pleural fluid, pus, saliva, sebum, semen, serum, sweat, tears, urine, vaginal lubrication, vomit, water, feces, internal body fluids, including cerebrospinal fluid surrounding the brain and the spinal cord, synovial fluid surrounding bone joints, intracellular fluid is the fluid inside cells, and vitreous humour the fluids in the eyeball. In some embodiments, the sample is a blood sample. The blood sample can be obtained using conventional techniques as described in, e.g. Innis et al, editors, PCR Protocols (Academic Press, 1990). White blood cells can be separated from blood samples using convention techniques or commercially available kits, e.g. RosetteSep kit (Stein Cell Technologies, Vancouver, Canada). Sub-populations of white blood cells, e.g. mononuclear cells, B cells, T cells, monocytes, granulocytes or lymphocytes, can be further isolated using conventional techniques, e.g. magnetically activated cell sorting (MACS) (Miltenyi Biotec, Auburn, Calif.) or fluorescently activated cell sorting (FACS) (Becton Dickinson, San Jose, Calif.).

In one embodiment, the blood sample is from about 0.1 mL to about 10.0 mL, from about 0.2 mL to about 7 mL, from about 0.3 mL to about 5 mL, from about 0.4 mL to about 3.5 mL, or from about 0.5 mL to about 3 mL. In another embodiment, the blood sample is about 0.3, 0.4, 0.5, 0.6, 0.7, 0.8, 0.9, 1.0, 1.5, 2.0, 2.5, 3.0, 3.5, 4.0, 4.5, 5.0, 6.0, 7.0, 8.0, 9.0 or 10.0 mL.

In some embodiments, the sample used in the present methods comprises a biopsy (e.g., a tumor biopsy). The biopsy can be from any organ or tissue, for example, skin, liver, lung, heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy.

In one embodiment, the sample used in the methods provided herein is obtained from the subject prior to the subject receiving a treatment for the disease or disorder. In another embodiment, the sample is obtained from the subject during the subject receiving a treatment for the disease or disorder. In another embodiment, the sample is obtained from the subject after the subject receiving a treatment for the disease or disorder. In various embodiments, the treatment comprises administering a compound (e.g., a compound provided below) to the subject.

### 5.5 Compounds

In some embodiments, the treatment compound is an immunomodulatory compound. In one embodiment, the treatment compounds encompass those immunomodulatory compounds from Celgene Corporation.

As used herein and unless otherwise indicated, the term “immunomodulatory compound” encompasses certain small organic molecules that inhibit LPS induced monocyte TNF-α, IL-1B, IL-12, IL-6, MIP-1α, MCP-1, GM-CSF, G-CSF, and COX-2 production. Specific immunomodulatory compounds are provided herein.

TNF-α is an inflammatory cytokine produced by macrophages and monocytes during acute inflammation. TNF-α is responsible for a diverse range of signaling events within cells. Without being limited by a particular theory, one of the biological effects exerted by the immunomodulatory compounds provided herein is the reduction of myeloid cell TNF-α production. In certain embodiments, the immunomodulatory compounds provided herein enhance the degradation of TNF-α mRNA.

Various immunomodulatory compounds provided herein contain one or more chiral centers, and can exist as mixtures of enantiomers (e.g., racemic mixtures) or mixtures of diastereomers. The methods provided herein encompass the use of stereomerically pure forms of such compounds as well as mixtures of those forms. For example, mixtures comprising equal or unequal amounts of the enantiomers of a particular immunomodulatory compound may be used in methods provided herein. These isomers may be asymmetrically synthesized or resolved using standard techniques, such as chiral columns or chiral resolving agents. See, Jacques et al., Enantiomers, Racemates and Resolutions (Wiley-Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (Eliel, Ed., Univ. of Notre Dame Press, Notre Dame, Ind., 1972).

In certain embodiments, the immunomodulatory compound has the structure of Formula I:

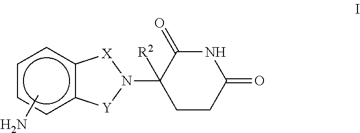

wherein one of X and Y is C═O, the other of X and Y is C═O or CH₂, and R² is hydrogen or lower alkyl, in one embodiment, methyl.

In certain embodiments, the immunomodulatory compound is:

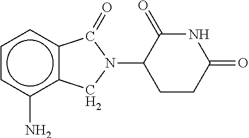

1-oxo-2-(2,6-dioxopiperidin-3-yl)-4-aminoisoindoline (lenalidomide); or a optically pure isomer thereof. The immunomodulatory compounds can be obtained via standard, synthetic methods. See U.S. Pat. No. 5,635,517, the disclosure of which is incorporated herein by reference in its entirety. The immunomodulatory compounds are also available from Celgene Corporation, Warren, N.J.

In certain embodiments, the treatment compound is a compound having the structure of Formula II:

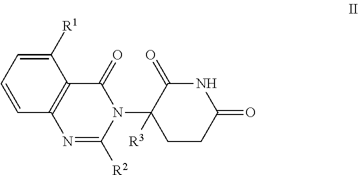

or a pharmaceutically acceptable salt, solvate, or stereoisomer thereof, wherein:
R¹ is:
   hydrogen;
   halo;
   —(CH₂)—OH;
   C₁₋₆ alkyl, optionally substituted with one or more halo;
   C₁₋₆ alkoxy, optionally substituted with one or more halo; or
   —(CH₂)ₙNHR^{a}, wherein R^{a} is:
      hydrogen;
      C₁₋₆ alkyl, optionally substituted with one or more halo;
      —(CH₂)ₙ-(6 to 10 membered aryl);
      —C(O)—(CH₂)ₙ-(6 to 10 membered aryl) or —C(O)—(CH₂)ₙ-(5 to 10 membered heteroaryl), wherein the aryl or heteroaryl is optionally substituted with one or more of: halo; —SCF₃; C₁₋₆ alkyl, itself optionally substituted with one or more halo; or C₁₋₆ alkoxy, itself optionally substituted with one or more halo;
      —C(O)—C₁₋₈ alkyl, wherein the alkyl is optionally substituted with one or more halo;
      —C(O)—(CH₂)ₙ—(C₃-C₁₀-cycloalkyl);
      —C(O)—(CH₂)ₙ—NR^{b}R^{c}, wherein R^{b} and R^{c} are each independently:
         hydrogen;
         C₁₋₆ alkyl, optionally substituted with one or more halo;
         C₁₋₆ alkoxy, optionally substituted with one or more halo; or
         6 to 10 membered aryl, optionally substituted with one or more of: halo; C₁₋₆ alkyl, itself optionally substituted with one or more halo; or C₁₋₆ alkoxy, itself optionally substituted with one or more halo;
      —C(O)—(CH₂)ₙ—O—C₁₋₆ alkyl; or
      —C(O)—(CH₂)ₙ—O—(CH₂)ₙ-(6 to 10 membered aryl);
R² is: hydrogen; —(CH₂)OH; phenyl; —O—C₁₋₆ alkyl; or C₁₋₆ alkyl, optionally substituted with one or more halo;
R³ is: hydrogen; or C₁₋₆ alkyl, optionally substituted with one or more halo; and
n is 0, 1, or 2.

In certain embodiments, the treatment compound is a compound of Formula III:

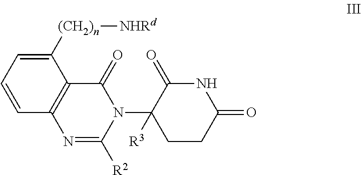

or a pharmaceutically acceptable salt, solvate, or stereoisomer thereof, wherein:
R^{d} is: hydrogen;
   C₁₋₆ alkyl, optionally substituted with one or more halo;
   —C(O)—C₁₋₈ alkyl, wherein the alkyl is optionally substituted with one or more halo;
   —C(O)—(CH₂)ₙ—C₃₋₁₀ cycloalkyl;
   —C(O)—(CH₂)ₙ—NR^{e}R^{f}, wherein R^{e} and R^{f} are each independently:
      hydrogen;
      C₁₋₆ alkyl, optionally substituted with one or more halo; or
      C₁₋₆ alkoxy, optionally substituted with one or more halo; or
   —C(O)—(CH₂)ₙ—O—C₁₋₆ alkyl.
R⁷ is: hydrogen; —(CH₂)—OH; phenyl; —O—C₁₋₆ alkyl; or C₁₋₆ alkyl, optionally substituted with one or more halo;
R⁸ is: hydrogen; or C₁₋₆ alkyl, optionally substituted with one or more halo; and
n is 0, 1, or 2.

In certain embodiments, the treatment compound is

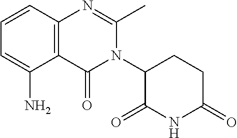 [SMILES: Cc1nc2cccc(N)c2c(=O)n1C1CCC(=O)NC1=O]

3-(5-amino-2-methyl-4-oxo-4H-quinazolin-3-yl)-piperidine-2,6-dione (Compound A), or a stereoisomer thereof, or a pharmaceutically acceptable salt, solvate, hydrate, co-crystal, clathrate, or a polymorph thereof.

All of the compounds described herein can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

Compounds provided herein may be small organic molecules having a molecular weight less than about 1,000 g/mol, and are not proteins, peptides, oligonucleotides, oligosaccharides or other macromolecules.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.6 Pharmaceutical Compositions

In certain embodiments, provided herein are pharmaceutical compositions comprising a compound provided herein, e.g., lenalidomide or Compound A. The pharmaceutical compositions provided herein contain therapeutically effective amounts of one or more of compounds provided herein and a pharmaceutically acceptable carrier, diluent or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of cancer, including solid tumors and blood borne tumors.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of cancer, including solid tumors and blood borne tumors.

In certain embodiments, a therapeutically effective dosage should produce a serum concentration of active ingredient of from about 0.1 ng/ml to about 50-100 μg/ml. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to orally, parenterally, rectally, topically and locally. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non toxic carrier may be prepared. For oral administration, a pharmaceutically acceptable non toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001%-100% active ingredient, in certain embodiments, about 0.1%-85% or about 75%-95%.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

5.6.1 Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non effervescent granules and effervescent preparations reconstituted from effervescent granules. Aqueous solutions include, for example, elixirs and syrups. Emulsions are either oil in-water or water in oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non aqueous liquids, emulsifying agents and preservatives. Suspensions use pharmaceutically acceptable suspending agents and preservatives. Pharmaceutically acceptable substances used in non effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

5.6.2 Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

5.6.3 Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

5.6.4 Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5.6.5 Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

5.6.6 Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and U.S. Pat. Nos. 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

5.6.7 Targeted Formulations

The compounds provided herein, or pharmaceutically acceptable salts thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

5.6.8 Articles of Manufacture

The compounds or pharmaceutically acceptable salts can be packaged as articles of manufacture containing packaging material, a compound or pharmaceutically acceptable salt thereof provided herein, which is used for treatment, prevention or amelioration of one or more symptoms or progression of cancer, including solid tumors and blood borne tumors, and a label that indicates that the compound or pharmaceutically acceptable salt thereof is used for treatment, prevention or amelioration of one or more symptoms or progression of cancer, including solid tumors and blood borne tumors.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

### 5.7 Kits for Detecting Biomarker Levels

In certain embodiments, provided herein is a kit for detecting the mRNA level of one or more biomarkers. In certain embodiments, the kit comprises one or more probes that bind specifically to the mRNAs of the one or more biomarkers. In certain embodiments, the kit further comprises a washing solution. In certain embodiments, the kit further comprises reagents for performing a hybridization assay, mRNA isolation or purification means, detection means, as well as positive and negative controls. In certain embodiments, the kit further comprises an instruction for using the kit. The kit can be tailored for in-home use, clinical use, or research use.

In certain embodiments, provided herein is a kit for detecting the protein level of one or more biomarkers. In certain embodiments, the kits comprises a dipstick coated with an antibody that recognizes the protein biomarker, washing solutions, reagents for performing the assay, protein isolation or purification means, detection means, as well as positive and negative controls. In certain embodiments, the kit further comprises an instruction for using the kit. The kit can be tailored for in-home use, clinical use, or research use.

Such a kit may employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an oral tissue, gastrointestinal tissue, an organ, an organelle, a biological fluid, a blood sample, a urine sample, or a skin sample. The biological sample can be, for example, a lymph node biopsy, a bone marrow biopsy, or a sample of peripheral blood tumor cells.

In another embodiment, the kit comprises a solid support, nucleic acids contacting the support, where the nucleic acids are complementary to at least 20, 50, 100, 200, 350, or more bases of mRNA, and a means for detecting the expression of the mRNA in a biological sample.

In a specific embodiment, the pharmaceutical or assay kit comprises, in a container, a compound or a pharmaceutical composition thereof, and further comprises, in one or more containers, components for isolating RNA. In another specific embodiment, the pharmaceutical or assay kit comprises, in a container, a compound or a pharmaceutical composition, and further comprises, in one or more containers, components for conducting RT-PCR, RT-qPCR, deep sequencing or a microarray. In some embodiments, the kit comprises a solid support, nucleic acids contacting the support, where the nucleic acids are complementary to at least 20, 50, 100, 200, 350, or more bases of mRNA, and a means for detecting the expression of the mRNA in a biological sample.

In certain embodiments, the kits provided herein employ means for detecting the expression of a biomarker by quantitative real-time PCR (qRT-PCR), microarray, flow cytometry or immunofluorescence. In other embodiments, the expression of the biomarker is measured by ELISA-based methodologies or other similar methods known in the art.

In another specific embodiment, the pharmaceutical or assay kit comprises, in a container, a compound or a pharmaceutical composition thereof, and further comprises, in one or more containers, components for isolating protein. In another specific embodiment, the pharmaceutical or assay kit comprises, in a container, a compound or a pharmaceutical composition, and further comprises, in one or more containers, components for conducting flow cytometry or an ELISA.

In another aspect, provided herein are kits for measuring biomarkers providing the materials necessary to measure the abundance of one or more of the gene products of the genes or a subset of genes (e.g., one, two, three, four, five or more genes) of the biomarkers provided herein. Such kits may comprise materials and reagents required for measuring RNA or protein. In some embodiments, such kits include microarrays, wherein the microarray is comprised of oligonucleotides and/or DNA and/or RNA fragments which hybridize to one or more of the products of one or more of the genes or a subset of genes of the biomarkers provided herein, or any combination thereof. In some embodiments, such kits may include primers for PCR of either the RNA product or the cDNA copy of the RNA product of the genes or subset of genes, or both. In some embodiments, such kits may include primers for PCR as well as probes for Quantitative PCR. In some embodiments, such kits may include multiple primers and multiple probes wherein some of said probes have different fluorophores so as to permit multiplexing of multiple products of a gene product or multiple gene products. In some embodiments, such kits may further include materials and reagents for creating cDNA from RNA. In some embodiments, such kits may include antibodies specific for the protein products of a gene or subset of genes of the biomarkers provided herein. Such kits may additionally comprise materials and reagents for isolating RNA and/or proteins from a biological sample. In addition such kits may include materials and reagents for synthesizing cDNA from RNA isolated from a biological sample. In some embodiments, such kits may include, a computer program product embedded on computer readable media for predicting whether a patient is clinically sensitive to a compound. In some embodiments, the kits may include a computer program product embedded on a computer readable media along with instructions.

In some embodiments, kits for measuring the expression of one or more nucleic acid sequences of a gene or a subset of genes of the biomarkers provided herein. In a specific embodiment, such kits measure the expression of one or more nucleic acid sequences associated with a gene or a subset of genes of the biomarkers provided herein. In accordance with this embodiment, the kits may comprise materials and reagents that are necessary for measuring the expression of particular nucleic acid sequence products of genes or a subset of genes of the biomarkers provided herein. For example, a microarray or RT-PCR kit may be produced for a specific condition and contain only those reagents and materials necessary for measuring the levels of specific RNA transcript products of the genes or a subset of genes of the biomarkers provided herein to predict whether a hematological cancer in a patient is clinically sensitive to a compound. Alternatively, in some embodiments, the kits can comprise materials and reagents that are not limited to those required to measure the expression of particular nucleic acid sequences of any particular gene of the biomarkers provided herein. For example, in certain embodiments, the kits comprise materials and reagents necessary for measuring the levels of expression of 1, 2, 3, 4, 5, 6, 7, 8, 9, 10, 15, 20, 25, 30, 35, 40, 45, 50 or more of the genes of the biomarkers provided herein, in addition to reagents and materials necessary for measuring the levels of the expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more genes other than those of the biomarkers provided herein. In other embodiments, the kits contain reagents and materials necessary for measuring the levels of expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more of the genes of the biomarkers provided herein, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are genes not of the biomarkers provided herein, or 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000 or 500-1000 genes that are genes not of the biomarkers provided herein.

For nucleic acid microarray kits, the kits generally comprise probes attached to a solid support surface. In one such embodiment, probes can be either oligonucleotides or longer length probes including probes ranging from 150 nucleotides in length to 800 nucleotides in length. The probes may be labeled with a detectable label. In a specific embodiment, the probes are specific for one or more of the gene products of the biomarkers provided herein. The microarray kits may comprise instructions for performing the assay and methods for interpreting and analyzing the data resulting from the performance of the assay. In a specific embodiment, the kits comprise instructions for predicting whether a hematological cancer in a patient is clinically sensitive to a compound. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own a suitable container.

In certain embodiments, a nucleic acid microarray kit comprises materials and reagents necessary for measuring the levels of expression of 1, 2, 3, 4, 5, 6, 7, 8, 9, 10, 15, 20, 25, 30, 35, 40, 45, 50 or more of the genes identified of the biomarkers provided herein, or a combination thereof, in addition to reagents and materials necessary for measuring the levels of the expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more genes other than those of the biomarkers provided herein. In other embodiments, a nucleic acid microarray kit contains reagents and materials necessary for measuring the levels of expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more of the genes of the biomarkers provided herein, or any combination thereof, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not of the biomarkers provided herein, or 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000 or 500-1000 genes that are not of the biomarkers provided herein.

For Quantitative PCR, the kits generally comprise pre-selected primers specific for particular nucleic acid sequences. The Quantitative PCR kits may also comprise enzymes suitable for amplifying nucleic acids (e.g., polymerases such as Taq), and deoxynucleotides and buffers needed for the reaction mixture for amplification. The Quantitative PCR kits may also comprise probes specific for the nucleic acid sequences associated with or indicative of a condition. The probes may or may not be labeled with a fluorophore. The probes may or may not be labeled with a quencher molecule. In some embodiments the Quantitative PCR kits also comprise components suitable for reverse-transcribing RNA including enzymes (e.g., reverse transcriptases such as AMV, MMLV and the like) and primers for reverse transcription along with deoxynucleotides and buffers needed for the reverse transcription reaction. Each component of the quantitative PCR kit is generally in its own suitable container. Thus, these kits generally comprise distinct containers suitable for each individual reagent, enzyme, primer and probe. Further, the quantitative PCR kits may comprise instructions for performing the assay and methods for interpreting and analyzing the data resulting from the performance of the assay. In a specific embodiment, the kits contain instructions for predicting whether a hematological cancer in a patient is clinically sensitive to a compound.

For antibody based kits, the kit can comprise, for example: (1) a first antibody (which may or may not be attached to a solid support) which binds to a peptide, polypeptide or protein of interest; and, optionally, (2) a second, different antibody which binds to either the peptide, polypeptide or protein, or the first antibody and is conjugated to a detectable label (e.g., a fluorescent label, radioactive isotope or enzyme). In a specific embodiment, the peptide, polypeptide or protein of interest is associated with or indicative of a condition (e.g., a disease). The antibody-based kits may also comprise beads for conducting an immunoprecipitation. Each component of the antibody-based kits is generally in its own suitable container. Thus, these kits generally comprise distinct containers suitable for each antibody. Further, the antibody-based kits may comprise instructions for performing the assay and methods for interpreting and analyzing the data resulting from the performance of the assay. In a specific embodiment, the kits contain instructions for predicting whether a hematological cancer in a patient is clinically sensitive to a compound.

In one embodiment a kit provided herein comprises a compound provided herein, or a pharmaceutically acceptable salt, solvate or hydrate thereof. Kits may further comprise additional active agents, including but not limited to those disclosed herein.

Kits provided herein may further comprise devices that are used to administer the active ingredients. Examples of such devices include, but are not limited to, syringes, drip bags, patches, and inhalers.

Kits may further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

In certain embodiments of the methods and kits provided herein, solid phase supports are used for purifying proteins, labeling samples or carrying out the solid phase assays. Examples of solid phases suitable for carrying out the methods disclosed herein include beads, particles, colloids, single surfaces, tubes, multiwell plates, microtiter plates, slides, membranes, gels and electrodes. When the solid phase is a particulate material (e.g., beads), it is, in one embodiment, distributed in the wells of multi-well plates to allow for parallel processing of the solid phase supports.

It is noted that any combination of the above-listed embodiments, for example, with respect to one or more reagents, such as, without limitation, nucleic acid primers, solid support and the like, are also contemplated in relation to any of the various methods and/or kits provided and the like, are also contemplated in relation to any of the various methods and/or kits provided herein.

Certain embodiments of the invention are illustrated by the following non-limiting examples.

### 6. EXAMPLES

The examples below are carried out using standard techniques, which are well known and routine to those of skill in the art, except where otherwise described in detail. The examples are intended to be merely illustrative.

### 6.1 Example 1

### Preparation of 3-(4-amino-1-oxo-1,3-dihydro-isoindol-2-yl)-piperidine-2,6-dione (lenalidomide)

### Methyl 2-bromomethyl-3-nitrobenzoate

A stirred mixture of methyl 2-methyl-3-nitrobenzoate (14.0 g, 71.7 mmol) and N-bromosuccinimide (15.3 g, 86.1 mmol) in carbon tetrachloride (200 mL) was heated under gentle reflux for 15 hours while a 100 W bulb situated 2 cm away was shining on the flask. The mixture was filtered and the solid was washed with methylene chloride (50 mL). The filtrate was washed with water (2×100 mL), brine (100 mL) and dried. The solvent was removed in vacuo and the residue was purified by flash chromatography (hexane/ethyl acetate, 8/2) to afford 19 g (96%) of the product as a yellow solid: mp 70.0-71.5° C.; 1H NMR (CDCl₃) δ 8.12-8.09 (dd, J=1.3 and 7.8 Hz, 1H), 7.97-7.94 (dd, J=1.3 and 8.2 Hz, 1H), 7.54 (t, J=8.0 Hz, 1H). 5.15 (s, 2H), 4.00 (s, 3H); ¹³C NMR (CDCl₃) δ 165.85, 150.58, 134.68, 132.38, 129.08, 127.80, 53.06, 22.69; HPLC, Water Nove-Pak/C18, 3.9×150 mm, 4 micron, 1 mL/min, 240 nm, 40/60 CH₃CN/0.1% H₃PO₄(aq) 7.27 min(98.92%); Anal. Calcd for C₉H₈NO₄Br:C, 39.44; H, 2.94; N, 5.11; Br, 29.15. Found: C, 39.46; H, 3.00; N, 5.00; Br, 29.11.

### t-Butyl N-(1-oxo-4-nitroisoindolin-2-yl)-L-glutamine

Triethylamine (2.9 g, 28.6 mmol) was added dropwise to a stirred mixture of methyl 2-bromomethyl-3-nitrobenzoate (3.5 g, 13.0 mmol) and L-glutamine t-butyl ester hydrochloride (3.1 g, 13.0 mmol) in tetrahydrofuran (90 mL). The mixture was heated to reflux for 24 hours. To the cooled mixture was added methylene chloride (150 mL) and the mixture was washed with water (2×40 mL), brine (40 mL) and dried. The solvent was removed in vacuo and the residue was purified by flash chromatography (3% CH₃OH in methylene chloride) to afford 2.84 g (60%) of crude product which was used directly in the next reaction: 1H NMR (CDCl₃) δ 8.40 (d, J=8.1 Hz, 1H), 8.15 (d, J=7.5 Hz, 1H), 7.71 (t, J=7.8 Hz, 1H), 5.83 (s, 1H), 5.61 (s, 1H), 5.12 (d, J=19.4 Hz, 1H), 5.04-4.98 (m, 1H), 4.92 (d, J=19.4 Hz, 1H), 2.49-2.22 (m, 4H). 1.46 (s, 9H); HPLC, Waters Nova-Pak C18, 3.9×150 mm, 4 micron, 1 mL/min, 240 nm, 25/75 CH₃CN/0.1% H₃PO₄(aq) 6.75 min(99.94%).

### N-(1-oxo-4-nitroisoindolin-2-yl)-L-glutamine

Hydrogen chloride gas was bubbled into a stirred 5° C. solution of t-butyl N-(1-oxo-4-nitro-isoindolin-2-yl)-L-glutamine (3.6 g, 9.9 mmol) in methylene chloride (60 mL) for 1 hour. The mixture was then stirred at room temperature for another hour. Ether (40 mL) was added and the resulting mixture was stirred for 30 minutes. The slurry was filtered, washed with ether and dried to afford 3.3 g of the product: 1H NMR (DMSO-d₆) δ 8.45 (d, J=8.1 Hz, 1H), 8.15 (d, J=7.5 Hz, 1H), 7.83 (t, J=7.9 Hz. 1H), 7.24 (s, 1H), 6.76 (s, 1H), 4.93 (s, 2H), 4.84-4.78 (dd, J=4.8 amd 10.4 Hz, 1H), 2.34-2.10 (m, 4H); ¹³C NMR (DMSO-d₆) δ 173.03, 171.88, 165.96, 143.35, 137.49, 134.77, 130.10, 129.61, 126.95, 53.65, 48.13, 31.50, 24.69; Anal. Calcd for C₁₃H₁₃N₃O₆: C, 50.82; H, 4.26; N, 13.68. Found: C, 50.53; H. 4.37; N, 13.22.

### (S)-3-(1-oxo-4-nitroisoindolin-2-yl)piperidine-2,6-dione

A stirred suspension mixture of N-(1-oxo-4-nitroisoindolin-2-yl)-L-glutamine (3.2 g, 10.5 mmol) in anhydrous methylene chloride (150 mL) was cooled to −40° C. with isopropanol/dry ice bath. Thionyl chloride (0.82 mL, 11.3 mmol) was added dropwise to the cooled mixture followed by pyridine (0.9 g. 11.3 mmol). After 30 min, triethylamine (1.2 g, 11.5 mmol) was added and the mixture was stirred at −30 to −40° C. for 3 hours. The mixture was poured into ice water (200 mL) and the aqueous layer was extracted with methylene chloride (40 mL). The methylene chloride solution was washed with water (2×60 mL), brine (60 mL) and dried. The solvent was removed in vacuo and the solid residue was slurried with ethyl acetate (20 mL) to give 2.2 g (75%) of the product as a white solid: mp 285° C.; 1H NMR (DMSO-d₆) δ: 1.04 (s, 1H), 8.49-8.45 (dd, J=0.8 and 8.2 Hz, 1H), 8.21-8.17 (dd, J=7.3 Hz, 1H), 7.84 (t, J=7.6 Hz, 1H), 5.23-5.15 (dd, J=4.9 and 13.0 Hz, 1H), 4.96 (dd, J=19.3 and 32.4 Hz, 2H), 3.00-2.85 (m, 1H), 2.64-2.49 (m, 2H), 2.08-1.98 (m, 1H); ¹³C NMR (DMSO-d₆) δ 172.79, 170.69, 165.93, 143.33, 137.40, 134.68, 130.15, 129.60, 127.02, 51.82, 48.43, 31.16. 22.23; HPLC, Waters Nove-Pak/C18, 3.9×150 mm, 4 micron, 1 mL/min, 240 nm, 20/80 CH₃CN/0.1% H₃PO₄(aq) 3.67 min(100%); Anal. Calcd for C₁₃HₙN₃O₅: C, 53.98; H, 3.83; N, 14.53. Found: C, 53.92; H, 3.70; N, 14.10.

### 3-(4-amino-1-oxo-1,3-dihydro-isoindol-2-yl)-piperidine-2,6-dione

A mixture of (S)-3-(1-oxo-4-nitroisoindolin-2-yl)piperidine-2,6-dione (1.0 g, 3.5 mmol) and 10% Pd/C (0.3 g) in methanol (600 mL) was hydrogenated in a Parr-Shaker apparatus at 50 psi of hydrogen for 5 hours. The mixture was filtered through Celite and the filtrate was concentrated in vacuo. The solid was slurried in hot ethyl acetate for 30 min, filtered and dried to afford 0.46 g (51%) of the product as a white solid: mp 235.5-239° C.; ¹H NMR (DMSO-d₆) δ 11.01 (s, 1H). 7.19 (t, J=7.6 Hz, 1H). 6.90 (d. J=7.3 Hz, 1H), 6.78 (d, J=7.8 Hz, 1H), 5.42 (s, 2H). 5.12 (dd. J=5.1 and 13.1 Hz, 1H), 4.17 (dd, J=17.0 and 28.8 Hz, 2H), 2.92-2.85 (m, 1H). 2.64-2.49 (m, 1H). 2.34-2.27 (m, 1H), 2.06-1.99 (m, 1H); ¹³C NMR (DMSO-d₆) δ 172.85, 171.19, 168.84, 143.58, 132.22. 128.79, 125.56, 116.37, 110.39, 51.48, 45.49, 31.20, 22.74; HPLC. Waters Nova-Pak/C18, 3.9×150 mm, 4 micron, 1 mL/min, 240 nm, 10/90 CH₃CN/0.1% H₃PO₄(aq) 0.96 min(100%); Chiral analysis, Daicel Chiral Pak AD, 40/60 Hexane/IPA, 6.60 min(99.42%); Anal. Calcd for C₁₃H₁₃N₃O₃: C, 60.23; H, 5.05; N, 16.21. Found: C, 59.96; H. 4.98; N, 15.84.

3-(4-Amino-1-oxo-1,3-dihydro-isoindol-2-yl)-piperidine-2,6-dione may also be prepared by methods known in the art, for example, as provided in Treatment compounds of the Future, 2003, 28(5): 425-431, the entirety of which is incorporated by reference.

### 6.2 Preparation of 3-(5-amino-2-methyl-4-oxo-4H-quinazolin-3-yl)-piperidine-2,6-dione (Compound A)

To a solution of potassium hydroxide (16.1 g, 286 mmol) in water (500 mL), was added 3-nitrophthalimide (25.0 g, 130 mmol) in portion at 0° C. The suspension was stirred at 0° C. for 3 hrs, and then heated to 30° C. for 3 hrs. To the solution, was added HCl (100 mL, 6N). The resulting suspension was cooled to 0° C. for 1 hr. The suspension was filtered and washed with cold water (2×10 mL) to give 3-nitro-phthalamic acid as a white solid (24.6 g, 90% yield): ¹H NMR (DMSO-d₆) δ 7.69 (brs, 1H, NHH), 7.74 (t, J=8 Hz, 1H, Ar), 7.92 (dd, J=1, 8 Hz, 1H, Ar), 8.13 (dd, J=1, 8 Hz, 1H, Ar), 8.15 (brs, 1H, NHH), 13.59 (s, 1H, OH); ¹³C NMR (DMSO-d₆) δ 125.33, 129.15, 130.25, 132.54, 136.72, 147.03, 165.90, 167.31.

To a mixture of 3-nitro-phthalamic acid (24.6 g, 117 mmol) and potassium hydroxide (6.56 g, 117 mmol) in water (118 mL), was added a mixture of bromine (6 mL), potassium hydroxide (13.2 g, 234 mmol) in water (240 mL) at 0° C., followed by addition of a solution of potassium hydroxide (19.8 g, 351 mmol) in water (350 mL). After 5 minutes at 0° C., the mixture was heated in a 100° C. oil bath for 1 hr. The reaction solution was cooled to room temperature, and then, in an ice-water bath for 30 minutes. To the mixture, a solution of HCl (240 mL, 2N) was added dropwise at 0° C., and the resulting mixture was kept for 1 hr. The suspension was filtered and washed with water (5 mL) to give 2-amino-6-nitro-benzoic acid as yellow solid (15.6 g, 73% yield): HPLC: Waters Symmetry C₁₈, 5 μm, 3.9×150 mm, 1 mL/min, 240 nm, CH₃CN/0.1% H₃PO₄, 5% grad to 95% over 5 min, 5.83 min (85%); ¹H NMR (DMSO-d₆) δ 6.90 (dd, J=1, 8 Hz, 1H, Ar), 7.01 (dd, J=1, 9 Hz, 1H, Ar), 7.31 (t, J=8 Hz, 1H, Ar), 8.5-9.5 (brs, 3H, OH, NH₂); ¹³C NMR (DMSO-d₆) δ 105.58, 110.14, 120.07, 131.74, 149.80, 151.36, 166.30; LCMS: MH=183.

A mixture of 2-amino-6-nitro-benzoic acid (1.5 g, 8.2 mmol) in acetic anhydride (15 mL) was heated at 200° C. for 30 minutes in a microwave oven. The mixture was filtered and washed with ethyl acetate (20 mL). The filtrate was concentrated in vacuo. The solid was stirred in ether (20 mL) for 2 hrs. The suspension was filtered and washed with ether (20 mL) to give 2-methyl-5-nitro-benzo[d][1,3]oxazin-4-one as a light brown solid (1.4 g, 85% yield): HPLC: Waters Symmetry C_{B}, 5 μm, 3.9×150 mm, 1 mL/min, 240 nm, CH₃CN/0.1% H₃PO₄, 5% grad 95% in 5 min, 5.36 min (92%); ¹H NMR (DMSO-d₆) δ 2.42 (s, 3H, CH₃), 7.79 (dd, J=1, 8 Hz, 1H, Ar), 7.93 (dd, J=1, 8 Hz, 1H, Ar), 8.06 (t, J=8 Hz, 1H, Ar); ¹³C NMR (DMSO-d₆) δ 20.87, 107.79, 121.54, 128.87, 137.19, 147.12, 148.46, 155.18, 161.78; LCMS: MH=207.

Two vials each with a suspension of 5-nitro-2-methyl-benzo[d][1,3]oxazin-4-one (0.60 g, 2.91 mmol) and 3-amino-piperidine-2,6-dione hydrogen chloride (0.48 g, 2.91 mmol) in pyridine (15 mL) were heated at 170° C. for 10 minutes in a microwave oven. The suspension was filtered and washed with pyridine (5 mL). The filtrate was concentrated in vacuo. The resulting mixture was stirred in HCl (30 mL, 1N), ethyl acetate (15 mL) and ether (15 mL) for 2 hrs. The suspension was filtered and washed with water (30 mL) and ethyl acetate (30 mL) to give a dark brown solid, which was stirred with methanol (50 mL) at room temperature overnight. The suspension was filtered and washed with methanol to give 3-(2-methyl-5-nitro-4-oxo-4H-quinazolin-3-yl)-piperidine-2,6-dione as a black solid (490 mg, 27% yield). The solid was used in the next step without further purification.

A mixture of 3-(2-methyl-5-nitro-4-oxo-4H-quinazolin-3-yl)-piperidine-2,6-dione (250 mg) and Pd(OH)₂ on carbon (110 mg) in DMF (40 mL) was shaken under hydrogen (50 psi) for 12 hrs. The suspension was filtered through a pad of Celite and washed with DNIF (10 mL). The filtrate was concentrated in vacuo and the resulting oil was purified by flash column chromatography (silica gel, methanol/methylene chloride) to give 3-(5-amino-2-methyl-4-oxo-4H-quinazolin-3-yl)-piperidine-2,6-dione as a white solid (156 mg, 69% yield): HPLC: Waters Symmetry C₁₈, 5 μm, 3.9×150 mm, 1 mL/min, 240 nm, 10/90 CH₃CN/0.1% H₃PO₄, 3.52 min (99.9%); mp: 293-295° C.; ¹H NMR (DMSO-d₆) δ 2.10-2.17 (m, 1H, CHH), 2.53 (s, 3H, CH₃), 2.59-2.69 (m, 2H, CH₂), 2.76-2.89 (m, 1H, CHH), 5.14 (dd, J=6, 11 Hz, 1H, NCH), 6.56 (d, J=8 Hz, 1H, Ar), 6.59 (d, J=8 Hz, 1H, Ar), 7.02 (s, 2H, NH₂), 7.36 (t, J=8 Hz, 1H, Ar), 10.98 (s, 1H, NH); ¹³C NMR (DMSO-d₆) δ 20.98, 23.14, 30.52, 55.92, 104.15, 110.48, 111.37, 134.92, 148.17, 150.55, 153.62, 162.59, 169.65, 172.57; LCMS: MH=287; Anal. Calcd. for C14H14N4O3+0.3 H2O: C, 57.65; H, 5.05; N, 19.21. Found: C, 57.50; H, 4.73; N, 19.00.

### 6.3 Example 3

### Analysis of Effects of Compound A and Lenalidomide on Primary CLL Samples by TMT-Tagging and Mass-Spectrometry

This example describes comparative and functional analysis of mass-spectrometry (MS) profiling data from primary CLL patients exposed to lenalidomide or Compound A.

### Methods

Data import: Relative abundance (RA) data was imported into the R statistical programming environment v.3.1.2. Proteins with less than one spectral count in any of the three MS runs for each case scenario were filtered out and relative abundance was scale normalized to the same median by using normalizeBetweenArrays( ) function implemented in the limma package for Bioconductor (see Yang et al., Nucleic Acids Research, 30(4), e15 (2002)).

Comparative analysis: RA scores were log 2-transformed to perform direct comparison of compounds exposure versus DMSO control and a paired moderated t-test (see Smyth, Statistical Applications in Genetics and Molecular Biology, 3(1), article 3 (2004)) was performed by using lmfit( ) and eBayes( ) functions form limma package to test differential relative abundance between experimental conditions. For differential analysis across cases, the log-2 fold changes of replicates for two of the case scenarios were considered. Significance p-values obtained from the analysis were corrected for multiple testing by the false-discovery BH method (see Benjamini and Hochberg, Journal of the Royal Statistical Society, Series B (Methodological), 57(1): 289-300 (1995)) and deemed significant at FDR threshold of 0.05 (5%).

Linear regression analysis: Linear regression was modeled by using lm( ) function from the stats package. This function provided the correlation coefficients as well as the linear fitted model. Data were assessed for outliers detection within the linear model via the Studentized residuals method (implemented in outlierTest( ) function from car package) and deemed significant if their q-values were under a threshold of 0.05 (5%).

Pathway enrichment analysis: Gene Set Enrichment Analysis (GSEA) was applied to ranked relative abundance ratios derived from the differential analysis for each condition and case scenario. Weighted statistics were used instead of classic unweighted enrichment calculations to take into account fold change differences rather than protein ranking. Gene categories assessed for enrichment correspond to the canonical pathways compendium (e.g., Reactome, Biocarta, KEGG) obtained from MSigDB (see Liberzon et al., Molecular signatures database (MSigDB) 3.0. Bioinformatics (Oxford, England), 27(12): 1739-40 (2011)). Enrichment p-values were corrected for multiple testing (using the method described in Storey and Tibshirani, Proceedings of the National Academy of Sciences, 100(16): 9440-9445 (2003)) and deemed significant at FDR threshold of 0.05 (5%).

### Results

### Differentially Affected Proteins by Lenalidomide and Compound A

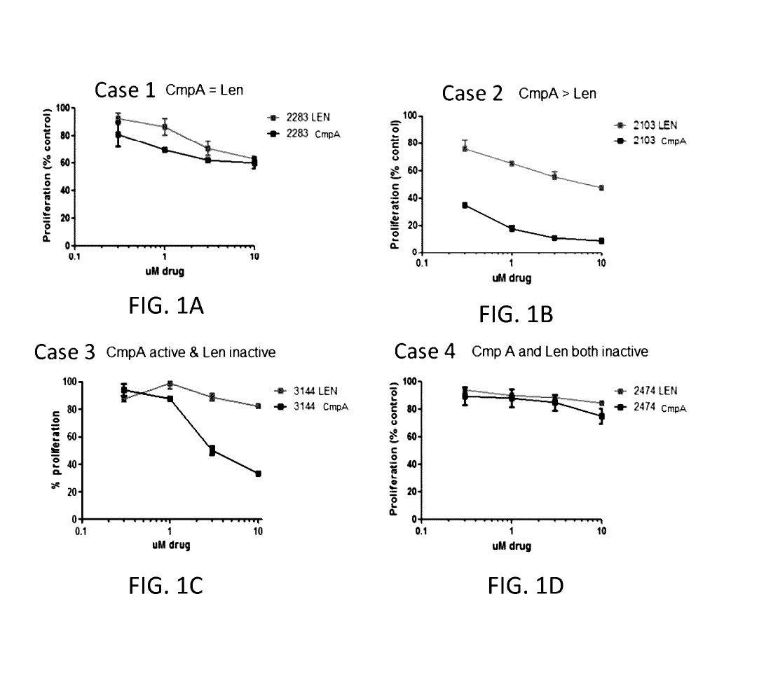

The samples used in this study were from four groups of CLL patients. Each group of CLL patients exhibited a different phenotype upon lenalidomide or Compound A treatment in in vitro proliferation assays. In the proliferation assays, lenalidomide and Compound A had similar effects on decreasing cell proliferation in the samples from the first group of patients (Case B1). Compound A had stronger proliferation reduction effect than lenalidomide on the samples from the second group of patients (Case B2). For the samples from the third group of patients (Case B3), Compound A reduced cell proliferation; while lenalidomide had no effect on cell proliferation. Finally, neither Compound A nor lenalidomide had any effect on cell proliferation for samples from the fourth group of patients (Case B4). The effects of Compound A or lenalidomide on these four groups of patients are shown in FIGS. 1A-1D.

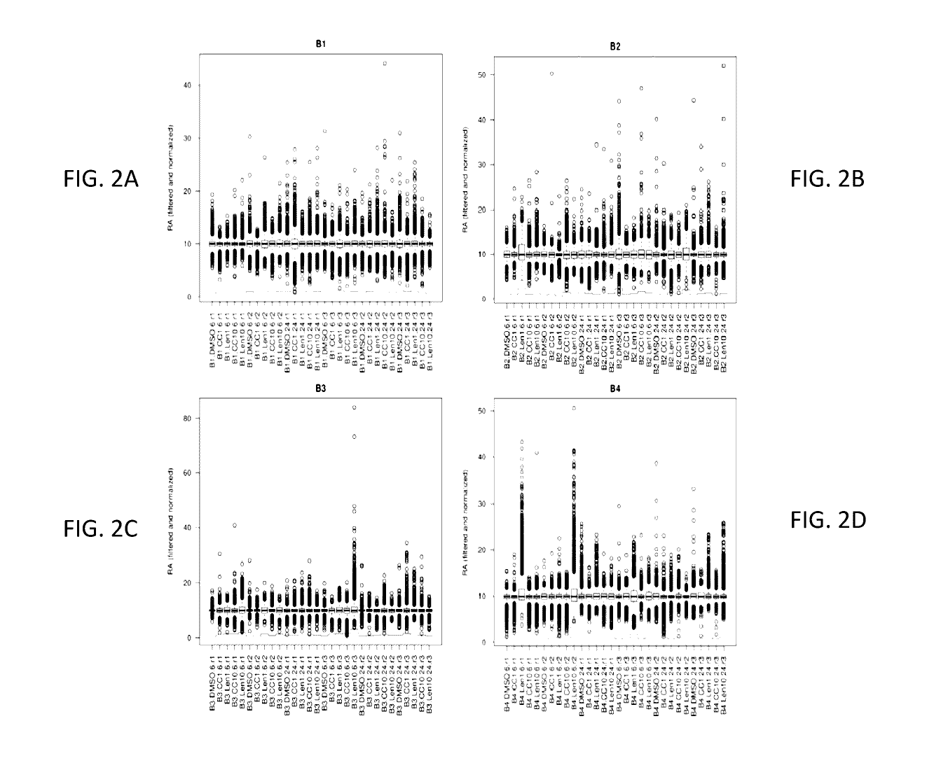

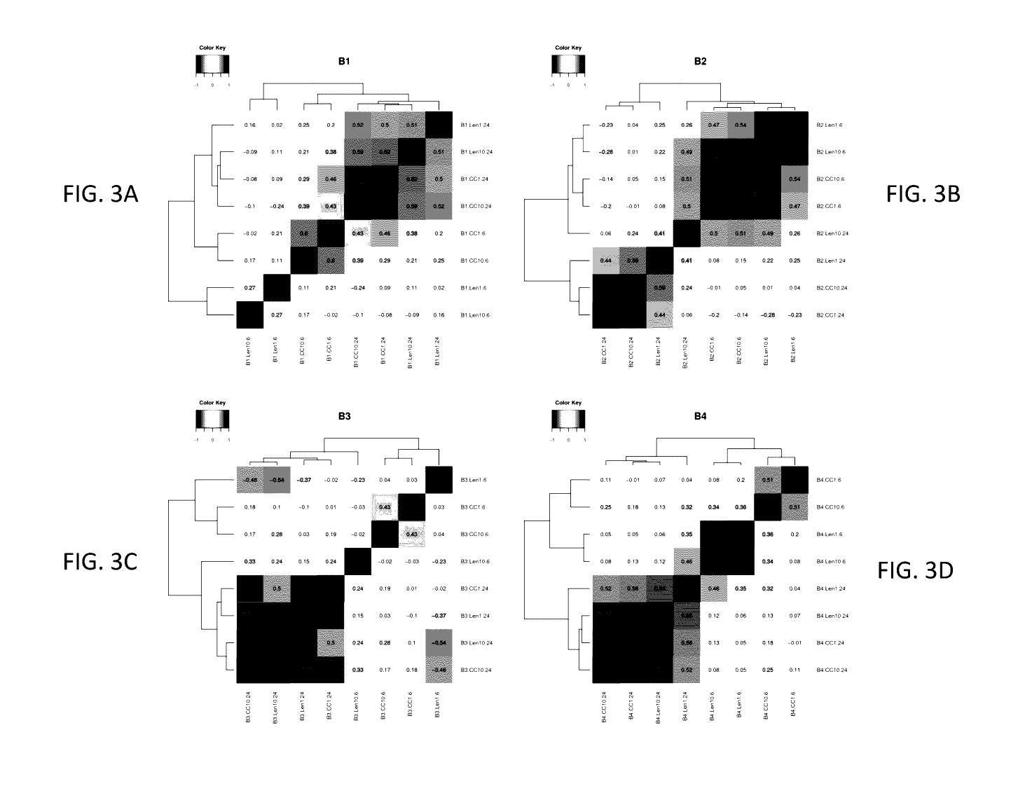

The samples from the four groups of patients (Case B1-B4) were treated with various amount of Compound A (e.g., 6 μM or 10 lenalidomide (e.g., 6 μM or 10 or DMSO (control) for various time period (e.g., 6 h or 24 h). Protein profiles (protein abundances) for the samples post treatment were studied using TMT-tagging and Mass-spectrometry (MS). Across case scenarios, each experimental condition was performed in triplicate with protein relative abundances assayed by 10-plex TMT isotopic labeling in three separate MS runs. MS quantified peptides were assigned to proteins by mapping to a human protein database, using Uniprot and gene-symbol identifiers with some proteins represented by more than one isoform. The proteins subjected to the analysis were those with more than one spectral count matched in three MS runs representing each case scenario. A total of 6114, 6070, 5982 and 5382 proteins were retained for each case scenario. Differential expression analysis was applied to the high and consistent coverage of 4594 proteins quantified across all case scenarios. FIGS. 2A-2D show distribution of normalized relative abundances for each replicate of the four case scenarios (Cases B1-B4). The standard deviation of each replicate is represented as a stair-plot line at the bottom of the boxplot. FIGS. 3A-3D show heatmaps representing correlations of log 2 fold-changes of protein levels post compound treatment as compared with DMSO across conditions for all case scenarios. Low correlation of log 2 fold-changes at 6 hours may be due to the alteration of a small subset of proteins at an early time point without strong general effects across the proteome. Stronger correlations at 24 hours can be linked to a higher proportion of proteins perturbed reflecting downstream changes upon compound exposure.

The effects of Compound A or lenalidomide (as compared with DMSO) on protein relative abundances were analyzed for each of the four cases of patient groups.

Furthermore, the effects of Compound A were also compared with those of lenalidomide for each case. Relative protein abundance changes at early times post Compound A or lenalidomide exposure reflected mainly the initial compound substrates (e.g., IKZF1, IKZF3 or PDE6D) with no significant changes across all case scenarios. Thus, comparisons across case scenarios and between compounds were performed at the higher concentration and longer time exposure, i.e., 10 μM of Compound A or lenalidomide for 24 hours exposure.

Direct comparison of protein relative abundances post lenalidomide treatment versus Compound A treatment (10 μM, 24 hours) identified a limited number of proteins which levels changed differently in response to Compound A treatment as compared to lenalidomide treatment for each case-21 significant proteins for Case B1, 2 significant proteins for Case B2, 15 significant proteins for Case B3, and 1 significant protein for Case B4 ((limma paired t-test FDR 5%). Tables 1-4 summarize proteins with significant changes in protein abundance in the lenalidomide versus Compound A comparison for each case scenario.

The proteins which levels were differentially affected by Compound A and lenalidomide in Case B1 are listed in Table 1 below, and include BCL2L1, GZMB, IGHM, IKZF1(isoform 1), IKZF1 (isoform 2), IRF8, KLF13, LGALS9, MARCKS, NDE1, NFKBIE, PTK2B, SAMSN1, SELL, SLAMF1, SNX20, SOD2, TCF7, TRAF1, WIZ, and ZBTB10. Among these proteins, BCL2L1, GZMB, IGHM, IRF8, KLF13, LGALS9, MARCKS, NDE1, NFKBIE, SNX20, TCF7, WIZ, and ZBTB10 were not differentially affected in other cases, and thus can be unique markers for CLL patients that are responsive to both Compound A and lenalidomide treatment. For example, the level of WIZ (widely-interspaced zinc-finger) decreased upon Compound A exposure and increased upon lenalidomide exposure.

The proteins which levels were differentially affected by Compound A and lenalidomide in Case B2 are listed in Table 2 below, and include IKZF1 and IKZF3.

The proteins which levels were differentially affected by Compound A and lenalidomide in Case B3 are listed in Table 3 below, and include CD40, CR2, CTSS, GBP1, IKZF1, IKZF3, ISG20, PTK2B, SAMSN1, SASH1, SELL, SEMA7A, SLAMF1, SOD2, and TRAF1. Among these proteins, CD40, CR2, CTSS, GBP1, ISG20, SASH1, and SEMA7A were not differentially affected in other cases, and thus can be unique markers for CLL patients who are only responsive Compound A, but not to lenalidomide treatment.

Because Cases B2 and B3 represent patients who respond to Compound A better than lenalidomide, differentially affected proteins identified only in B2 and B3 can be used to select patients responsive to Compound A, and these proteins include IKZF3, CD40, CR2, CTSS, GBP1, ISG20, SASH1, and SEMA7A.

The protein which level was differentially affected by Compound A and lenalidomide in Case B4 is listed in Table 4 below, and it is PDE6D. PDE6D was not identified as differentially affected protein in other cases, and thus can be used as a marker for patient not responsive to both lenalidomide and compound A.

It is clear that the set of differentially impacted proteins is different in each case. Thus, the level changes of these proteins can be used to identify patients sensitive to lenalidomide or Compound A treatment, as indicated above. In addition, since only patients of Case B4 do not respond to Compound A, the proteins differentially affected in Cases B1-B3 (shown in Tables 1-3) can be used as biomarkers for identifying patients responsive to Compound A treatment.

The differentially affected proteins by lenalidomide and Compound A include three key proteins directly or indirectly involved in leukocyte trafficking during inflammation, and they are SELL, SNX20, and KLF13. These proteins were specifically altered (down-regulated) upon Compound A exposure, but were not affected or slightly increased in protein abundance levels upon lenalidomide exposure.

SELL (or CD26L) is a protein directly involved in leukocytes and endothelial cells migration into secondary lymphoid organs and inflammation sites. SELL has been reported as a therapeutic target that promotes apoptosis of CLL cells, but not PBMCs from healthy donors. See Burgess et al., Clinical Cancer Research, 19: 5675 (2013).

SNX20 (Selecting Ligand Integrator Cytoplasmatic-1, SLIC1) is a sorting protein that specifically interacts with SELPLG, a glycoprotein that plays a key role in leukocyte transport during inflammation and functions as a high-affinity receptor on myeloid cells and T lymphocytes. See Schaff et al., European Journal of Immunology, 38(2): 550-564 (2008).

KLF13 is a transcription factor involved in hematopoietic development, see Outram et al., Cell Cycle, 7(13): 2047-2055 (2008), and is required for expression of several oncogenes, see Henson and Gollin, Cytogenetic and Genome Research, 128(4): 192-198 (2010). This protein regulates the expression of chemokine CCL5, which plays a key role in recruiting leukocytes to inflammatory sites. See Kim et al., Blood, 120(8): 1658-1667 (2012).

TABLE 1
Proteins with significant changes in protein abundance in response to
lenalidomide treatment as compared with Compound A treatment for Case B1

| | | | | | | | RA upon |
|---|---|---|---|---|---|---|---|
| | | | | | | | Cmp A vs. |
| ProteinID | symbol | logFC | AveExpr | t | P.Value | adj.P.Val | len treatment |
| Q07817 | BCL2L1 | 0.5325771 | 3.416067 | 9.069825 | 1.49E−004 | 0.049091054 | Higher |
| P10144 | GZMB | −1.1754786 | 3.267313 | −18.242791 | 3.36E−006 | 0.005135983 | Lower |
| P01871-2 | IGHM | −0.6625452 | 3.015658 | −12.211818 | 3.02E−005 | 0.015673133 | Lower |
| Q13422-2 | IKZF1 | −2.1691201 | 2.490719 | −13.781454 | 1.57E−005 | 0.015673133 | Lower |
| Q13422 | IKZF1 | −1.7088783 | 2.396327 | −12.899802 | 2.25E−005 | 0.015673133 | Lower |
| Q02556 | IRF8 | −0.5643035 | 3.146473 | −8.940988 | 1.61E−004 | 0.049091054 | Lower |
| Q9Y2Y9 | KLF13 | −1.0918841 | 3.131575 | −20.170189 | 1.93E−006 | 0.003932237 | Lower |
| O00182 | LGALS9 | −0.5645583 | 3.28323 | −10.46099 | 6.96E−005 | 0.032756131 | Lower |
| P29966 | MARCKS | 1.4381989 | 3.508117 | 28.228515 | 2.99E−007 | 0.00091374 | Higher |
| Q9NXR1 | NDE1 | 0.7281205 | 3.414287 | 12.316193 | 2.89E−005 | 0.015673133 | Higher |
| O00221 | NFKBIE | −0.7408206 | 3.122267 | −12.173253 | 3.08E−005 | 0.015673133 | Lower |
| Q14289-2 | PTK2B | 0.9814642 | 3.452799 | 10.314571 | 7.51E−005 | 0.032801786 | Higher |
| Q9NSI8 | SAMSN1 | 0.6554379 | 3.394789 | 9.010728 | 1.54E−004 | 0.049091054 | Higher |
| P14151-2 | SELL | −1.8231927 | 2.955669 | −31.685996 | 1.57E−007 | 0.00091374 | Lower |
| Q13291 | SLAMF1 | 0.9250806 | 3.436205 | 12.720449 | 2.42E−005 | 0.015673133 | Higher |
| Q7Z614 | SNX20 | −0.561076 | 3.267173 | −9.036312 | 1.52E−004 | 0.049091054 | Lower |
| P04179 | SOD2 | 0.6080631 | 3.37647 | 9.000844 | 1.55E−004 | 0.049091054 | Higher |
| P36402-3 | TCF7 | −0.7071182 | 3.143318 | −12.971767 | 2.18E−005 | 0.015673133 | Lower |
| Q13077 | TRAF1 | 0.7680421 | 3.416509 | 8.835991 | 1.71E−004 | 0.049758955 | Higher |
| M0QXA7 | WIZ | −0.4839155 | 3.195446 | −8.963292 | 1.58E−004 | 0.049091054 | Lower |
| Q96DT7 | ZBTB10 | 0.9381875 | 3.354879 | 12.978294 | 2.17E−005 | 0.015673133 | Higher |

TABLE 2
Proteins with significant changes in protein abundance in response to
lenalidomide treatment as compared with Compound A treatment for Case B2

| | | | | | | | RA upon |
|---|---|---|---|---|---|---|---|
| | | | | | | | Cmp Avs. |
| | | | | | | | len |
| ProteinID | symbol | logFC | AveExpr | t | P.Value | adj.P.Val | Treatment |
| Q13422 | IKZF1 | −2.18596 | 2.36411008 | −16.4662657 | 8.1231E−06 | 0.02811479 | Lower |
| Q9UKT9 | IKZF3 | −2.62559781 | 1.81313044 | −16.0629588 | 9.2635E−06 | 0.02811479 | Lower |

TABLE 3
Proteins with significant changes in protein abundance in response to
lenalidomide treatment as compared with Compound A treatment for Case B3

| | | | | | | | RA upon |
|---|---|---|---|---|---|---|---|
| | | | | | | | Cmp Avs. |
| | | | | | | | len |
| ProteinID | symbol | logFC | AveExpr | t | P.Value | adj.P.Val | Treatment |
| P25942 | CD40 | 0.80638571 | 3.44273704 | 11.6352747 | 4.0926E−05 | 0.03496795 | Higher |
| P20023-3 | CR2 | 0.79254918 | 3.56130972 | 9.68194932 | 0.00010908 | 0.04523344 | Higher |
| P25774 | CTSS | 0.59319727 | 3.37568248 | 10.5042929 | 7.0741E−05 | 0.04203823 | Higher |
| P32455 | GBP1 | 1.11744676 | 3.6467316 | 15.1579498 | 9.7769E−06 | 0.02131678 | Higher |
| Q13422 | IKZF1 | −2.68582587 | 1.85083241 | −38.1912107 | 6.0101E−08 | 0.00035947 | Lower |
| Q9UKT9 | IKZF3 | −1.42537816 | 1.93568979 | −10.0109233 | 9.1372E−05 | 0.04203823 | Lower |
| Q96AZ6 | ISG20 | 0.62344514 | 3.54462357 | 9.6103587 | 0.00011344 | 0.04523344 | Higher |
| Q14289-2 | PTK2B | 0.83613378 | 3.49935787 | 12.9039686 | 2.3429E−05 | 0.02802518 | Higher |
| Q9NSI8 | SAMSN1 | 0.89001348 | 3.61314753 | 13.5364277 | 1.8082E−05 | 0.02703725 | Higher |
| O94885 | SASH1 | 0.71773707 | 3.56842397 | 10.2580066 | 8.0268E−05 | 0.04203823 | Higher |
| P14151-2 | SELL | −1.02955479 | 3.23871719 | −10.2261784 | 8.1606E−05 | 0.04203823 | Lower |
| O75326 | SEMA7A | 0.90396484 | 3.52909851 | 10.6111982 | 6.7022E−05 | 0.04203823 | Higher |
| Q13291 | SLAMF1 | 1.08621186 | 3.68367114 | 14.910901 | 1.0692E−05 | 0.02131678 | Higher |
| P04179 | SOD2 | 0.63194818 | 3.47603603 | 10.0672577 | 8.869E−05 | 0.04203823 | Higher |
| Q13077 | TRAF1 | 0.7522986 | 3.51959894 | 12.1078132 | 3.3037E−05 | 0.03293204 | Higher |

TABLE 4
Proteins with significant changes in protein abundance in response to
lenalidomide treatment as compared with Compound A treatment for Case B4

| | | | | | | | RA upon |
|---|---|---|---|---|---|---|---|
| | | | | | | | Cmp Avs. |
| | | | | | | | len |
| ProteinID | symbol | logFC | AveExpr | t | P.Value | adj.P.Val | Treatment |
| O43924 | PDE6D | −1.49421612 | 2.9563014 | −20.5121834 | 4.9885E−06 | 0.02684289 | Lower |

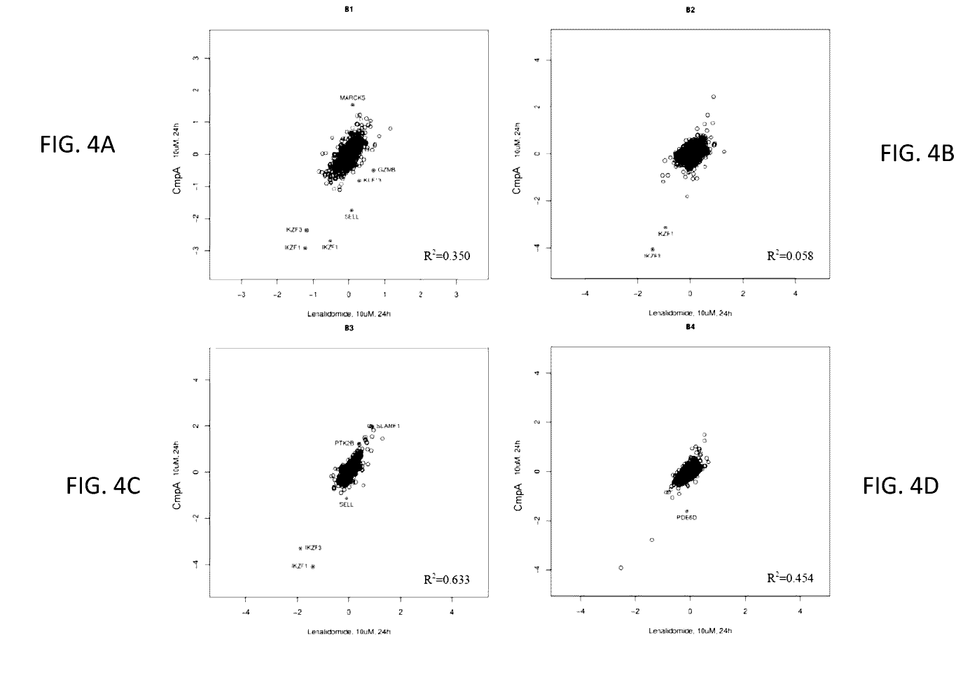

Log 2 ratios of relative proteins abundances for lenalidomide and Compound A exposed samples (10 μM, 24 hours) against corresponding DMSO controls (averaged across replicates) are plotted for each case scenario in FIGS. 4A-4D. Representative proteins differentially affected by lenalidomide versus Compound A are identified in the figure. Those proteins with no significant difference in the lenalidomide vs Compound A comparison are close to the dashed line through the origin with unitary slope.

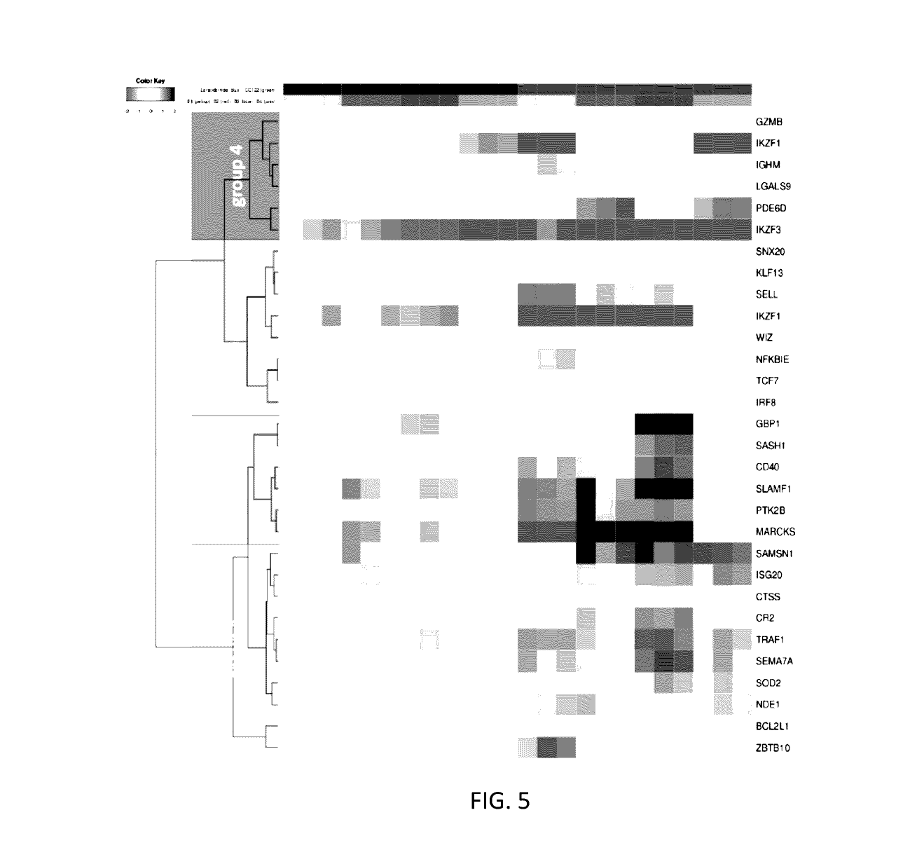

FIG. 5 shows a heatmap including proteins differentially affected by lenalidomide vs Compound A (10 μM, 24 hours). Color scale represents log 2 fold-change of relative protein abundance in compound as compared with DMSO (green and red shades denote decreased and increase abundance relative to DMSO control, respectively). Not quantified proteins in any of the experiments are depicted in grey. The blue color above the main heatmap represents samples treated with lenalidomide; the green color above the main heatmap represents samples treated with Compound A. Cases B1-B4 are represented by yellow, red, blue and pink, respectively. Proteins at rows are hierarchically clustered using Pearson correlation distance.

Four main clusters are identified in FIG. 5. Group 1 and Group 2 comprise proteins related to immune response (according to GO enrichment analysis), where lenalidomide and Compound A have the same regulatory effects on these proteins (up or down regulation) with higher effects upon Compound A exposure. Group 3 includes a cluster of eight proteins—SMNX20, SELL, KLF13, WIZ, IKZF1, NFKBIE, TCF7 and IRF8—which protein abundance levels decrease upon Compound A exposure, and do not change or increase upon lenalidomide exposure. IKZF1 (Ikaros) protein level decreases across all experimental conditions with stronger effect upon Compound A exposure. Group 4 includes 6 proteins including IKZF3 and a second IKZF1 isoform, and the levels of these proteins decrease significantly upon Compound A exposure, however, lenalidomide exposure has mild effects on these proteins.

### Response to Compound A in Different Patient Groups

Furthermore, Compound A effects were compared across four cases. Specifically, Cases B1, B2 and B3 where the patients were shown to be responsive to treatment with Compound A were compared to Case B4 where the patients were shown to be not responsive to Compound A. Comparison of protein abundance changes (log 2 ratios of 10 μM Compound A versus DMSO control) yielded no significant proteins (limma moderated t-test FDR 5%) for B1 against B4 or B2 against B4. Three proteins were identified as differentially modulated for B3 as compared with B4, and they were GBP4, LGALS9 and IRF5.

To recover additional differences in protein changes that may be hidden by the extent of the general alteration in the different scenarios, analysis of residuals from the linear regressions of log 2 fold-changes across scenarios was performed. Significant proteins were identified as the outliers (FDR q-value 5%) within the residuals analysis (these proteins are shown in Table 5).

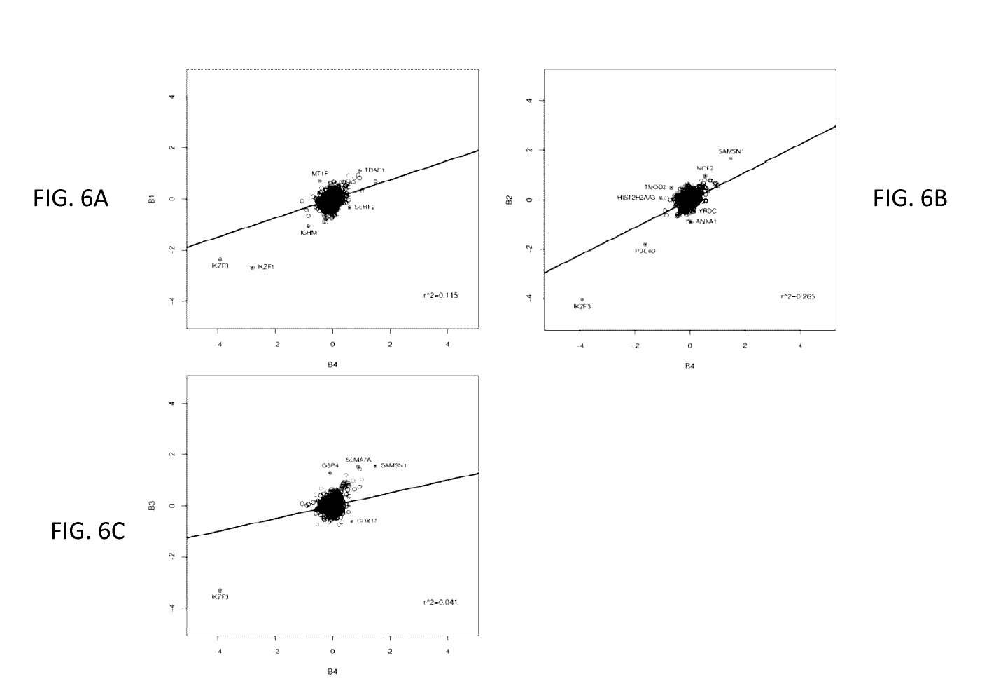

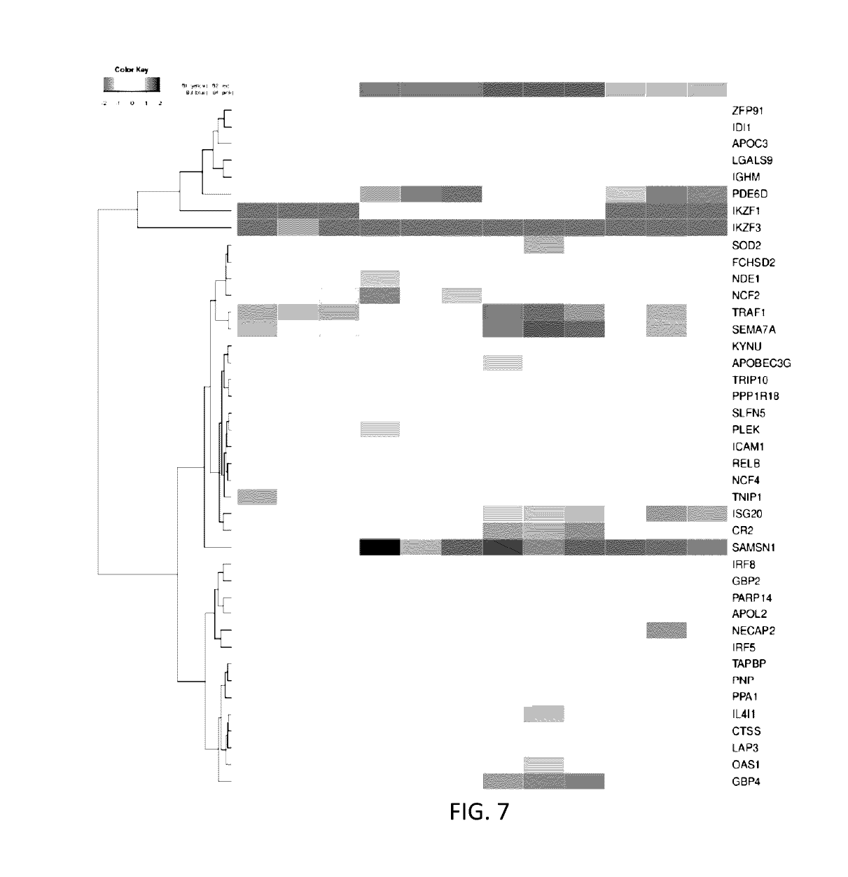

FIGS. 6A-6C represent log 2 fold-changes of relative abundances upon 24 hour exposure to 10 μM Compound A versus DMSO, including the linear regression models that were analyzed further. FIGS. 6A-6C show scatter plots comparing 10 μM Compound A effects at 24 hours in three different case scenarios (B1, B2, and B3). The expected behavior when there is no difference among cases is represented as a dashed line through the origin with unitary slope. Representative significant proteins from residuals analysis (FDR q-value 5%) are indicated in the plot. Proteins with no significant changes in response to Compound A treatment as compared with DMSO control in any of the case scenarios were filtered. Proteins that were identified as differentially perturbed in B1, B2 and B3 as compared with B4 are shown in FIG. 7. Generally stronger effects were observed in the scenario B3, where patients are responsive to Compound A but not to lenalidomdie.

The proteins which levels were affected differently upon Compound A exposure in any one of B1, B2, and B3 as compared to B4 are listed in Table 5. These proteins can also be used to predict a patient's response to Compound A treatment.

GBP4, LGALS9 and IRF5 represent the most significant differences between scenarios B3 and B4. Increase in GBP4 protein level occurs exclusively in B3. LGALS9 and IRF5 present a decrease in protein abundance in B4, but are not significantly altered in the other scenarios. These proteins may constitute potential early post-treatment response biomarkers. Beside these proteins, GBP2 (detected by regression analysis) shows a similar profile to GBP4 with increased abundance level only in B3.

This study shows that less interferon and inflammatory cytokine are induced by Compound A in less sensitive patient samples. GBP protein family members GBP2 and GBP4 are interferon-induced proteins with increased abundance levels upon Compound A exposure in the case scenario wherein the patients are responsive selectively to Compound A (Case B3). GBP2 provides a broad host protection against different pathogen classes and high level of this protein-encoding gene has been associated with better prognosis in breast cancer. See Godoy et al., Breast Cancer, 21(4): 491-499 (2014). GBP4 plays an important role in erythroid differentiation and its over-expression inhibits virus-triggered activation of IRF7-signaling. See Hu et al., Journal of Immunology, 187(12): 6456-62 (2011). IRF5, which protein level exclusively decreased in the scenario wherein the patients do not respond to both Compound A and lenalidomide (Case B4) (not quantified in B2), is involved in the induction of other interferons and inflammatory cytokines. P68 mutation of the IRF5 protein-coding gene results in complete loss of DNA binding and has been consistently found in CLL patients. See Yang et al., PLoS One, 4(5):e5500 (2009). Further characterization of mutations on this gene in the resistant patient samples may provide an avenue for the identification of putative resistance biomarkers.

TABLE 5
The proteins which levels are affected differently upon Compound A
exposure in any one of B1, B2, and B3 as compared to B4

| Symbol | Protein ID |
|---|---|
| APOBEC3G | Q9HC16 |
| APOC3 | P02656 |
| APOL2 | Q9BQE5 |
| CR2 | P20023-3 |
| CTSS | P25774 |
| FCHSD2 | O94868 |
| GBP2 | P32456 |
| GBP4 | Q96PP9 |
| ICAM1 | P05362 |
| IDI1 | Q13907-2 |
| IGHM | P01871-2 |
| IKZF1 | Q13422-2 |
| IKZF3 | Q9UKT9 |
| IL4I1 | Q96RQ9-2 |
| IRF5 | Q13568-2 |
| IRF8 | Q02556 |
| ISG20 | Q96AZ6 |
| KYNU | Q16719 |
| LAP3 | P28838 |
| LGALS9 | O00182 |
| NCF2 | P19878 |
| NCF4 | Q15080 |
| NDE1 | Q9NXR1 |
| NECAP2 | Q9NVZ3 |
| OAS1 | P00973 |
| PARP14 | Q460N5 |
| PDE6D | O43924 |
| PLEK | P08567 |
| PNP | P00491 |
| PPA1 | Q15181 |
| PPP1R18 | Q6NYC8 |
| RELB | Q01201 |
| SAMSN1 | Q9NSI8 |
| SEMA7A | O75326 |
| SLFN5 | Q08AF3 |
| SOD2 | P04179 |
| TAPBP | O15533-3 |
| TNIP1 | Q15025 |
| TRAF1 | Q13077 |
| TRIP10 | Q15642 |
| ZFP91 | Q96JP5 |

Similar analysis can also be applied to compare lenalidomide effects across cases. For example, GZMB protein expression level increased upon lenalidomide exposure in Case B1 (where the patients respond to lenalidomide treatment) and decreased in Case B4 (where the patients do not respond to lenalidomide treatment), suggesting an association of this protein with lenalidomide resistance mechanisms. This protein has a crucial role in cell-mediated immune response by rapid induction of target cell apoptosis by Cytotoxic T Lymphocytes (CTL). Benign human B-cells have been shown to produce high levels of GZMB in response to IL20. See Jahrsdörfer et al, Blood, 108(8): 2712-2719 (2006).

### Pathways Affected by Compound Treatment

GSEA described in Subramanian et al., Molecular signatures database (MSigDB) 3.0. Bioinformatics (Oxford, England), 27(12), 1739-40 (2011) was applied to identified biological processes that were altered upon compound exposure in all cases. Tables 6-9 show pathways significantly (FDR 5%) associated with an increase in protein abundance levels upon lenalidomide or Compound A exposure for any experimental condition when compared to DMSO controls.

TABLE 6
Pathways significantly (FDR 5%) associated with increase in protein abundance
levels upon Compound A or lenalidomide exposure in Case B1

| Condition | Name | Size | NOM.p.val | FDR.q.val | FWER.p.val |
|---|---|---|---|---|---|
| CmpA_10uM_24h | REACTOME_CELL_CELL_COMMUNICATION | 44 | 0 | 0.0050571547 | 0.008 |
| CmpA_10uM_24h | KEGG_LEUKOCYTE_TRANSENDOTHELIAL_MIGRATION | 59 | 0 | 0.006314017 | 0.005 |
| CmpA_10uM_24h | PID_FAK_PATHWAY | 49 | 0 | 0.013482939 | 0.052 |
| CmpA_10uM_24h | KEGG_FOCAL_ADHESION | 104 | 0 | 0.015889907 | 0.049 |
| CmpA_10uM_24h | PID_MET_PATHWAY | 63 | 0 | 0.016946595 | 0.039 |
| CmpA_10uM_24h | BIOCARTA_PYK2_PATHWAY | 25 | 0.0019417476 | 0.021189218 | 0.095 |
| CmpA_10uM_24h | KEGG_TIGHT_JUNCTION | 60 | 0 | 0.03602398 | 0.178 |
| CmpA_10uM_24h | BIOCARTA_CXCR4_PATHWAY | 20 | 0.002020202 | 0.04064273 | 0.219 |
| CmpA_10uM_24h | REACTOME_CELL_JUNCTION_ORGANIZATION | 20 | 0 | 0.0444116 | 0.262 |
| CmpA_10uM_24h | PID_NEPHRIN_NEPH1_PATHWAY | 21 | 0 | 0.04740394 | 0.303 |

TABLE 7
Pathways significantly (FDR 5%) associated with increase in protein abundance
levels upon Compound A or lenalidomide exposure in Case B2

| Condition | Name | Size | NOM.p.val | FDR.q.val | FWER.p.val |
|---|---|---|---|---|---|
| len_10uM_24h | REACTOME_RESPIRATORY_ELECTRON_TRANSPORT_ATP_SYNTHESIS_BY_CHEMIOSMOTIC_{—} | 63 | 0 | 0 | 0 |
| | COUPLING_AND_HEAT_PRODUCTION_BY_UNCOUPLING_PROTEINS_{—} | | | | |
| len_10uM_24h | REACTOME_TCA_CYCLE_AND_RESPIRATORY_ELECTRON_TRANSPORT | 96 | 0 | 0 | 0 |
| len_10uM_24h | REACTOME_RESPIRATORY_ELECTRON_TRANSPORT | 53 | 0 | 0 | 0 |
| len_10uM_24h | REACTOME_INTERFERON_GAMMA_SIGNALING | 33 | 0 | 0 | 0 |
| len_10uM_24h | KEGG_SYSTEMIC_LUPUS_ERYTHEMATOSUS | 38 | 0 | 0 | 0 |
| len_10uM_24h | KEGG_ALLOGRAFT_REJECTION | 20 | 0 | 0 | 0 |
| len_10uM_24h | KEGG_TYPE_I_DIABETES_MELLITUS | 20 | 0 | 0 | 0 |
| len_10uM_24h | KEGG_AUTOIMMUNE_THYROID_DISEASE | 20 | 0 | 0 | 0 |
| len_10uM_24h | KEGG_GRAFT_VERSUS_HOST_DISEASE | 18 | 0 | 0 | 0 |
| len_10uM_24h | KEGG_VIRAL_MYOCARDITIS | 40 | 0 | 7.72E+01 | 0.001 |
| len_10uM_24h | REACTOME_MRNA_PROCESSING | 142 | 0 | 8.42E+00 | 0.001 |
| len_10uM_24h | KEGG_OXIDATIVE_PHOSPHORYLATION | 79 | 0 | 9.26E+01 | 0.001 |
| len_10uM_24h | KEGG_LEISHMANIA_INFECTION | 32 | 0 | 0.0001537252 | 0.003 |
| len_10uM_24h | KEGG_COMPLEMENT_AND_COAGULATION_CASCADES | 16 | 0 | 0.00016276784 | 0.003 |
| len_10uM_24h | REACTOME_INTERFERON_ALPHA_BETA_SIGNALING | 25 | 0 | 0.00017294084 | 0.003 |
| len_10uM_24h | KEGG_ANTIGEN_PROCESSING_AND_PRESENTATION | 45 | 0 | 0.00018447022 | 0.003 |
| len_10uM_24h | KEGG_PARKINSONS_DISEASE | 76 | 0 | 0.00019338347 | 0.004 |
| len_10uM_24h | REACTOME_PROCESSING_OF_CAPPED_INTRON_CONTAINING_PRE_MRNA | 129 | 0 | 0.00019764666 | 0.003 |
| len_10uM_24h | REACTOME_MRNA_SPLICING | 101 | 0 | 0.00021285027 | 0.003 |
| len_10uM_24h | KEGG_INTESTINAL_IMMUNE_NETWORK_FOR_IGA_PRODUCTION | 16 | 0 | 0.00031984365 | 0.007 |
| len_10uM_24h | KEGG_ASTHMA | 14 | 0 | 0.0007484972 | 0.017 |
| len_10uM_24h | REACTOME_INTERFERON_SIGNALING | 90 | 0 | 0.0012125012 | 0.029 |
| len_10uM_24h | KEGG_HEMATOPOIETIC_CELL_LINEAGE | 18 | 0 | 0.0013629834 | 0.034 |
| len_10uM_24h | REACTOME_AMYLOIDS | 16 | 0 | 0.0013837913 | 0.036 |
| len_10uM_24h | REACTOME_IMMUNOREGULATORY_INTERACTIONS_BETWEEN_A_LYMPHOID_AND_A_NON_LYMPHOID_CELL | 15 | 0 | 0.0018084843 | 0.049 |
| len_10uM_24h | REACTOME_APOPTOSIS_INDUCED_DNA_FRAGMENTATION | 11 | 0 | 0.0020545726 | 0.058 |
| len_10uM_24h | REACTOME_CITRIC_ACID_CYCLE_TCA_CYCLE | 19 | 0 | 0.0026566784 | 0.077 |
| len_10uM_24h | REACTOME_PHOSPHORYLATION_OF_CD3_AND_TCR_ZETA_CHAINS | 8 | 0 | 0.0030197778 | 0.091 |
| len_10uM_24h | KEGG_TRYPTOPHAN_METABOLISM | 20 | 0 | 0.0040300484 | 0.132 |
| len_10uM_24h | REACTOME_TRANSLOCATION_OF_ZAP_70_TO_IMMUNOLOGICAL_SYNAPSE | 7 | 0.0020491802 | 0.0041329567 | 0.131 |
| len_10uM_24h | KEGG_SPLICEOSOME | 119 | 0 | 0.004244089 | 0.13 |
| len_10uM_24h | REACTOME_ENDOSOMAL_VACUOLAR_PATHWAY | 7 | 0 | 0.004787783 | 0.157 |
| len_10uM_24h | REACTOME_MITOCHONDRIAL_FATTY_ACID_BETA_OXIDATION | 14 | 0.0018867925 | 0.0055323145 | 0.187 |
| len_10uM_24h | REACTOME_MEIOSIS | 33 | 0 | 0.0057216655 | 0.197 |
| len_10uM_24h | KEGG_ALZHEIMERS_DISEASE | 100 | 0 | 0.0077088824 | 0.264 |
| len_10uM_24h | REACTOME_MEIOTIC_RECOMBINATION | 18 | 0 | 0.008901454 | 0.301 |
| len_10uM_24h | REACTOME_PD1_SIGNALING | 8 | 0.0019157088 | 0.00920681 | 0.321 |
| len_10uM_24h | REACTOME_RNA_POL_I_TRANSCRIPTION | 27 | 0.0019011407 | 0.009405304 | 0.319 |
| len_10uM_24h | REACTOME_TRANSPORT_OF_MATURE_TRANSCRIPT_TO_CYTOPLASM | 50 | 0 | 0.009491289 | 0.34 |
| len_10uM_24h | REACTOME_DOUBLE_STRAND_BREAK_REPAIR | 16 | 0.004032258 | 0.009686916 | 0.352 |
| len_10uM_24h | KEGG_HUNTINGTONS_DISEASE | 112 | 0 | 0.010872776 | 0.395 |
| len_10uM_24h | REACTOME_ANTIGEN_PROCESSING_CROSS_PRESENTATION | 61 | 0 | 0.012631842 | 0.448 |
| len_10uM_24h | REACTOME_NEF_MEDIATED_DOWNREGULATION_OF_MHC_CLASS_I_COMPLEX_CELL_SURFACE_EXPRESSION | 8 | 0 | 0.015250105 | 0.519 |
| len_10uM_24h | KEGG_FATTY_ACID_METABOLISM | 32 | 0.0021097045 | 0.016199393 | 0.545 |
| len_10uM_24h | REACTOME_G_ALPHA_I_SIGNALLING_EVENTS | 20 | 0.0037313432 | 0.016247477 | 0.555 |
| len_10uM_24h | KEGG_VALINE_LEUCINE_AND_ISOLEUCINE_DEGRADATION | 41 | 0 | 0.01665359 | 0.564 |
| len_10uM_24h | BIOCARTA_BLYMPHOCYTE_PATHWAY | 7 | 0 | 0.017409407 | 0.593 |
| len_10uM_24h | REACTOME_ANTIGEN_PRESENTATION_FOLDING_ASSEMBLY_AND_PEPTIDE_LOADING_OF_CLASS_I_MHC | 19 | 0.0038610038 | 0.01747548 | 0.622 |
| len_10uM_24h | KEGG_NATURAL_KILLER_CELL_MEDIATED_CYTOTOXICITY | 55 | 0 | 0.017648207 | 0.613 |
| len_10uM_24h | BIOCARTA_HIVNEF_PATHWAY | 41 | 0 | 0.017692257 | 0.606 |
| len_10uM_24h | REACTOME_PYRUVATE_METABOLISM_AND_CITRIC_ACID_TCA_CYCLE | 37 | 0.00203666 | 0.017806377 | 0.622 |
| len_10uM_24h | KEGG_CITRATE_CYCLE_TCA_CYCLE | 27 | 0.0019230769 | 0.019952651 | 0.679 |
| len_10uM_24h | BIOCARTA_PML_PATHWAY | 9 | 0.003787879 | 0.020933747 | 0.707 |
| len_10uM_24h | REACTOME_ER_PHAGOSOME_PATHWAY | 54 | 0 | 0.021081334 | 0.7 |
| len_10uM_24h | REACTOME_SRP_DEPENDENT_COTRANSLATIONAL_PROTEIN_TARGETING_TO_MEMBRANE | 99 | 0 | 0.021654036 | 0.721 |
| len_10uM_24h | PID_ATM_PATHWAY | 22 | 0 | 0.02208832 | 0.728 |
| len_10uM_24h | REACTOME_FORMATION_OF_ATP_BY_CHEMIOSMOTIC_COUPLING | 10 | 0.00390625 | 0.023880757 | 0.77 |
| len_10uM_24h | BIOCARTA_FAS_PATHWAY | 23 | 0.002 | 0.024119863 | 0.769 |
| len_10uM_24h | BIOCARTA_CTL_PATHWAY | 5 | 0 | 0.024188325 | 0.778 |
| len_10uM_24h | REACTOME_APOPTOTIC_EXECUTION_PHASE | 36 | 0.0019417476 | 0.024302661 | 0.763 |
| len_10uM_24h | PID_CD8TCRPATHWAY | 31 | 0 | 0.024814455 | 0.797 |
| len_10uM_24h | REACTOME_TRNA_AMINOACYLATION | 41 | 0.0019455253 | 0.026581544 | 0.819 |
| len_10uM_24h | REACTOME_RNA_POL_I_RNA_POL_III_AND_MITOCHONDRIAL_TRANSCRIPTION | 45 | 0.005882353 | 0.028929025 | 0.847 |
| len_10uM_24h | REACTOME_PACKAGING_OF_TELOMERE_ENDS | 7 | 0.004192872 | 0.031024037 | 0.877 |
| len_10uM_24h | PID_IL12_2PATHWAY | 24 | 0.004158004 | 0.032278974 | 0.89 |
| len_10uM_24h | REACTOME_RNA_POL_I_PROMOTER_OPENING | 10 | 0.003937008 | 0.033148613 | 0.903 |
| len_10uM_24h | REACTOME_GENERATION_OF_SECOND_MESSENGER_MOLECULES | 15 | 0.012295082 | 0.038439807 | 0.934 |
| len_10uM_24h | KEGG_CELL_ADHESION_MOLECULES_CAMS | 38 | 0 | 0.03960564 | 0.944 |
| len_10uM_24h | REACTOME_ADP_SIGNALLING_THROUGH_P2RY12 | 8 | 0.0037105752 | 0.040948864 | 0.95 |
| len_10uM_24h | BIOCARTA_TCRA_PATHWAY | 5 | 0 | 0.04175098 | 0.952 |
| len_10uM_24h | REACTOME_INTEGRATION_OF_PROVIRUS | 7 | 0.0058027077 | 0.04247452 | 0.954 |
| len_10uM_24h | REACTOME_CD28_DEPENDENT_VAV1_PATHWAY | 7 | 0.017716536 | 0.043686416 | 0.96 |
| len_10uM_24h | REACTOME_G_PROTEIN_ACTIVATION | 9 | 0.007736944 | 0.04421655 | 0.96 |
| len_10uM_24h | REACTOME_BRANCHED_CHAIN_AMINO_ACID_CATABOLISM | 17 | 0.0037453184 | 0.048257753 | 0.977 |
| len_10uM_24h | REACTOME_GPCR_LIGAND_BINDING | 20 | 0.011131725 | 0.04827034 | 0.973 |
| len_10uM_24h | PID_BARD1PATHWAY | 17 | 0.01119403 | 0.04835061 | 0.973 |
| len_10uM_24h | BIOCARTA_ASBCELL_PATHWAY | 5 | 0.0020449897 | 0.04866482 | 0.976 |
| len_10uM_24h | REACTOME_MITOCHONDRIAL_TRNA_AMINOACYLATION | 20 | 0.00967118 | 0.049373593 | 0.98 |
| len_10uM_24h | KEGG_BETA_ALANINE_METABOLISM | 13 | 0.0037453184 | 0.049393807 | 0.979 |
| len_10uM_24h | REACTOME_APOPTOSIS | 103 | 0 | 0.04982539 | 0.983 |
| CmpA_10uM_24h | REACTOME_KINESINS | 18 | 0 | 0 | 0 |
| CmpA_10uM_24h | PID_PLK1_PATHWAY | 22 | 0 | 0.00059934414 | 0.002 |
| CmpA_10uM_24h | PID_AURORA_B_PATHWAY | 26 | 0 | 0.00060082646 | 0.001 |
| CmpA_10uM_24h | REACTOME_FACTORS_INVOLVED_IN_MEGAKARYOCYTE_DEVELOPMENT_AND_PLATELET_PRODUCTION | 70 | 0 | 0.0007991255 | 0.002 |
| CmpA_10uM_24h | REACTOME_PHOSPHORYLATION_OF_THE_APC_C | 11 | 0 | 0.014446337 | 0.059 |
| CmpA_10uM_24h | REACTOME_CYCLIN_A_B1_ASSOCIATED_EVENTS_DURING_G2_M_TRANSITION | 7 | 0 | 0.048679654 | 0.215 |

TABLE 8
Pathways significantly (FDR 5%) associated with increase in protein abundance
levels upon Compound A or lenalidomide exposure in Case B3

| CONDITION | NAME | SIZE | NOM.p.val | FDR.q.val | FWER.p.val |
|---|---|---|---|---|---|
| len_10uM_24h | REACTOME_INTERFERON_GAMMA_SIGNALING | 37 | 0 | 0 | 0 |
| len_10uM_24h | REACTOME_CYTOKINE_SIGNALING_IN_IMMUNE_SYSTEM | 158 | 0 | 0 | 0 |
| len_10uM_24h | REACTOME_INTERFERON_SIGNALING | 100 | 0 | 0 | 0 |
| len_10uM_24h | REACTOME_INTERFERON_ALPHA_BETA_SIGNALING | 27 | 0 | 0 | 0 |
| len_10uM_24h | KEGG_ALLOGRAFT_REJECTION | 19 | 0 | 0.00046156909 | 0.002 |
| len_10uM_24h | REACTOME_NUCLEOTIDE_BINDING_DOMAIN_LEUCINE_RICH_REPEAT_{—} | 26 | 0 | 0.0005538829 | 0.002 |
| | CONTAINING_RECEPTOR_NLR_SIGNALING_PATHWAYS | | | | |
| len_10uM_24h | REACTOME_IMMUNE_SYSTEM | 487 | 0 | 0.0005933928 | 0.003 |
| len_10uM_24h | KEGG_GRAFT_VERSUS_HOST_DISEASE | 17 | 0 | 0.0008688712 | 0.005 |
| len_10uM_24h | REACTOME_NOD1_2_SIGNALING_PATHWAY | 17 | 0 | 0.0009301702 | 0.006 |
| len_10uM_24h | KEGG_FC_EPSILON_RI_SIGNALING_PATHWAY | 44 | 0 | 0.0009786802 | 0.007 |
| len_10uM_24h | REACTOME_SIGNALING_BY_RHO_GTPASES | 59 | 0 | 0.0010901617 | 0.011 |
| len_10uM_24h | KEGG_VIRAL_MYOCARDITIS | 39 | 0 | 0.0011081224 | 0.012 |
| len_10uM_24h | KEGG_NATURAL_KILLER_CELL_MEDIATED_CYTOTOXICITY | 54 | 0 | 0.0011595621 | 0.01 |
| len_10uM_24h | PID_IL12_2PATHWAY | 26 | 0 | 0.0011740203 | 0.011 |
| len_10uM_24h | KEGG_AUTOIMMUNE_THYROID_DISEASE | 19 | 0 | 0.0012649769 | 0.01 |
| len_10uM_24h | PID_FCER1PATHWAY | 44 | 0 | 0.0018317524 | 0.021 |
| len_10uM_24h | REACTOME_SIGNALING_BY_ILS | 63 | 0 | 0.0032483914 | 0.041 |
| len_10uM_24h | REACTOME_INNATE_IMMUNE_SYSTEM | 110 | 0 | 0.0033578933 | 0.04 |
| len_10uM_24h | KEGG_FC_GAMMA_R_MEDIATED_PHAGOCYTOSIS | 60 | 0 | 0.003524156 | 0.047 |
| len_10uM_24h | PID_KITPATHWAY | 34 | 0 | 0.003769228 | 0.053 |
| len_10uM_24h | PID_TCR_PATHWAY | 43 | 0 | 0.0040012607 | 0.062 |
| len_10uM_24h | PID_RAC1_PATHWAY | 41 | 0 | 0.004057631 | 0.06 |
| len_10uM_24h | KEGG_TYPE_I_DIABETES_MELLITUS | 19 | 0 | 0.0041892016 | 0.071 |
| len_10uM_24h | REACTOME_IMMUNOREGULATORY_INTERACTIONS_BETWEEN_A_LYMPHOID_AND_A_NON_LYMPHOID_CELL | 18 | 0 | 0.0042536953 | 0.069 |
| len_10uM_24h | PID_EPOPATHWAY | 27 | 0 | 0.0063125407 | 0.101 |
| len_10uM_24h | PID_HIVNEFPATHWAY | 24 | 0.0022371365 | 0.007952232 | 0.131 |
| len_10uM_24h | PID_TNFPATHWAY | 35 | 0 | 0.007986917 | 0.142 |
| len_10uM_24h | REACTOME_ENDOSOMAL_VACUOLAR_PATHWAY | 7 | 0 | 0.0082827285 | 0.142 |
| len_10uM_24h | KEGG_GLUTATHIONE_METABOLISM | 31 | 0.0023310024 | 0.00857727 | 0.158 |
| len_10uM_24h | PID_CDC42_PATHWAY | 53 | 0 | 0.008897451 | 0.17 |
| len_10uM_24h | SA_CASPASE_CASCADE | 10 | 0 | 0.0091802515 | 0.187 |
| len_10uM_24h | PID_FASPATHWAY | 30 | 0 | 0.009384733 | 0.185 |
| len_10uM_24h | PID_GMCSF_PATHWAY | 27 | 0 | 0.010021957 | 0.237 |
| len_10uM_24h | KEGG_TOLL_LIKE_RECEPTOR_SIGNALING_PATHWAY | 43 | 0 | 0.010079143 | 0.22 |
| len_10uM_24h | KEGG_CELL_ADHESION_MOLECULES_CAMS | 36 | 0 | 0.010149377 | 0.228 |
| len_10uM_24h | REACTOME_METABOLISM_OF_NUCLEOTIDES | 48 | 0 | 0.010179714 | 0.211 |
| len_10uM_24h | KEGG_LEUKOCYTE_TRANSENDOTHELIAL_MIGRATION | 59 | 0 | 0.01025506 | 0.236 |
| len_10uM_24h | REACTOME_SIGNALING_BY_SCF_KIT | 47 | 0 | 0.010291103 | 0.219 |
| len_10uM_24h | REACTOME_DOUBLE_STRAND_BREAK_REPAIR | 17 | 0.0023696683 | 0.012949456 | 0.303 |
| len_10uM_24h | KEGG_B_CELL_RECEPTOR_SIGNALING_PATHWAY | 54 | 0 | 0.01300675 | 0.339 |
| len_10uM_24h | PID_CD8TCRPATHWAY | 31 | 0.0022883294 | 0.0131391315 | 0.32 |
| len_10uM_24h | REACTOME_GENERATION_OF_SECOND_MESSENGER_MOLECULES | 14 | 0.0045454544 | 0.013225859 | 0.329 |
| len_10uM_24h | BIOCARTA_MAPK_PATHWAY | 58 | 0 | 0.013275946 | 0.338 |
| len_10uM_24h | REACTOME_IL_2_SIGNALING | 30 | 0 | 0.013294971 | 0.317 |
| len_10uM_24h | PID_BCR_5PATHWAY | 53 | 0 | 0.017718375 | 0.433 |
| len_10uM_24h | PID_PI3KCIPATHWAY | 34 | 0.002386635 | 0.018068314 | 0.453 |
| len_10uM_24h | BIOCARTA_HIVNEF_PATHWAY | 42 | 0 | 0.018089063 | 0.445 |
| len_10uM_24h | REACTOME_ADAPTIVE_IMMUNE_SYSTEM | 310 | 0 | 0.018216273 | 0.462 |
| len_10uM_24h | BIOCARTA_CASPASE_PATHWAY | 17 | 0.002283105 | 0.018868817 | 0.481 |
| len_10uM_24h | REACTOME_PURINE_SALVAGE | 10 | 0 | 0.019662427 | 0.501 |
| len_10uM_24h | REACTOME_COSTIMULATION_BY_THE_CD28_FAMILY | 37 | 0.004672897 | 0.021746496 | 0.547 |
| len_10uM_24h | PID_CASPASE_PATHWAY | 36 | 0 | 0.021748602 | 0.564 |
| len_10uM_24h | PID_P38_MKK3_6PATHWAY | 16 | 0.0023094688 | 0.022107158 | 0.564 |
| len_10uM_24h | ST_TUMOR_NECROSIS_FACTOR_PATHWAY | 19 | 0 | 0.02221318 | 0.559 |
| len_10uM_24h | SIG_BCR_SIGNALING_PATHWAY | 39 | 0 | 0.023782676 | 0.603 |
| len_10uM_24h | REACTOME_G2_M_CHECKPOINTS | 26 | 0.0047281324 | 0.027753348 | 0.67 |
| len_10uM_24h | REACTOME_KINESINS | 18 | 0 | 0.029282661 | 0.693 |
| len_10uM_24h | BIOCARTA_NKCELLS_PATHWAY | 13 | 0 | 0.03151 | 0.732 |
| len_10uM_24h | PID_MAPKTRKPATHWAY | 21 | 0 | 0.031951945 | 0.748 |
| len_10uM_24h | REACTOME_PHASE_II_CONJUGATION | 24 | 0.0043668123 | 0.03198237 | 0.756 |
| len_10uM_24h | KEGG_CHEMOKINE_SIGNALING_PATHWAY | 84 | 0 | 0.032995272 | 0.783 |
| len_10uM_24h | BIOCARTA_GLEEVEC_PATHWAY | 18 | 0.006960557 | 0.03313099 | 0.774 |
| len_10uM_24h | PID_CD40_PATHWAY | 23 | 0 | 0.033162586 | 0.78 |
| len_10uM_24h | KEGG_RIG_I_LIKE_RECEPTOR_SIGNALING_PATHWAY | 33 | 0.004854369 | 0.033468146 | 0.795 |
| len_10uM_24h | REACTOME_EXTRINSIC_PATHWAY_FOR_APOPTOSIS | 5 | 0 | 0.03570184 | 0.826 |
| len_10uM_24h | BIOCARTA_HCMV_PATHWAY | 15 | 0 | 0.038035948 | 0.851 |
| len_10uM_24h | BIOCARTA_IL3_PATHWAY | 11 | 0.004784689 | 0.03914031 | 0.868 |
| len_10uM_24h | REACTOME_NFKB_ACTIVATION_THROUGH_FADD_RIP1_PATHWAY_MEDIATED_BY_CASPASE_8_AND10 | 9 | 0.006355932 | 0.03930227 | 0.869 |
| len_10uM_24h | BIOCARTA_SODD_PATHWAY | 6 | 0.006651885 | 0.040025186 | 0.875 |
| len_10uM_24h | BIOCARTA_BLYMPHOCYTE_PATHWAY | 8 | 0.0065075923 | 0.04045706 | 0.887 |
| len_10uM_24h | PID_IFNGPATHWAY | 30 | 0.0022522523 | 0.040952433 | 0.887 |
| len_10uM_24h | PID_RHOA_REG_PATHWAY | 25 | 0.004474273 | 0.042852033 | 0.901 |
| len_10uM_24h | KEGG_CYSTEINE_AND_METHIONINE_METABOLISM | 20 | 0.004842615 | 0.04732237 | 0.925 |
| len_10uM_24h | PID_BARD1PATHWAY | 15 | 0.0043290043 | 0.048030593 | 0.937 |
| len_10uM_24h | REACTOME_PURINE_METABOLISM | 27 | 0.0022371365 | 0.04825746 | 0.931 |
| len_10uM_24h | REACTOME_HOMOLOGOUS_RECOMBINATION_REPAIR_OF_REPLICATION_INDEPENDENT_{—} | 11 | 0.015555556 | 0.048341785 | 0.932 |
| | DOUBLE_STRAND_BREAKS | | | | |
| len_10uM_24h | PID_RAC1_REG_PATHWAY | 25 | 0.004672897 | 0.048460755 | 0.937 |
| len_10uM_24h | SIG_INSULIN_RECEPTOR_PATHWAY_IN_CARDIAC_MYOCYTES | 39 | 0.0072289156 | 0.048582643 | 0.941 |
| len_10uM_24h | REACTOME_TCR_SIGNALING | 31 | 0.0073349634 | 0.049583502 | 0.947 |
| CmpA_10uM_24h | REACTOME_INTERFERON_SIGNALING | 100 | 0 | 0 | 0 |
| CmpA_10uM_24h | REACTOME_INTERFERON_GAMMA_SIGNALING | 37 | 0 | 0 | 0 |
| CmpA_10uM_24h | REACTOME_CYTOKINE_SIGNALING_IN_IMMUNE_SYSTEM | 158 | 0 | 0 | 0 |
| CmpA_10uM_24h | REACTOME_INTERFERON_ALPHA_BETA_SIGNALING | 27 | 0 | 0 | 0 |
| CmpA_10uM_24h | KEGG_TYPE_I_DIABETES_MELLITUS | 19 | 0 | 9.49E+02 | 0.001 |
| CmpA_10uM_24h | REACTOME_IMMUNE_SYSTEM | 487 | 0 | 0.00010357675 | 0.001 |
| CmpA_10uM_24h | KEGG_CELL_ADHESION_MOLECULES_CAMS | 36 | 0 | 0.000113934424 | 0.001 |
| CmpA_10uM_24h | KEGG_ANTIGEN_PROCESSING_AND_PRESENTATION | 43 | 0 | 0.0001265938 | 0.001 |
| CmpA_10uM_24h | KEGG_GRAFT_VERSUS_HOST_DISEASE | 17 | 0 | 0.00014241802 | 0.001 |
| CmpA_10uM_24h | KEGG_AUTOIMMUNE_THYROID_DISEASE | 19 | 0 | 0.00016276345 | 0.001 |
| CmpA_10uM_24h | KEGG_ALLOGRAFT_REJECTION | 19 | 0 | 0.00018989069 | 0.001 |
| CmpA_10uM_24h | KEGG_VIRAL_MYOCARDITIS | 39 | 0 | 0.00022786885 | 0.001 |
| CmpA_10uM_24h | KEGG_SYSTEMIC_LUPUS_ERYTHEMATOSUS | 39 | 0 | 0.00026754476 | 0.003 |
| CmpA_10uM_24h | KEGG_NATURAL_KILLER_CELL_MEDIATED_CYTOTOXICITY | 54 | 0 | 0.0004954588 | 0.006 |
| CmpA_10uM_24h | PID_CD8TCRPATHWAY | 31 | 0 | 0.00053986546 | 0.007 |
| CmpA_10uM_24h | REACTOME_ANTIGEN_PROCESSING_CROSS_PRESENTATION | 62 | 0 | 0.0008755847 | 0.012 |
| CmpA_10uM_24h | REACTOME_DOUBLE_STRAND_BREAK_REPAIR | 17 | 0 | 0.0011689598 | 0.017 |
| CmpA_10uM_24h | KEGG_FC_GAMMA_R_MEDIATED_PHAGOCYTOSIS | 60 | 0 | 0.0013020117 | 0.02 |
| CmpA_10uM_24h | REACTOME_NUCLEOTIDE_BINDING_DOMAIN_LEUCINE_RICH_{—} | 26 | 0 | 0.00252846 | 0.039 |
| | REPEAT_CONTAINING_RECEPTOR_NLR_SIGNALING_PATHWAYS | | | | |
| CmpA_10uM_24h | KEGG_GLUTATHIONE_METABOLISM | 31 | 0 | 0.0026202581 | 0.045 |
| CmpA_10uM_24h | KEGG_INTESTINAL_IMMUNE_NETWORK_FOR_IGA_PRODUCTION | 15 | 0 | 0.0026586454 | 0.047 |
| CmpA_10uM_24h | PID_TCR_PATHWAY | 43 | 0 | 0.0026925385 | 0.044 |
| CmpA_10uM_24h | PID_IL12_2PATHWAY | 26 | 0 | 0.0038203301 | 0.069 |
| CmpA_10uM_24h | KEGG_LEUKOCYTE_TRANSENDOTHELIAL_MIGRATION | 59 | 0 | 0.0042452407 | 0.08 |
| CmpA_10uM_24h | KEGG_FC_EPSILON_RI_SIGNALING_PATHWAY | 44 | 0.0015923567 | 0.0075306506 | 0.147 |
| CmpA_10uM_24h | PID_TNFPATHWAY | 35 | 0.0016129032 | 0.007832585 | 0.157 |
| CmpA_10uM_24h | REACTOME_ANTIGEN_PRESENTATION_FOLDING_ASSEMBLY_AND_PEPTIDE_LOADING_OF_CLASS_I_MHC | 19 | 0 | 0.008576823 | 0.178 |
| CmpA_10uM_24h | REACTOME_SIGNALING_BY_RHO_GTPASES | 59 | 0 | 0.009360026 | 0.197 |
| CmpA_10uM_24h | KEGG_ASTHMA | 12 | 0 | 0.011703992 | 0.248 |
| CmpA_10uM_24h | REACTOME_ER_PHAGOSOME_PATHWAY | 54 | 0 | 0.013929606 | 0.303 |
| CmpA_10uM_24h | SA_CASPASE_CASCADE | 10 | 0 | 0.0140437065 | 0.298 |
| CmpA_10uM_24h | REACTOME_INNATE_IMMUNE_SYSTEM | 110 | 0 | 0.014698049 | 0.322 |
| CmpA_10uM_24h | REACTOME_ENDOSOMAL_VACUOLAR_PATHWAY | 7 | 0 | 0.015277403 | 0.343 |
| CmpA_10uM_24h | KEGG_TOLL_LIKE_RECEPTOR_SIGNALING_PATHWAY | 43 | 0 | 0.016813265 | 0.378 |
| CmpA_10uM_24h | BIOCARTA_BLYMPHOCYTE_PATHWAY | 8 | 0.001953125 | 0.017601717 | 0.406 |
| CmpA_10uM_24h | REACTOME_NOD1_2_SIGNALING_PATHWAY | 17 | 0 | 0.0205842 | 0.464 |
| CmpA_10uM_24h | REACTOME_NFKB_ACTIVATION_THROUGH_FADD_RIP1_PATHWAY_MEDIATED_BY_CASPASE_8_AND10 | 9 | 0.0019493178 | 0.020759648 | 0.473 |
| CmpA_10uM_24h | PID_IL12_STAT4PATHWAY | 14 | 0.0018416207 | 0.021136621 | 0.487 |
| CmpA_10uM_24h | KEGG_LEISHMANIA_INFECTION | 36 | 0 | 0.021758901 | 0.505 |
| CmpA_10uM_24h | PID_ERBB1_DOWNSTREAM_PATHWAY | 77 | 0 | 0.022006301 | 0.519 |
| CmpA_10uM_24h | REACTOME_COSTIMULATION_BY_THE_CD28_FAMILY | 37 | 0 | 0.023410125 | 0.557 |
| CmpA_10uM_24h | REACTOME_PHOSPHORYLATION_OF_CD3_AND_TCR_ZETA_CHAINS | 8 | 0.001953125 | 0.025384363 | 0.598 |
| CmpA_10uM_24h | REACTOME_GENERATION_OF_SECOND_MESSENGER_MOLECULES | 14 | 0.0056603774 | 0.030597936 | 0.673 |
| CmpA_10uM_24h | KEGG_COMPLEMENT_AND_COAGULATION_CASCADES | 18 | 0.0017513135 | 0.031897366 | 0.699 |
| CmpA_10uM_24h | BIOCARTA_CTLA4_PATHWAY | 8 | 0.0018552876 | 0.035306435 | 0.736 |
| CmpA_10uM_24h | PID_CASPASE_PATHWAY | 36 | 0.0032573289 | 0.042535875 | 0.793 |
| CmpA_10uM_24h | REACTOME_ADAPTIVE_IMMUNE_SYSTEM | 310 | 0 | 0.043032773 | 0.803 |
| CmpA_10uM_24h | PID_RHOA_REG_PATHWAY | 25 | 0.0102739725 | 0.04315912 | 0.792 |
| CmpA_10uM_24h | PID_FASPATHWAY | 30 | 0.004761905 | 0.044866573 | 0.847 |
| CmpA_10uM_24h | BIOCARTA_MITOCHONDRIA_PATHWAY | 17 | 0.007259528 | 0.045482043 | 0.843 |
| CmpA_10uM_24h | PID_RAC1_REG_PATHWAY | 25 | 0.0034423408 | 0.04550309 | 0.837 |
| CmpA_10uM_24h | SIG_BCR_SIGNALING_PATHWAY | 39 | 0.0015197569 | 0.045662835 | 0.833 |
| CmpA_10uM_24h | REACTOME_PD1_SIGNALING | 8 | 0.0018691589 | 0.04640493 | 0.833 |
| CmpA_10uM_24h | KEGG_REGULATION_OF_ACTIN_CYTOSKELETON | 104 | 0 | 0.046439912 | 0.864 |

TABLE 9
Pathways significantly (FDR 5%) associated with increase in protein abundance
levels upon Compound A or lenalidomide exposure in Case B4

| Condition | Name | Size | NOM.p.val | FDR.q.val | FWER.p.val |
|---|---|---|---|---|---|
| CmpA_10uM_24h | KEGG_CELL_ADHESION_MOLECULES_CAMS | 31 | 0 | 0 | 0 |
| CmpA_10uM_24h | PID_LYSOPHOSPHOLIPID_PATHWAY | 38 | 0 | 0.009543243 | 0.018 |
| CmpA_10uM_24h | PID_ENDOTHELINPATHWAY | 31 | 0.0018975332 | 0.048976257 | 0.401 |

Tables 10-13 show pathways significantly associated with decreased protein abundance upon compound exposure.

TABLE 10
Pathways significantly (FDR 5%) associated with decrease in protein abundance
levels upon Compound A or lenalidomide exposure in Case B1

| Condition | Name | Size | NOM.p.val | FDR.q.val | FWER.p.val |
|---|---|---|---|---|---|
| len_10uM_24h | KEGG_LYSOSOME | 70 | 0 | 0.0027681112 | 0.003 |
| len_10uM_24h | REACTOME_TRANSMEMBRANE_TRANSPORT_OF_SMALL_{—} | 121 | 0 | 0.033620406 | 0.069 |
| | MOLECULES | | | | |
| len_10uM_24h | REACTOME_PLATELET_HOMEOSTASIS | 31 | 0 | 0.034212988 | 0.165 |
| len_10uM_24h | REACTOME_CTNNB1_PHOSPHORYLATION_CASCADE | 10 | 0 | 0.035863783 | 0.107 |
| len_10uM_24h | REACTOME_PLATELET_SENSITIZATION_BY_LDL | 13 | 0 | 0.04108309 | 0.159 |
| len_10uM_24h | REACTOME_ABC_FAMILY_PROTEINS_MEDIATED_TRANSPORT | 11 | 0 | 0.049495384 | 0.26 |

TABLE 11
Pathways significantly (FDR 5%) associated with decrease in protein
abundance levels upon Compound A or lenalidomide exposure in Case B3

| Condition | Name | Size | NOM.p.val | FDR.q.val | FWER.p.val |
|---|---|---|---|---|---|
| len_10uM_24h | REACTOME_TRANSMEMBRANE_TRANSPORT_OF_SMALL_MOLECULES | 112 | 0 | 0 | 0 |
| len_10uM_24h | REACTOME_MITOCHONDRIAL_PROTEIN_IMPORT | 38 | 0 | 0 | 0 |
| len_10uM_24h | KEGG_LYSOSOME | 71 | 0 | 0.00016678323 | 0.001 |
| len_10uM_24h | REACTOME_TCA_CYCLE_AND_RESPIRATORY_ELECTRON_TRANSPORT | 100 | 0 | 0.00019168781 | 0.002 |
| len_10uM_24h | REACTOME_PHASE1_FUNCTIONALIZATION_OF_COMPOUNDS | 12 | 0 | 0.00022237762 | 0.001 |
| len_10uM_24h | REACTOME_PYRUVATE_METABOLISM_AND_CITRIC_ACID_TCA_CYCLE | 37 | 0 | 0.00022363578 | 0.002 |
| len_10uM_24h | REACTOME_SLC_MEDIATED_TRANSMEMBRANE_TRANSPORT | 57 | 0 | 0.00026836296 | 0.002 |
| len_10uM_24h | REACTOME_SPHINGOLIPID_METABOLISM | 27 | 0 | 0.00030020459 | 0.004 |
| len_10uM_24h | REACTOME_GLYCOSAMINOGLYCAN_METABOLISM | 21 | 0 | 0.00033773016 | 0.004 |
| len_10uM_24h | KEGG_OTHER_GLYCAN_DEGRADATION | 13 | 0 | 0.0003384245 | 0.005 |
| len_10uM_24h | KEGG_N_GLYCAN_BIOSYNTHESIS | 30 | 0 | 0.00086016697 | 0.015 |
| len_10uM_24h | REACTOME_EXTRACELLULAR_MATRIX_ORGANIZATION | 20 | 0 | 0.0008757902 | 0.014 |
| len_10uM_24h | REACTOME_METABOLISM_OF_LIPIDS_AND_LIPOPROTEINS | 216 | 0 | 0.0008998296 | 0.017 |
| len_10uM_24h | REACTOME_POST_TRANSLATIONAL_PROTEIN_MODIFICATION | 75 | 0 | 0.0012891265 | 0.026 |
| len_10uM_24h | REACTOME_GLYCOSPHINGOLIPID_METABOLISM | 16 | 0 | 0.0021206257 | 0.046 |
| len_10uM_24h | REACTOME_TRANSFERRIN_ENDOCYTOSIS_AND_RECYCLING | 15 | 0 | 0.002232281 | 0.055 |
| len_10uM_24h | REACTOME_COLLAGEN_FORMATION | 18 | 0 | 0.0023298683 | 0.054 |
| len_10uM_24h | REACTOME_IRON_UPTAKE_AND_TRANSPORT | 19 | 0.0018281536 | 0.0027933633 | 0.072 |
| len_10uM_24h | REACTOME_UNFOLDED_PROTEIN_RESPONSE | 54 | 0 | 0.005160602 | 0.14 |
| len_10uM_24h | REACTOME_TRANSPORT_OF_INORGANIC_CATIONS_ANIONS_AND_AMINO_ACIDS_OLIGOPEPTIDES | 16 | 0 | 0.005396703 | 0.14 |
| len_10uM_24h | KEGG_OXIDATIVE_PHOSPHORYLATION | 84 | 0 | 0.006099407 | 0.177 |
| len_10uM_24h | REACTOME_ACTIVATION_OF_CHAPERONE_GENES_BY_XBP1S | 34 | 0 | 0.006124594 | 0.17 |
| len_10uM_24h | KEGG_ECM_RECEPTOR_INTERACTION | 24 | 0.0019047619 | 0.0061616744 | 0.195 |
| len_10uM_24h | REACTOME_LIPID_DIGESTION_MOBILEATION_AND_TRANSPORT | 16 | 0.0017730496 | 0.0062507736 | 0.189 |
| len_10uM_24h | REACTOME_CYTOCHROME_P450_ARRANGED_BY_SUBSTRATE_TYPE | 6 | 0 | 0.006271437 | 0.208 |
| len_10uM_24h | REACTOME_PYRUVATE_METABOLISM | 16 | 0.0035971224 | 0.0068569663 | 0.232 |
| len_10uM_24h | KEGG_SPHINGOLIPID_METABOLISM | 13 | 0 | 0.00852841 | 0.289 |
| len_10uM_24h | KEGG_CITRATE_CYCLE_TCA_CYCLE | 27 | 0 | 0.00871896 | 0.3 |
| len_10uM_24h | REACTOME_ASPARAGINE_N_LINKED_GLYCOSYLATION | 55 | 0 | 0.012145675 | 0.397 |
| len_10uM_24h | REACTOME_FATTY_ACID_TRIACYLGLYCEROL_AND_KETONE_BODY_METABOLISM | 96 | 0 | 0.013621413 | 0.444 |
| len_10uM_24h | REACTOME_CITRIC_ACID_CYCLE_TCA_CYCLE | 19 | 0.0035149385 | 0.014275145 | 0.471 |
| len_10uM_24h | REACTOME_CHONDROITIN_SULFATE_DERMATAN_SULFATE_METABOLISM | 7 | 0 | 0.0160844 | 0.525 |
| len_10uM_24h | REACTOME_INSULIN_RECEPTOR_RECYCLING | 13 | 0.003552398 | 0.016285133 | 0.538 |
| len_10uM_24h | REACTOME_REGULATION_OF_PYRUVATE_DEHYDROGENASE_PDH_COMPLEX | 11 | 0.0035778175 | 0.017164052 | 0.566 |
| len_10uM_24h | KEGG_VIBRIO_CHOLERAE_INFECTION | 31 | 0.0017730496 | 0.021985121 | 0.69 |
| len_10uM_24h | REACTOME_PEROXISOMAL_LIPID_METABOLISM | 15 | 0 | 0.02198896 | 0.707 |
| len_10uM_24h | REACTOME_RESPIRATORY_ELECTRON_TRANSPORT | 56 | 0 | 0.022087615 | 0.661 |
| len_10uM_24h | REACTOME_ABC_FAMILY_PROTEINS_MEDIATED_TRANSPORT | 11 | 0 | 0.022302283 | 0.685 |
| len_10uM_24h | REACTOME_LIPOPROTEIN_METABOLISM | 11 | 0.0017667845 | 0.022535129 | 0.706 |
| len_10uM_24h | REACTOME_RESPIRATORY_ELECTRON_TRANSPORT_ATP_SYNTHESIS_BY_CHEMIOSMOTIC_COUPLING_AND_{—} | 67 | 0 | 0.022633191 | 0.68 |
| | HEAT_PRODUCTION_BY_UNCOUPLING_PROTEINS_{—} | | | | |
| len_10uM_24h | REACTOME_INTERACTIONS_OF_VPR_WITH_HOST_CELLULAR_PROTEINS | 32 | 0.0038022813 | 0.0235497 | 0.745 |
| len_10uM_24h | REACTOME_BIOLOGICAL_OXIDATIONS | 36 | 0.0017123288 | 0.02654474 | 0.793 |
| len_10uM_24h | KEGG_PARKINSONS_DISEASE | 81 | 0.0017482517 | 0.02866812 | 0.829 |
| len_10uM_24h | REACTOME_RNA_POL_III_TRANSCRIPTION_INITIATION_FROM_TYPE_2_PROMOTER | 17 | 0.0036231885 | 0.02880658 | 0.824 |
| len_10uM_24h | REACTOME_TRANSPORT_OF_RIBONUCLEOPROTEINS_INTO_THE_HOST_NUCLEUS | 27 | 0.00877193 | 0.031250864 | 0.865 |
| len_10uM_24h | REACTOME_PPARA_ACTIVATES_GENE_EXPRESSION | 49 | 0 | 0.03161707 | 0.864 |
| len_10uM_24h | REACTOME_DIABETES_PATHWAYS | 78 | 0.0017421603 | 0.031789344 | 0.89 |
| len_10uM_24h | REACTOME_AMINO_ACID_AND_OLIGOPEPTIDE_SLC_TRANSPORTERS | 8 | 0.001776199 | 0.03193902 | 0.875 |
| len_10uM_24h | REACTOME_HEPARAN_SULFATE_HEPARIN_HS_GAG_METABOLISM | 11 | 0.007707129 | 0.032241717 | 0.888 |
| len_10uM_24h | REACTOME_TRANSPORT_OF_MATURE_MRNA_DERIVED_FROM_AN_INTRONLESS_TRANSCRIPT | 32 | 0.0055555557 | 0.03228689 | 0.897 |
| len_10uM_24h | REACTOME_ION_CHANNEL_TRANSPORT | 11 | 0.006932409 | 0.032319672 | 0.864 |
| len_10uM_24h | KEGG_HYPERTROPHIC_CARDIOMYOPATHY_HCM | 23 | 0.005291005 | 0.0329675 | 0.901 |
| len_10uM_24h | REACTOME_TRANSPORT_OF_GLUCOSE_AND_OTHER_SUGARS_BILE_SALTS_AND_ORGANIC_ACIDS_METAL_{—} | 11 | 0.005504587 | 0.033189867 | 0.906 |
| | IONS_AND_AMINE_COMPOUNDS | | | | |
| len_10uM_24h | REACTOME_GLUCOSE_TRANSPORT | 28 | 0.0034364262 | 0.033783562 | 0.918 |
| len_10uM_24h | ST_WNT_BETA_CATENIN_PATHWAY | 13 | 0.011090573 | 0.038872246 | 0.957 |
| len_10uM_24h | REACTOME_ION_TRANSPORT_BY_P_TYPE_ATPASES | 11 | 0.00754717 | 0.03903826 | 0.961 |
| len_10uM_24h | KEGG_DILATED_CARDIOMYOPATHY | 21 | 0.0053859963 | 0.039395284 | 0.964 |
| len_10uM_24h | REACTOME_POST_TRANSLATIONAL_MODIFICATION_SYNTHESIS_OF_GPI_ANCHORED_PROTEINS | 8 | 0 | 0.03943385 | 0.957 |
| len_10uM_24h | KEGG_ALZHEIMERS_DISEASE | 103 | 0 | 0.039677624 | 0.954 |
| len_10uM_24h | REACTOME_REGULATION_OF_GLUCOKINASE_BY_GLUCOKINASE_REGULATORY_PROTEIN | 25 | 0.0053097345 | 0.039904807 | 0.955 |
| len_10uM_24h | REACTOME_MRNA_SPLICING_MINOR_PATHWAY | 39 | 0.009090909 | 0.046694405 | 0.983 |
| len_10uM_24h | KEGG_GLYCOSYLPHOSPHATIDYLINOSITOL_GPI_ANCHOR_BIOSYNTHESIS | 6 | 0 | 0.0471724 | 0.983 |
| len_10uM_24h | KEGG_ABC_TRANSPORTERS | 15 | 0.0102739725 | 0.04736988 | 0.98 |
| len_10uM_24h | REACTOME_PHOSPHOLIPID_METABOLISM | 90 | 0 | 0.04747753 | 0.982 |
| len_10uM_24h | PID_SYNDECAN_1_PATHWAY | 16 | 0.007490637 | 0.04961539 | 0.988 |
| len_10uM_24h | REACTOME_AMINO_ACID_TRANSPORT_ACROSS_THE_PLASMA_MEMBRANE | 7 | 0.009259259 | 0.04997752 | 0.988 |

TABLE 12
Pathways significantly (FDR 5%) associated with decrease in protein
abundance levels upon Compound A or lenalidomide exposure in Case B4

| CONDITION | NAME | SIZE | NOM.p.val | FDR.q.val | FWER.p.val |
|---|---|---|---|---|---|
| len_10uM_24h | KEGG_PENTOSE_PHOSPHATE_PATHWAY | 19 | 0 | 0.014535695 | 0.014 |
| len_10uM_24h | REACTOME_GLYCOLYSIS | 21 | 0 | 0.01667701 | 0.076 |
| len_10uM_24h | KEGG_COMPLEMENT_AND_COAGULATION_CASCADES | 17 | 0 | 0.018007392 | 0.066 |
| len_10uM_24h | REACTOME_PURINE_METABOLISM | 26 | 0 | 0.02332448 | 0.064 |
| len_10uM_24h | REACTOME_METABOLISM_OF_CARBOHYDRATES | 102 | 0 | 0.024844436 | 0.15 |
| len_10uM_24h | KEGG_FRUCTOSE_AND_MANNOSE_METABOLISM | 25 | 0 | 0.026381705 | 0.137 |
| len_10uM_24h | REACTOME_METABOLISM_OF_NUCLEOTIDES | 49 | 0 | 0.031843547 | 0.059 |
| len_10uM_24h | KEGG_STARCH_AND_SUCROSE_METABOLISM | 17 | 0 | 0.03490622 | 0.231 |
| len_10uM_24h | REACTOME_GLUCOSE_METABOLISM | 44 | 0 | 0.04062623 | 0.292 |
| CmpA_10uM_24h | REACTOME_GLYCOLYSIS | 21 | 0 | 0.0014749501 | 0.008 |
| CmpA_10uM_24h | REACTOME_REGULATION_OF_ORNITHINE_DECARBOXYLASE_ODC | 42 | 0 | 0.0017060097 | 0.011 |
| CmpA_10uM_24h | REACTOME_ACTIVATION_OF_THE_MRNA_UPON_BINDING_OF_THE_CAP_BINDING_COMPLEX_AND_{—} | 53 | 0 | 0.0018436876 | 0.008 |
| | EIFS_AND_SUBSEQUENT_BINDING_TO_43S | | | | |
| CmpA_10uM_24h | REACTOME_METABOLISM_OF_NUCLEOTIDES | 49 | 0 | 0.0024582502 | 0.008 |
| CmpA_10uM_24h | REACTOME_FORMATION_OF_THE_TERNARY_COMPLEX_AND_SUBSEQUENTLY_THE_43S_COMPLEX | 46 | 0 | 0.0027701426 | 0.006 |
| CmpA_10uM_24h | KEGG_PURINE_METABOLISM | 72 | 0 | 0.0034616417 | 0.026 |
| CmpA_10uM_24h | KEGG_FRUCTOSE_AND_MANNOSE_METABOLISM | 25 | 0 | 0.0037304503 | 0.004 |
| CmpA_10uM_24h | REACTOME_APC_C_CDH1_MEDIATED_DEGRADATION_OF_CDC20_AND_OTHER_APC_C_CDH1_TARGETED_{—} | 53 | 0 | 0.0037335916 | 0.036 |
| | PROTEINS_IN_LATE_MITOSIS_EARLY_G1 | | | | |
| CmpA_10uM_24h | KEGG_PROTEASOME | 39 | 0.0020920502 | 0.0038449848 | 0.033 |
| CmpA_10uM_24h | REACTOME_ANTIGEN_PROCESSING_UBIQUITINATION_PROTEASOME_DEGRADATION | 119 | 0 | 0.004170974 | 0.044 |
| CmpA_10uM_24h | REACTOME_REGULATION_OF_APOPTOSIS | 47 | 0 | 0.006089002 | 0.088 |
| CmpA_10uM_24h | REACTOME_CDK_MEDIATED_PHOSPHORYLATION_AND_REMOVAL_OF_CDC6 | 43 | 0 | 0.006168002 | 0.076 |
| CmpA_10uM_24h | REACTOME_DESTABILIZATION_OF_MRNA_BY_AUF1_HNRNP_D0 | 47 | 0 | 0.00629705 | 0.098 |
| CmpA_10uM_24h | REACTOME_AUTODEGRADATION_OF_CDH1_BY_CDH1_APC_C | 50 | 0 | 0.0063076103 | 0.072 |
| CmpA_10uM_24h | REACTOME_CROSS_PRESENTATION_OF_SOLUBLE_EXOGENOUS_ANTIGENS_ENDOSOMES | 42 | 0 | 0.00631505 | 0.104 |
| CmpA_10uM_24h | REACTOME_METABOLISM_OF_CARBOHYDRATES | 102 | 0 | 0.0064845774 | 0.087 |
| CmpA_10uM_24h | REACTOME_APC_C_CDC20_MEDIATED_DEGRADATION_OF_MITOTIC_PROTEINS | 54 | 0 | 0.007525836 | 0.128 |
| CmpA_10uM_24h | REACTOME_P53_INDEPENDENT_G1_S_DNA_DAMAGE_CHECKPOINT | 43 | 0 | 0.009707467 | 0.193 |
| CmpA_10uM_24h | REACTOME_AUTODEGRADATION_OF_THE_E3_UBIQUITIN_LIGASE_COP1 | 44 | 0 | 0.009952889 | 0.178 |
| CmpA_10uM_24h | REACTOME_CYTOSOLIC_TRNA_AMINOACYLATION | 24 | 0 | 0.010218386 | 0.193 |
| CmpA_10uM_24h | BIOCARTA_PROTEASOME_PATHWAY | 28 | 0 | 0.011101849 | 0.229 |
| CmpA_10uM_24h | REACTOME_SCF_BETA_TRCP_MEDIATED_DEGRADATION_OF_EMI1 | 45 | 0 | 0.012323564 | 0.259 |
| CmpA_10uM_24h | REACTOME_GLUCOSE_METABOLISM | 44 | 0 | 0.0129953325 | 0.278 |
| CmpA_10uM_24h | REACTOME_FORMATION_OF_TUBULIN_FOLDING_INTERMEDIATES_BY_CCT_TRIC | 16 | 0 | 0.01465286 | 0.318 |
| CmpA_10uM_24h | REACTOME_SIGNALING_BY_WNT | 56 | 0 | 0.017057933 | 0.367 |
| CmpA_10uM_24h | REACTOME_P53_DEPENDENT_G1_DNA_DAMAGE_RESPONSE | 45 | 0.0021834061 | 0.02043924 | 0.439 |
| CmpA_10uM_24h | REACTOME_CYCLIN_E_ASSOCIATED_EVENTS_DURING_G1_S_TRANSITION | 49 | 0 | 0.021482281 | 0.47 |
| CmpA_10uM_24h | REACTOME_SCFSKP2_MEDIATED_DEGRADATION_OF_P27_P21 | 45 | 0 | 0.021505114 | 0.485 |
| CmpA_10uM_24h | REACTOME_PREFOLDIN_MEDIATED_TRANSFER_OF_SUBSTRATE_TO_CCT_TRIC | 22 | 0.0065075923 | 0.025169538 | 0.55 |
| CmpA_10uM_24h | KEGG_GLYCOLYSIS_GLUCONEOGENESIS | 35 | 0.004405286 | 0.031100245 | 0.631 |
| CmpA_10uM_24h | REACTOME_CDT1_ASSOCIATION_WITH_THE_CDC6_ORC_ORIGIN_COMPLEX | 46 | 0 | 0.03237729 | 0.653 |
| CmpA_10uM_24h | REACTOME_ASSEMBLY_OF_THE_PRE_REPLICATIVE_COMPLEX | 52 | 0 | 0.036871538 | 0.713 |
| CmpA_10uM_24h | REACTOME_PURINE_RIBONUCLEOSIDE_MONOPHOSPHATE_BIOSYNTHESIS | 11 | 0 | 0.04194527 | 0.771 |
| CmpA_10uM_24h | REACTOME_REGULATION_OF_MITOTIC_CELL_CYCLE | 60 | 0 | 0.043365564 | 0.811 |
| CmpA_10uM_24h | REACTOME_GLUCOSE_TRANSPORT | 26 | 0.004464286 | 0.044525515 | 0.81 |
| CmpA_10uM_24h | KEGG_PYRIMIDINE_METABOLISM | 54 | 0 | 0.045531593 | 0.809 |
| CmpA_10uM_24h | ST_WNT_CA2_CYCLIC_GMP_PATHWAY | 9 | 0.009940358 | 0.048092753 | 0.865 |
| CmpA_10uM_24h | KEGG_STARCH_AND_SUCROSE_METABOLISM | 17 | 0.002173913 | 0.04869302 | 0.863 |
| CmpA_10uM_24h | REACTOME_HDL_MEDIATED_LIPID_TRANSPORT | 6 | 0 | 0.049485046 | 0.853 |
| CmpA_10uM_24h | KEGG_PENTOSE_PHOSPHATE_PATHWAY | 19 | 0.0060728746 | 0.049632654 | 0.863 |
| CmpA_10uM_24h | REACTOME_TRNA_AMINOACYLATION | 40 | 0.002057613 | 0.04966738 | 0.894 |

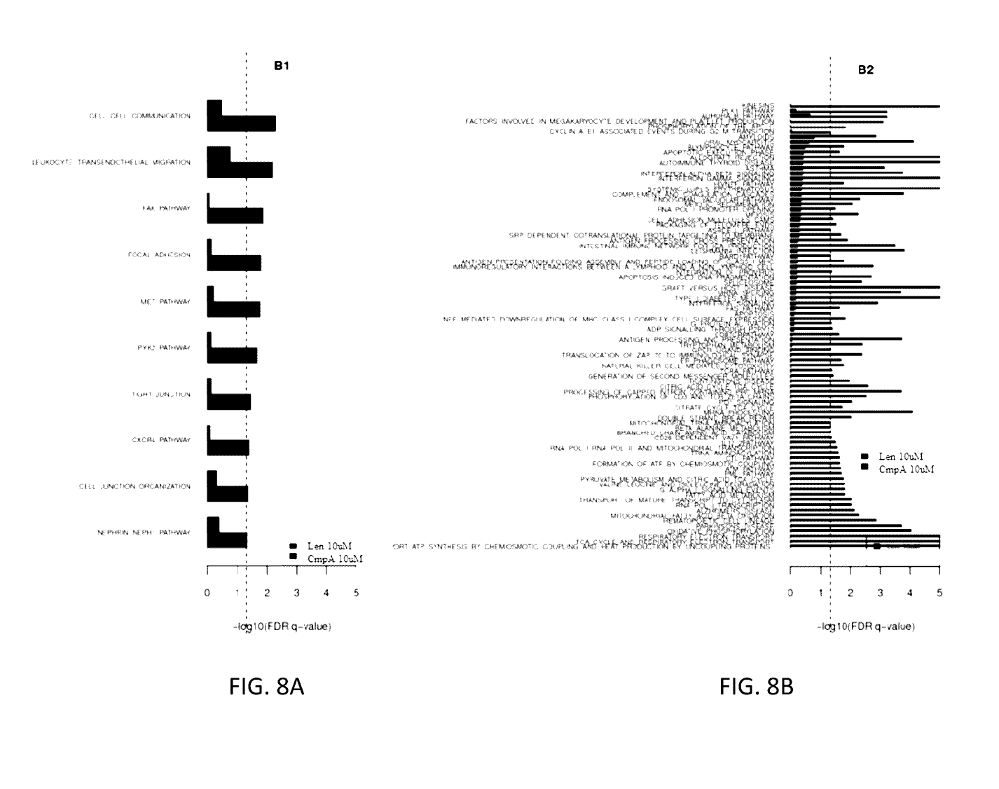

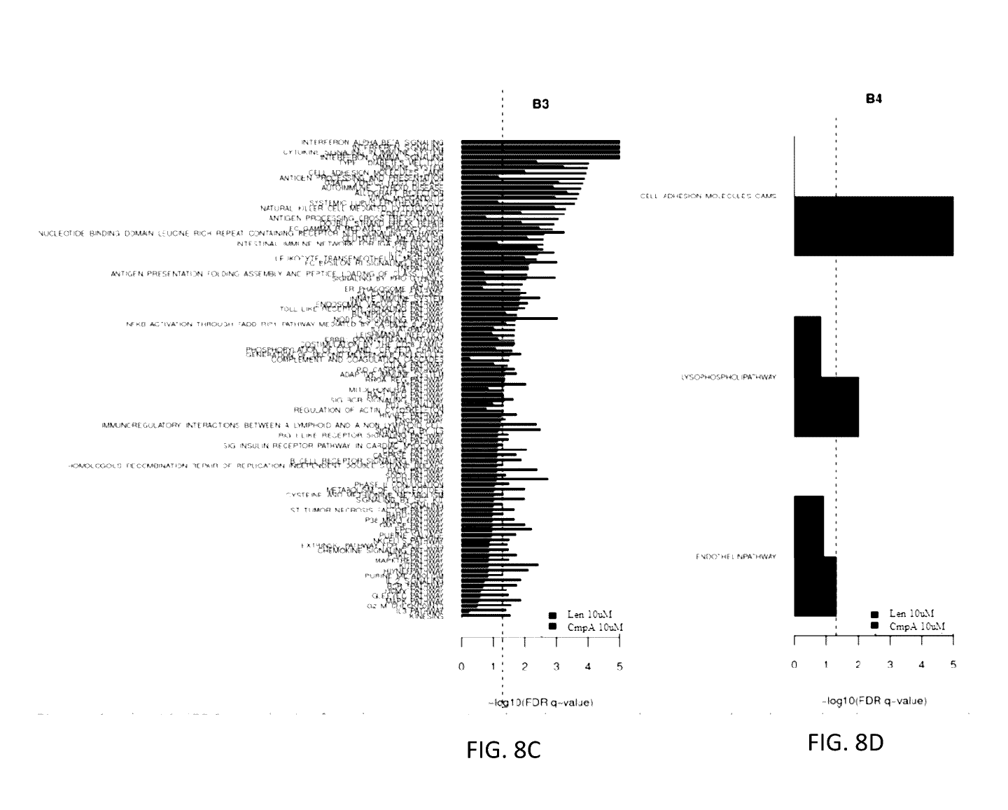

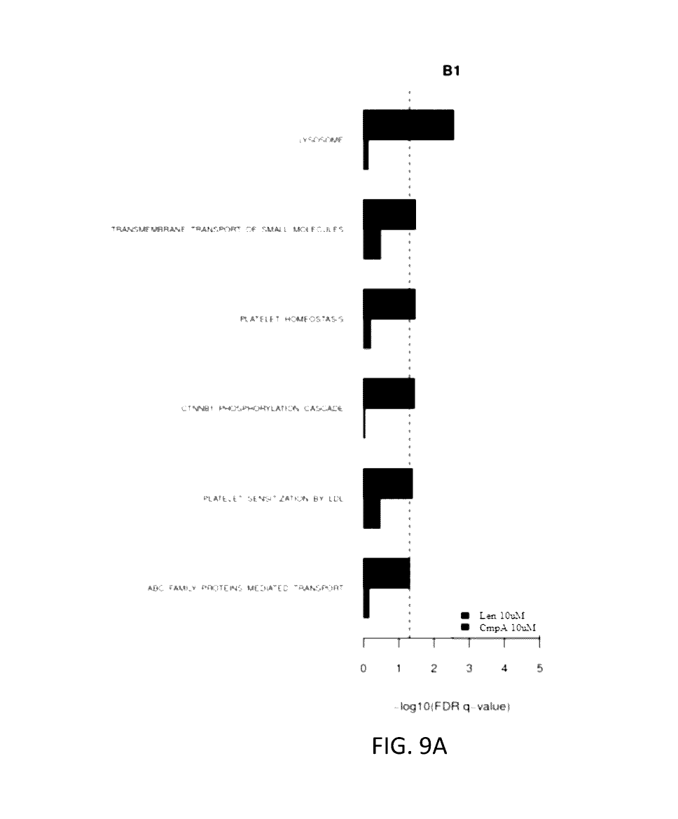

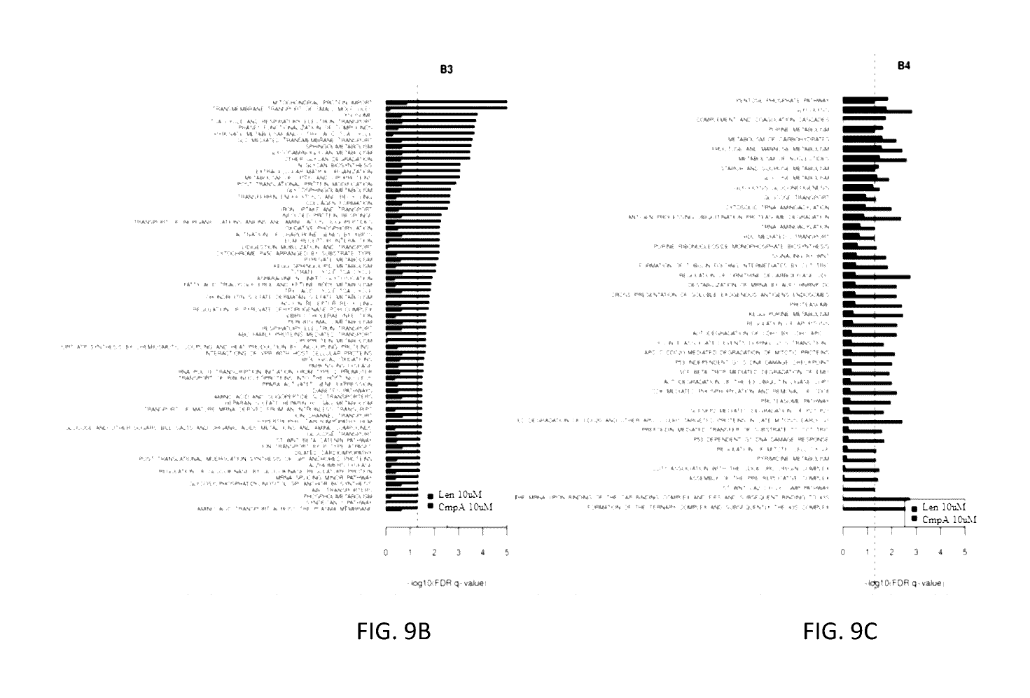

FIGS. 8A-8D and FIGS. 9A-9C show pathways significantly associated with increased and decreased protein abundance upon treatment with 10 μM compound exposure at 24 hours represented as bar-plots for each case scenario. FIGS. 8A-8D show −log 10 (FDR q-value) of pathways associated with increased protein abundance levels upon treatment with lenalidomide or Compound A (10 μM, 24 hours). Vertical dashed line represents the significance cut-off (FDR 5%). FIGS. 9A-9C show −log 10 (FDR q-value) of pathways significantly associated with decreased protein abundance upon treatment with lenalidomide or Compound A (10 μM 24 hours). Vertical dashed line represents the significance cut-off (FDR 5%). Bar-plot for Cases B1 (FIG. 9A), B3 (FIG. 9B), and B4 (FIG. 9C) are shown. No significant pathways are identified in Case B2.

Pathways consistently perturbed by lenalidomide across case scenarios include down-regulation of processes associated to protein transport, lysosomes and endosomal vacuolar pathway and up-regulation of interferon signaling pathway. Interferon signaling pathways are significantly associated with up-regulation in Case B3 reflecting increased levels of interferon related proteins upon compound exposure in the scenario with higher response to Compound A. Kinesins and megakaryocyte development are significantly associated with up-regulation in Case B2, probably linked to IKZF1/IKZF3 degradation and their role in megakaryopoiesis inhibition (see Malinge et al., Blood, 121(13): 2440-2451 (2013). Small overlap between significant processes perturbed upon Compound A and lenalidomide exposure in Case B2 is linked to the low correlation between both compound effects in this particular scenario. Pathway overlap for the two compounds is higher for the other case scenarios, reflecting the alteration of similar general mechanisms upon exposure to both compounds. When comparing case scenarios by pairs there is a small overlap of the processes altered, indicating different responses of different groups of patients to compound treatment. However, Cases B2 and B3 share, among others, pathways related to immune response, interferon signaling, cell regulation and apoptosis, supporting the hypothesis of an stronger leukocyte alteration in these cases as reflected by the strong changes on leukocyte trafficking and inflammation.

The hypothesis of differences in interferon response in sensitive/resistant patients is consistent with functional analyses that associate interferon signaling processes with increased protein abundance levels in patients responsive to Compound A. Experimental validation of interferon-stimulated gene up-regulation by Compound A may guide in the identification of putative early response biomarkers in CLL patients. For example, STAT1 presents an increase in protein abundance upon Compound A exposure for the sensitive patient sample (Case B3) (log 2 fold-change Compound A vs DMSO=0.547; FDR adjusted q-value=0.012) and no changes in protein abundance in the resistant scenario (Case B4) (log 2 fold-change CC-122 vs DMSO=−0.018; FDR adjusted q-value=0.919). Experimental validation of interferon-stimulated gene up-regulation by Compound A may guide in the identification of putative early response biomarkers in CLL patients.

### 6.4 Example 4

### Downregulation of PDE6D in Response to Lenalidomide or Compound D Correlated with Inhibition of Cell Growth by the Treatment Compound

### Methods

Cell lines: Ten different CLL cell lines, including EHEB, I83-E95, Mec2, CII, CI, WA-C3-CD5, WA-OSEL, Mec1, PGA1, and HG3, were categorized into four buckets (Bucket #1, Bucket #2, Bucket #3, and Bucket #4) based on their responsiveness to the treatment of lenalidomide or Compound A. Bucket #1 (EHEB and I83-E95) is responsive to both compounds and exhibits similar EC₅₀ values in response to Compound A compared to lenalidomide. Bucket #2 (Mec2, CII, and CI) is responsive to both compounds and is more sensitive to Compound A than to lenalidomide. Bucket #3 (WA-C3-CD5, WA-OSEL, Mec1, and PGA1) is responsive to Compound A but not to lenalidomide. Bucket #4 (HG3) is not responsive to either Compound A or lenalidomide.

Thymidine incorporation assay: The effects of lenalidomide and Compound A on cell proliferation was assessed by thymidine incorporation assay. Briefly, cells were plated at a density of 1×10⁵ per ml and treated with 1 μM Compound A or 10 μM lenalidomide, four replicates per condition. After four days, cells were diluted 1:7 with fresh media, containing the same concentration of treatment compound. Three days later, ³H-thymidine was added to label proliferating cells for 6-7 hours. Cells were then harvested onto filter plates for counting on a scintillation counter.

Western blot: Cells were treated with 1 μM Compound A or 10 μM lenalidomide for 72 hours. Cells were then harvested and processed for SDS-PAGE and western blot. PDE6D antibody was purchased from Pierce and used at 1:300. For western blot quantification, the band intensity was measured using an Odyssey imager and then normalized to β-actin loading controls.

Scatter plot: The growth of cells treated with 1 μM Compound A or 10 μM lenalidomide as assessed by thymidine incorporation assay was normalized to DMSO controls, and then plotted against the residual PDE6D protein levels normalized to DMSO controls as assessed by western blot.

### Results

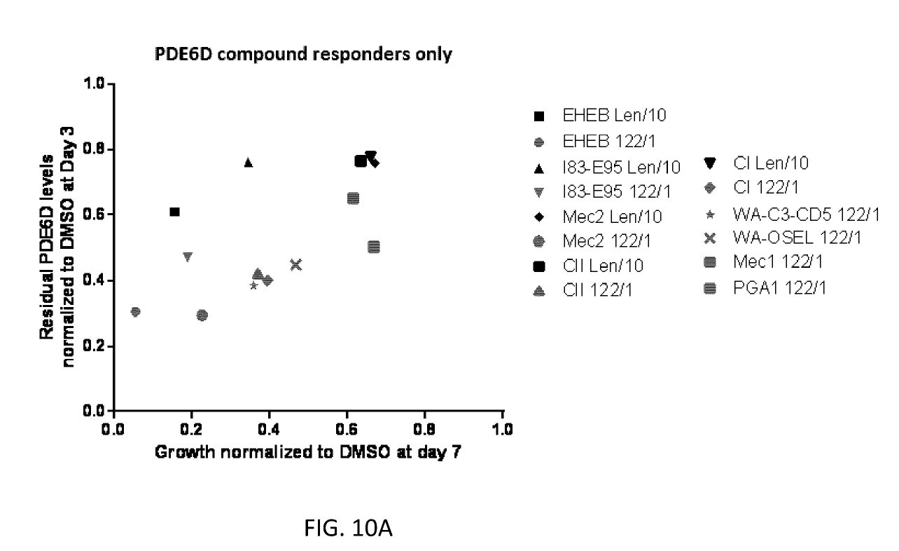

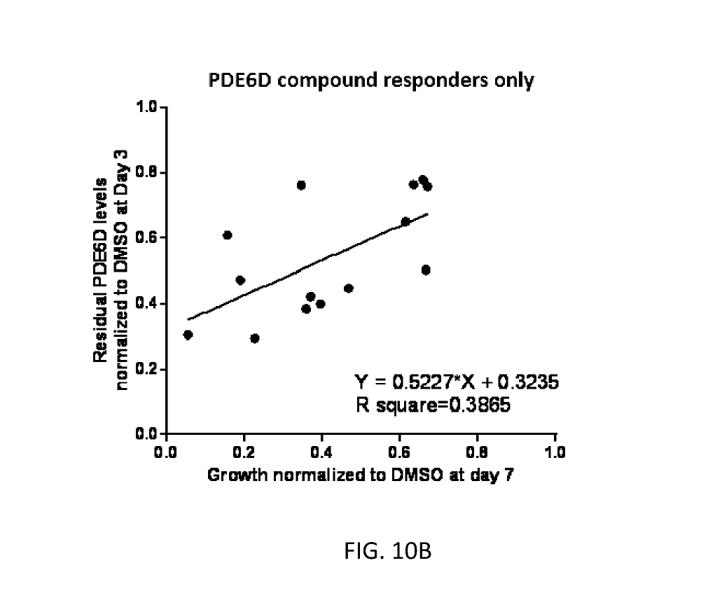

FIG. 10A shows the scatter plot of cells responding to lenalidomide or Compound A. Only data points for cells responding to compound treatment, i.e., cells in Buckets #1 and #2 treated with lenalidomide or Compound A and cells in Bucket #3 treated with Compound A, were plotted. For cells responding to both lenalidomide and Compound A (EHEB, I83-E95, Mec2, CII, and CI), in general, more cell growth inhibition and more PDE6D reduction were observed for treatment with 1 μM Compound A than treatment with 10 μM lenalidomide. The difference between treatment with 1 μM Compound A and treatment with 10 μM lenalidomide was more significant in Bucket #2 (Mec2, CII, and CI) than Bucket #1 (EHEB and I83-E95).

FIG. 10B shows linear regression of the correlation of PDE6D downregulation with growth inhibition in response to treatment of lenalidomide or Compound A. Thus, a reduced level of PDE6D protein can be used as a biomarker to identify a CLL patient who is responsive to a treatment compound, to predict the responsiveness of the patient to the treatment compound, or to direct treatment of the patient by indicating the extent of cell growth inhibition induced by the treatment compound.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

1. A method of identifying a subject having chronic lymphocytic leukemia (CLL) who is likely to be responsive to a treatment compound, predicting the responsiveness of a subject having or suspected of having CLL to a treatment compound, or treating CLL in a subject, comprising:
(a) obtaining a first sample and a second sample from the subject having or suspected of having CLL;
(b) administering 3-(5-amino-2-methyl-4-oxo-4H-quinazolin-3-yl)-piperidine-2,6-dione (Compound A) to the first sample and administering lenalidomide to the second sample;
(c) determining the level of a biomarker in the first sample and determining the level of the biomarker in the second sample, wherein the biomarker is selected from the group consisting of BCL2L1, GZMB, IGHM, IKZF1, IRF8, KLF13, LGALS9, MARCKS, NDE1, NFKBIE, PTK2B, SAMSN1, SELL, SLAMF1, SNX20, SOD2, TCF7, TRAF1, WIZ, ZBTB10, IKZF3, CD40, CR2, CTSS, GBP1, ISG20, SASH1, and SEMA7A;
(d) diagnosing the subject as being likely to be responsive to the treatment compound if the level of the biomarker in the first sample is different from the level of the biomarker in the second sample; and
(e) administering a therapeutically effective amount of the treatment compound to the subject diagnosed to be likely to be responsive to the treatment compound;
wherein the treatment compound is Compound A or lenalidomide.

2-3. (canceled)

4. The method of claim 1, wherein the biomarker is selected from the group consisting of BCL2L1, GZMB, IGHM, IKZF1, IRF8, KLF13, LGALS9, MARCKS, NDE1, NFKBIE, PTK2B, SAMSN1, SELL, SLAMF1, SNX20, SOD2, TCF7, TRAF1, WIZ, and ZBTB10, and diagnosing the subject as being likely to be responsive to both Compound A and lenalidomide if the level of the biomarker in the first sample is different from the level of the biomarker in the second sample.

5. (canceled)

6. The method of claim 4, wherein the biomarker is selected from the group consisting of BCL2L1, MARCKS, NDE1, and ZBTB10, and diagnosing the subject as being likely to be responsive to both Compound A and lenalidomide if the level of the biomarker in the first sample is higher than the level of the biomarker in the second sample.

7. The method of claim 4, wherein the biomarker is selected from the group consisting of GZMB, IGHM, IRF8, KLF13, LGALS9, NFKBIE, SNX20, TCF7, and WIZ, and diagnosing the subject as being likely to be responsive to both Compound A and lenalidomide if the level of the biomarker in the first sample is lower than the level of the biomarker in the second sample.

8-20. (canceled)

21. The method of claim 4 comprising administering a therapeutically effective amount of Compound A or lenalidomide to the subject diagnosed to be likely to be responsive to both Compound A and lenalidomide.

22. The method of claim 1, wherein the biomarker is selected from the group consisting of IKZF1 IKZF3, CD40, CR2, CTSS, GBP1, ISG20, PTK2B, SAMSN1, SASH1, SELL, SEMA7A, SLAMF1, SOD2, and TRAF1, and diagnosing the subject as being likely to be more responsive to Compound A than to lenalidomide if the level of the biomarker in the first sample is different from the level of the biomarker in the second sample.

23. (canceled)

24. The method of claim 22, wherein the biomarker is selected from the group consisting of CD40, CR2, CTSS, GBP1, ISG20, SASH1, and SEMA7A, and diagnosing the subject as being likely to be more responsive to Compound A than to lenalidomide if the level of the biomarker in the first sample is higher than the level of the biomarker in the second sample.

25. The method of claim 22, wherein the biomarker is IKZF3, and diagnosing the subject as being likely to be more responsive to Compound A than to lenalidomide if the level of the biomarker in the first sample is lower than the level of the biomarker in the second sample.

26-32. (canceled)

33. The method of claim 22 comprising administering a therapeutically effective amount of Compound A to the subject diagnosed to be likely to be more responsive to Compound A than to lenalidomide.

34-35. (canceled)

36. The method of claim 1, wherein the biomarker is selected from the group consisting of PTK2B, SAMSN1, SLAMF1, SOD2, and TRAF1, and diagnosing the subject as being likely to be responsive to Compound A if the level of the biomarker in the first sample is higher than the level of the biomarker in the second sample.

37. The method of claim 1, wherein the biomarker is selected from the group consisting of IKZF1 and SELL, and diagnosing the subject as being likely to be responsive to Compound A if the level of the biomarker in the first sample is lower than the level of the biomarker in the second sample.

38. The method of claim 37, wherein the biomarker is IKZF1.

39-45. (canceled)

46. The method of claim 1, wherein the level of the biomarker is determined by comparing to a reference level of the biomarker of a control sample, and wherein the control sample is obtained from the subject prior to administering Compound A or lenalidomide; and wherein the control sample is from the same source as the first and the second samples.

47. The method of claim 1, wherein the level of the biomarker is determined by comparing to a reference level of the biomarker of a control sample, and wherein the control sample is obtained from a healthy subject not having CLL; and wherein the control sample is from the same source as the first and the second samples.

48. (canceled)

49. A method of identifying a subject having chronic lymphocytic leukemia (CLL) who is likely to be responsive to Compound A, predicting the responsiveness of a subject having or suspected of having CLL to Compound A, or treating CLL in a subject, comprising:
(a) obtaining a sample from the subject having or suspected of having CLL;
(b) administering Compound A to the sample;
(c) determining the level of a biomarker in the sample, wherein the biomarker is selected from the group consisting of APOBEC3G, APOC3, APOL2, CR2, CTSS, FCHSD2, GBP2, GBP4, ICAM1, IDI1, IGHM, IKZF1, IKZF3, IL4I1, IRF5, IRF8, ISG20, KYNU, LAP3, LGALS9, NCF2, NCF4, NDE1, NECAP2, OAS1, PARP14, PDE6D, PLEK, PNP, PPA1, PPP1R18, RELB, SAMSN1, SEMA7A, SLFN5, SOD2, TAPBP, TNIP1, TRAF1, TRIP10, and ZFP91; and
(d) diagnosing the subject as being likely to be responsive to Compound A if the level of the biomarker in the sample is different from a reference level of the biomarker in a control sample, wherein the control sample is obtained from a subject not responsive to Compound A; and
(e) administering a therapeutically effective amount of Compound A to the subject diagnosed to be likely to be responsive to Compound A.

50-52. (canceled)

53. The method of claim 49, wherein the biomarker is GBP4.

54. The method of claim 49, wherein the biomarker is LGALS9.

55. The method of claim 49, wherein the biomarker is IRF5.

56. A method of identifying a subject having CLL who is likely to be responsive to a treatment compound, predicting the responsiveness of a subject having or suspected of having CLL to a treatment compound, or treating CLL in a subject, comprising:
(a) obtaining a sample from the subject having or suspected of having CLL;
(b) administering the treatment compound to the sample;
(c) determining the level of a biomarker in the sample, wherein the biomarker is PDE6D; and
(d) diagnosing the subject as being likely to be responsive to the treatment compound if the level of the biomarker in the sample is different from a reference level of the biomarker in a control sample, wherein the control sample is obtained from a subject not responsive to the treatment compound; and
(e) administering a therapeutically effective amount of the treatment compound to the subject diagnosed to be likely to be responsive to the treatment compound;
wherein the treatment compound is Compound A or lenalidomide.

57-58. (canceled)

59. The method of claim 56, wherein the level of the biomarker in the sample is lower than the level of the biomarker in the control sample.

60-66. (canceled)
